SEQUENCE LISTING (264 sequences)

SEQ ID NO: 1 - protein, 15 aa, Synthetic construct
KASQSVDYDGDSYLN

SEQ ID NO: 2 - protein, 7 aa, Synthetic construct
DASNLVS

SEQ ID NO: 3 - protein, 9 aa, Synthetic construct
QQSTEDPWT

SEQ ID NO: 4 - protein, 5 aa, Synthetic construct
SYWMN

SEQ ID NO: 5 - protein, 17 aa, Synthetic construct
QIWPGDGDTNYNGKFKG

SEQ ID NO: 6 - protein, 15 aa, Synthetic construct
RETTTVGRYYYAMDY

SEQ ID NO: 7 - protein, 111 aa, Synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 8 - protein, 124 aa, Synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 9 - protein, 5 aa, Synthetic construct
RYTMH

SEQ ID NO: 10 - protein, 17 aa, Synthetic construct
YINPSRGYTNYNQKFKD

SEQ ID NO: 11 - protein, 10 aa, Synthetic construct
YYDDHYCLDY

SEQ ID NO: 12 - protein, 10 aa, Synthetic construct
RASSSVSYMN

SEQ ID NO: 13 - protein, 7 aa, Synthetic construct
DTSKVAS

SEQ ID NO: 14 - protein, 9 aa, Synthetic construct
QQWSSNPLT

SEQ ID NO: 15 - protein, 119 aa, Synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 16 - protein, 108 aa, Synthetic construct
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 17 - protein, 504 aa, Synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 18 - protein, 14 aa, Synthetic construct
GSSTGAVTSGNYPN

SEQ ID NO: 19 - protein, 7 aa, Synthetic construct
GTKFLAP

SEQ ID NO: 20 - protein, 9 aa, Synthetic construct
VLWYSNRWV

SEQ ID NO: 21 - protein, 5 aa, Synthetic construct
KYAMN

SEQ ID NO: 22 - protein, 19 aa, Synthetic construct
RIRSKYNNYATYYADSVKD

SEQ ID NO: 23 - protein, 14 aa, Synthetic construct
HGNFGNSYISYWAY

SEQ ID NO: 24 - protein, 125 aa, Synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 25 - protein, 109 aa, Synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 26 - protein, 249 aa, Synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 27 - protein, 122 aa, Synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSS

SEQ ID NO: 28 - protein, 122 aa, Synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 29 - protein, 5 aa, Synthetic construct
NYGMN

SEQ ID NO: 30 - protein, 17 aa, Synthetic construct
WINTYTGEPTYADKFQG

SEQ ID NO: 31 - protein, 13 aa, Synthetic construct
WSWSDGYYVYFDY

SEQ ID NO: 32 - protein, 113 aa, Synthetic construct
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 33 - protein, 113 aa, Synthetic construct
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 34 - protein, 17 aa, Synthetic construct
KSSQSVLDSSTNKNSLA

SEQ ID NO: 35 - protein, 7 aa, Synthetic construct
WASTRES

SEQ ID NO: 36 - protein, 9 aa, Synthetic construct
QQSAHFPIT

SEQ ID NO: 37 - protein, 250 aa, Synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIK

SEQ ID NO: 38 - protein, 250 aa, Synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 39 - protein, 505 aa, Synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 40 - protein, 530 aa, Synthetic construct
MGWSCIILFLVATATGVHSQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 41 - protein, 993 aa, Synthetic construct
QVQLVQSGAEVKKPGESVKVSCKASGYTFTNYGMNWVKQAPGQCLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 5 aa, Synthetic construct
NYGMH

SEQ ID NO: 43 - protein, 17 aa, Synthetic construct
VIWYDGSDKYYADSVRG

SEQ ID NO: 44 - protein, 15 aa, Synthetic construct
DGYDILTGNPRDFDY

SEQ ID NO: 45 - protein, 16 aa, Synthetic construct
RSSQSLVHSDGNTYLS

SEQ ID NO: 46 - protein, 7 aa, Synthetic construct
RISRRFS

SEQ ID NO: 47 - protein, 9 aa, Synthetic construct
MQSTHVPRT

SEQ ID NO: 48 - protein, 124 aa, Synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 49 - protein, 112 aa, Synthetic construct
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 50 - protein, 251 aa, Synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 51 - protein, 506 aa, Synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 52 - protein, 994 aa, Synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSSGGGGSGGGGSGGGGSDTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 5 aa, Synthetic construct
DYYMT

SEQ ID NO: 54 - protein, 17 aa, Synthetic construct
YISSSGSTIYYADSVKG

SEQ ID NO: 55 - protein, 8 aa, Synthetic construct
DRNSHFDY

SEQ ID NO: 56 - protein, 11 aa, Synthetic construct
RASQGINTWLA

SEQ ID NO: 57 - protein, 7 aa, Synthetic construct
GASGLQS

SEQ ID NO: 58 - protein, 9 aa, Synthetic construct
QQAKSFPRT

SEQ ID NO: 59 - protein, 117 aa, Synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSS

SEQ ID NO: 60 - protein, 107 aa, Synthetic construct
DIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 61 - protein, 239 aa, Synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIK

SEQ ID NO: 62 - protein, 494 aa, Synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 63 - protein, 982 aa, Synthetic construct
QVQLVESGGGLVKPGGSLRLSCAASGFTFSDYYMTWIRQAPGKGLEWLSYISSSGSTIYYADSVKGRFTISRDNAKNSLFLQMNSLRAEDTAVYYCARDRNSHFDYWGQGTLVTVSSGGGGSGGGGSGGGGSDIQMTQSPSSVSASVGDRVTITCRASQGINTWLAWYQQKPGKAPKLLIYGASGLQSGVPSRFSGSGSGTDFTLTISSLQPEDFATYYCQQAKSFPRTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 982 aa, Synthetic construct
QVQLVESGGGLVKPGGSLRLSCAASGFTFSDHYMSWIRQAPGKCLEWFSYISSSGGIIYYADSVKGRFTISRDNAKNSLYLQMNSLRAEDTAVYYCARDVGSHFDYWGQGTLVTVSSGGGGSGGGGSGGGGSDIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 5 aa, Synthetic construct
SYGMH

SEQ ID NO: 66 - protein, 17 aa, Synthetic construct
FIWYEGSNKYYAESVKD

SEQ ID NO: 67 - protein, 16 aa, Synthetic construct
RAGIIGTIGYYYGMDV

SEQ ID NO: 68 - protein, 11 aa, Synthetic construct
SGDRLGEKYTS

SEQ ID NO: 69 - protein, 7 aa, Synthetic construct
QDTKRPS

SEQ ID NO: 70 - protein, 9 aa, Synthetic construct
QAWESSTVV

SEQ ID NO: 71 - protein, 125 aa, Synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSS

SEQ ID NO: 72 - protein, 107 aa, Synthetic construct
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 73 - protein, 247 aa, Synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLS

SEQ ID NO: 74 - protein, 507 aa, Synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 75 - protein, 989 aa, Synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 987 aa, Synthetic construct
QVQLVESGGGVVQPGGSLRLSCAASGFTFSSYGMHWVRQAPGKGLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGGGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 125 aa, Synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSS

SEQ ID NO: 78 - protein, 106 aa, Synthetic construct
SYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 79 - protein, 246 aa, Synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVL

SEQ ID NO: 80 - protein, 501 aa, Synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 81 - protein, 989 aa, Synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 987 aa, Synthetic construct
QVQLVESGGGVVQPGGSLRLSCAASGFTFSSYGMHWVRQAPGKCLEWVAFIWYEGSNKYYAESVKDRFTISRDNSKNTLYLQMNSLRAEDTAVYYCARRAGIIGTIGYYYGMDVWGQGTTVTVSSGGGGSGGGGSGGGGSSYELTQPPSVSVSPGQTASITCSGDRLGEKYTSWYQQRPGQSPLLVIYQDTKRPSGIPERFSGSNSGNTATLTISGTQAMDEADYYCQAWESSTVVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 7 aa, Synthetic construct
NARMGVS

SEQ ID NO: 84 - protein, 16 aa, Synthetic construct
HIFSNDEKSYSTSLKN

SEQ ID NO: 85 - protein, 14 aa, Synthetic construct
IVGYGSGWYGFFDY

SEQ ID NO: 86 - protein, 11 aa, Synthetic construct
RASQGIRNDLG

SEQ ID NO: 87 - protein, 7 aa, Synthetic construct
AASTLQS

SEQ ID NO: 88 - protein, 9 aa, Synthetic construct
LQHNSYPLT

SEQ ID NO: 89 - protein, 124 aa, Synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 90 - protein, 107 aa, Synthetic construct
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 91 - protein, 246 aa, Synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 92 - protein, 501 aa, Synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 93 - protein, 989 aa, Synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSSGGGGSGGGGSGGGGSDIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 5 aa, Synthetic construct
SYYWS

SEQ ID NO: 95 - protein, 16 aa, Synthetic construct
YVYYSGTTNYNPSLKS

SEQ ID NO: 96 - protein, 10 aa, Synthetic construct
IAVTGFYFDY

SEQ ID NO: 97 - protein, 12 aa, Synthetic construct
RASQRVNNNYLA

SEQ ID NO: 98 - protein, 7 aa, Synthetic construct
GASSRAT

SEQ ID NO: 99 - protein, 9 aa, Synthetic construct
QQYDRSPLT

SEQ ID NO: 100 - protein, 118 aa, Synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSS

SEQ ID NO: 101 - protein, 108 aa, Synthetic construct
EIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 102 - protein, 241 aa, Synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIK

SEQ ID NO: 103 - protein, 496 aa, Synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 104 - protein, 982 aa, Synthetic construct
QVQLQESGPGLVKPSETLSLTCTVSGGSISSYYWSWIRQPPGKCLEWIGYVYYSGTTNYNPSLKSRVTISVDTSKNQFSLKLSSVTAADTAVYYCASIAVTGFYFDYWGQGTLVTVSSGGGGSGGGGSGGGGSEIVLTQSPGTLSLSPGERVTLSCRASQRVNNNYLAWYQQRPGQAPRLLIYGASSRATGIPDRFSGSGSGTDFTLTISRLEPEDFAVYYCQQYDRSPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 5 aa, Synthetic construct
SYGMH

SEQ ID NO: 106 - protein, 17 aa, Synthetic construct
VISYEGSNKYYAESVKG

SEQ ID NO: 107 - protein, 13 aa, Synthetic construct
DRGTIFGNYGLEV

SEQ ID NO: 108 - protein, 122 aa, Synthetic construct
QVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSS

SEQ ID NO: 109 - protein, 16 aa, Synthetic construct
RSSQSLLHKNAFNYLD

SEQ ID NO: 110 - protein, 7 aa, Synthetic construct
LGSNRAS

SEQ ID NO: 111 - protein, 9 aa, Synthetic construct
MQALQTPFT

SEQ ID NO: 112 - protein, 112 aa, Synthetic construct
DIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIK

SEQ ID NO: 113 - protein, 525 aa, Synthetic construct
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 114 - protein, 1013 aa, Synthetic construct
MDMRVPAQLLGLLLLWLRGARCDIVMTQSPLSLPVISGEPASISCRSSQSLLHKNAFNYLDWYLQKPGQSPQLLIYLGSNRASGVPDRFSGSGSGTDFTLKISRVEAEDVGVYYCMQALQTPFTFGCGTKVDIKGGGGSGGGGSGGGGSQVQLVESGGGVVQPGRSLRLSCAASGFTFSSYGMHWVRQAPGKCLEWVAVISYEGSNKYYAESVKGRFTISRDNSKNTLYLQMNSLRDEDTAVYYCARDRGTIFGNYGLEVWGQGTTVTVSSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 5 aa, Synthetic construct
SYPIN

SEQ ID NO: 116 - protein, 16 aa, Synthetic construct
VIWTGGGTNYASSVKG

SEQ ID NO: 117 - protein, 15 aa, Synthetic construct
SRGVYDFDGRGAMDY

SEQ ID NO: 118 - protein, 17 aa, Synthetic construct
KSSQSLLYSSNQKNYFA

SEQ ID NO: 119 - protein, 7 aa, Synthetic construct
WASTRES

SEQ ID NO: 120 - protein, 9 aa, Synthetic construct
QQYYSYPYT

SEQ ID NO: 121 - protein, 123 aa, Synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSS

SEQ ID NO: 122 - protein, 113 aa, Synthetic construct
DIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 123 - protein, 251 aa, Synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIK

SEQ ID NO: 124 - protein, 506 aa, Synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 125 - protein, 994 aa, Synthetic construct
EVQLLESGGGLVQPGGSLRLSCAASGFSFSSYPINWVRQAPGKGLEWVGVIWTGGGTNYASSVKGRFTISRDNSKNTVYLQMNSLRAEDTAVYYCAKSRGVYDFDGRGAMDYWGQGTLVTVSSGGGGSGGGGSGGGGSDIVMTQSPDSLAVSLGERATINCKSSQSLLYSSNQKNYFAWYQQKPGQPPKLLIYWASTRESGVPDRFSGSGSGTDFTLTISSLQAEDVAVYYCQQYYSYPYTFGQGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 5 aa, Synthetic construct
NHIIH

SEQ ID NO: 127 - protein, 17 aa, Synthetic construct
YINPYPGYHAYNEKFQG

SEQ ID NO: 128 - protein, 12 aa, Synthetic construct
DGYYRDTDVLDY

SEQ ID NO: 129 - protein, 11 aa, Synthetic construct
QASQDISNYLN

SEQ ID NO: 130 - protein, 7 aa, Synthetic construct
YTSRLHT

SEQ ID NO: 131 - protein, 9 aa, Synthetic construct
QQGNTLPWT

SEQ ID NO: 132 - protein, 121 aa, Synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSS

SEQ ID NO: 133 - protein, 107 aa, Synthetic construct
DIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 134 - protein, 243 aa, Synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIK

SEQ ID NO: 135 - protein, 498 aa, Synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 136 - protein, 986 aa, Synthetic construct
QVQLVQSGAEVKKPGASVKVSCKASGYTFTNHIIHWVRQAPGQCLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGCGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 5 aa, Synthetic construct
DYYMY

SEQ ID NO: 138 - protein, 17 aa, Synthetic construct
IISDAGYYTYYSDIIKG

SEQ ID NO: 139 - protein, 12 aa, Synthetic construct
GFPLLRHGAMDY

SEQ ID NO: 140 - protein, 11 aa, Synthetic construct
KASQNVDANVA

SEQ ID NO: 141 - protein, 7 aa, Synthetic construct
SASYVYW

SEQ ID NO: 142 - protein, 9 aa, Synthetic construct
QQYDQQLIT

SEQ ID NO: 143 - protein, 121 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 144 - protein, 107 aa, Synthetic construct
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 145 - protein, 243 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 146 - protein, 498 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 147 - protein, 986 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 984 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 498 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 150 - protein, 986 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 984 aa, Synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 5 aa, Synthetic construct
DYYMY

SEQ ID NO: 153 - protein, 17 aa, Synthetic construct
IISDGGYYTYYSDIIKG

SEQ ID NO: 154 - protein, 12 aa, Synthetic construct
GFPLLRHGAMDY

SEQ ID NO: 155 - protein, 11 aa, Synthetic construct
KASQNVDTNVA

SEQ ID NO: 156 - protein, 7 aa, Synthetic construct
SASYVYW

SEQ ID NO: 157 - protein, 9 aa, Synthetic construct
QQYDQQLIT

SEQ ID NO: 158 - protein, 121 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 159 - protein, 107 aa, Synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 160 - protein, 243 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 161 - protein, 498 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 162 - protein, 986 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 984 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 498 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 165 - protein, 986 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 984 aa, Synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 5 aa, Synthetic construct
DYYMY

SEQ ID NO: 168 - protein, 17 aa, Synthetic construct
IISDGGYYTYYSDIIKG

SEQ ID NO: 169 - protein, 12 aa, Synthetic construct
GFPLLRHGAMDY

SEQ ID NO: 170 - protein, 11 aa, Synthetic construct
KASQNVDTNVA

SEQ ID NO: 171 - protein, 7 aa, Synthetic construct
SASYVYW

SEQ ID NO: 172 - protein, 9 aa, Synthetic construct
QQYDQQLIT

SEQ ID NO: 173 - protein, 121 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 174 - protein, 107 aa, Synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 175 - protein, 243 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 176 - protein, 498 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 177 - protein, 986 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 984 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 498 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVL

SEQ ID NO: 180 - protein, 986 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 984 aa, Synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYCGQGTLVTVSSGGGGSGGGGSGGGGSQTVVTQEPSLTVSPGGTVTLTCGSSTGAVTSGNYPNWVQQKPGQCPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 10 aa, Synthetic construct
DKTHTCPPCP

SEQ ID NO: 183 - protein, 12 aa, Synthetic construct
ERKCCVECPPCP

SEQ ID NO: 184 - protein, 16 aa, Synthetic construct
ELKTPLDTTHTCPRCP

SEQ ID NO: 185 - protein, 17 aa, Synthetic construct
ELKTPLGDTTHTCPRCP

SEQ ID NO: 186 - protein, 12 aa, Synthetic construct
ESKYGPPCPSCP

SEQ ID NO: 187 - protein, 5 aa, Synthetic construct
GGGGS

SEQ ID NO: 188 - protein, 10 aa, Synthetic construct
GGGGSGGGGS

SEQ ID NO: 189 - protein, 15 aa, Synthetic construct
GGGGSGGGGSGGGGS

SEQ ID NO: 190 - protein, 20 aa, Synthetic construct
GGGGSGGGGSGGGGSGGGGS

SEQ ID NO: 191 - protein, 25 aa, Synthetic construct
GGGGSGGGGSGGGGSGGGGSGGGGS

SEQ ID NO: 192 - protein, 30 aa, Synthetic construct
GGGGSGGGGSGGGGSGGGGSGGGGSGGGGS

SEQ ID NO: 193 - protein, 35 aa, Synthetic construct
GGGGSGGGGSGGGGSGGGGSGGGGSGGGGSGGGGS

SEQ ID NO: 194 - protein, 40 aa, Synthetic construct
GGGGSGGGGSGGGGSGGGGSGGGGSGGGGSGGGGSGGGGS

SEQ ID NO: 195 - protein, 6 aa, Synthetic construct
PGGGGS

SEQ ID NO: 196 - protein, 6 aa, Synthetic construct
PGGDGS

SEQ ID NO: 197 - protein, 6 aa, Synthetic construct
SGGGGS

SEQ ID NO: 198 - protein, 4 aa, Synthetic construct
GGGG

SEQ ID NO: 199 - protein, 6 aa, Synthetic construct
HHHHHH

SEQ ID NO: 200 - protein, 109 aa, Synthetic construct; identical to SEQ ID NO: 25

SEQ ID NO: 201 - protein, 125 aa, Synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSS

SEQ ID NO: 202 - protein, 249 aa, Synthetic construct
EVQLVESGGGLVQPGGSLRLSCAASGFTFNSYAMNWVRQAPGKGLEWVARIRSKYNNYATYYADSVKGRFTISRDDSKNTAYLQMNSLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 203 - protein, 5 aa, Synthetic construct
GYYWS

SEQ ID NO: 204 - protein, 16 aa, Synthetic construct
DIDASGSTKYNPSLKS

SEQ ID NO: 205 - protein, 12 aa, Synthetic construct
KKYSTVWSYFDN

SEQ ID NO: 206 - protein, 11 aa, Synthetic construct
SGDKLGDKYAS

SEQ ID NO: 207 - protein, 7 aa, Synthetic construct
QDRKRPS

SEQ ID NO: 208 - protein, 9 aa, Synthetic construct
QAWGSSTAV

SEQ ID NO: 209 - protein, 120 aa, Synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSS

SEQ ID NO: 210 - protein, 106 aa, Synthetic construct
SYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 211 - protein, 241 aa, Synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVL

SEQ ID NO: 212 - protein, 496 aa, Synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 213 - protein, 984 aa, Synthetic construct
QVQLQQWGAGLLKPSETLSLTCAVYGGSFSGYYWSWIRQPPGKCLEWIGDIDASGSTKYNPSLKSRVTISLDTSKNQFSLKLNSVTAADTAVYFCARKKYSTVWSYFDNWGQGTLVTVSSGGGGSGGGGSGGGGSSYELTQPSSVSVPPGQTASITCSGDKLGDKYASWYQQKPGQSPVLVIYQDRKRPSGVPERFSGSNSGNTATLTISGTQAMDEADYYCQAWGSSTAVFGCGTKLTVLSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 5 aa, Synthetic construct
GYYMH

SEQ ID NO: 215 - protein, 17 aa, Synthetic construct
WINPNSGGTKYAQKFQG

SEQ ID NO: 216 - protein, 16 aa, Synthetic construct
DRITVAGTYYYYGMDV

SEQ ID NO: 217 - protein, 11 aa, Synthetic construct
RASQGVNNWLA

SEQ ID NO: 218 - protein, 7 aa, Synthetic construct
TASSLQS

SEQ ID NO: 219 - protein, 9 aa, Synthetic construct
QQANSFPIT

SEQ ID NO: 220 - protein, 125 aa, Synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 221 - protein, 107 aa, Synthetic construct
DIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 222 - protein, 247 aa, Synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 223 - skipped

SEQ ID NO: 224 - protein, 502 aa, Synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 225 - protein, 508 aa, Synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 226 - protein, 990 aa, Synthetic construct
QVQLVQSGAEVKKPGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 5 aa, Synthetic construct
GYYMH

SEQ ID NO: 228 - protein, 17 aa, Synthetic construct
WINPNSGGTKYAQKFQG

SEQ ID NO: 229 - protein, 16 aa, Synthetic construct
DRITVAGTYYYYGMDV

SEQ ID NO: 230 - protein, 11 aa, Synthetic construct
RASQGVNNWLA

SEQ ID NO: 231 - protein, 7 aa, Synthetic construct
TASSLQS

SEQ ID NO: 232 - protein, 9 aa, Synthetic construct
QQANSFPIT

SEQ ID NO: 233 - protein, 125 aa, Synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSS

SEQ ID NO: 234 - protein, 107 aa, Synthetic construct; identical to SEQ ID NO: 221

SEQ ID NO: 235 - protein, 247 aa, Synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIK

SEQ ID NO: 236 - protein, 502 aa, Synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 237 - protein, 508 aa, Synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 238 - protein, 990 aa, Synthetic construct
QVQMVQSGAEVKKHGASVKVSCKASGYTFTGYYMHWVRQAPGQCLEWMGWINPNSGGTKYAQKFQGRVTMTRDTSISTAYMELSRLRSDDTAVYYCARDRITVAGTYYYYGMDVWGQGTTVTVSSGGGGSGGGGSGGGGSDIQMTQSPSSVSASVGDRVTITCRASQGVNNWLAWYQQKPGKAPKLLIYTASSLQSGVPSRFSGSGSGTDFTLTIRSLQPEDFATYYCQQANSFPITFGCGTRLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 5 aa, Synthetic construct
TYAMS

SEQ ID NO: 240 - protein, 17 aa, Synthetic construct
AISGSGGRTFYAESVEG

SEQ ID NO: 241 - protein, 11 aa, Synthetic construct
HDYSNYPYFDY

SEQ ID NO: 242 - protein, 12 aa, Synthetic construct
RASQSVRSTYLA

SEQ ID NO: 243 - protein, 7 aa, Synthetic construct
GASSRAT

SEQ ID NO: 244 - protein, 9 aa, Synthetic construct
QQYGDLPFT

SEQ ID NO: 245 - protein, 120 aa, Synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSS

SEQ ID NO: 246 - protein, 108 aa, Synthetic construct
EIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIK

SEQ ID NO: 247 - protein, 242 aa, Synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEI

SEQ ID NO: 248 - protein, 497 aa, Synthetic construct
EVQLLESGGGMVQPGGSLRLSCAASGFTFSTYAMSWVRQAPGKCLEWVSAISGSGGRTFYAESVEGRFTISRDNSKNTLYLQMNSLRAEDTAVYYCAKHDYSNYPYFDYWGQGTLVTVSSGGGGSGGGGSGGGGSEIVLTQSPGTLSLSPGERATLSCRASQSVRSTYLAWYQQKPGQAPRLLIYGASSRATGIPDRFSGSGSGTDFTLTISRLEPEDFAVYSCQQYGDLPFTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTV

SEQ ID NO: 249 - protein, 227 aa, Synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 225 aa, Synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 251 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 225 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 253 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 225 aa, Synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 255 - protein, 227 aa, Synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 225 aa, Synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 257 - protein, 484 aa, Synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 480 aa, Synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 259 - protein, 484 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 480 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 261 - protein, 484 aa, Synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 480 aa, Synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 263 - protein, 484 aa, Synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 480 aa, Synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

(19) United States
(12) Patent Application Publication
(10) Pub. No.: US 2026/0232593 A1
(43) Pub. Date: Aug. 13, 2026

(54) Controlled-Ice Nucleation Lyophilization Process for Bispecific Molecules

(71) Applicant: AMGEN INC., Thousand Oaks, CA (US)

(72) Inventors: Farzad Mohajerani, Thousand Oaks, CA (US); Jason Ricci, Thousand Oaks, CA (US); Debra Wu, Thousand Oaks, CA (US); Ronak Maheshawari, Thousand Oaks, CA (US); Wisam Al-Bakri, Thousand Oaks, CA (US); Arnab Ganguly, Thousand Oaks, CA (US); Philippe Lam, Thousand Oaks, CA (US)

(21) Appl. No.: 19/156,116

(22) PCT Filed: Mar. 8, 2024
(86) PCT No.: PCT/US2024/018992

§ 371 Date: Aug. 13, 2025

Related U.S. Application Data
(60) Provisional application No. 63/488,997, filed on Mar. 8, 2023.

(51) Int. Cl. A61K 9/19; C07K 16/28

(52) U.S. Cl. CPC A61K 9/19; C07K 16/28; C07K 16/2809; C07K 16/2878; C07K 2317/31

(57) ABSTRACT

A process for the production of a chimeric antibody, comprising: a) preparing a replicable expression vector including a suitable promoter operably linked to a DNA sequence comprising a first part which encodes at least the variable region of the heavy or light chain of an Ig molecule and a second part which encodes at least part of a second protein; b) if necessary, preparing a replicable expression vector including a suitable promoter operably linked to a DNA sequence which encodes at least the variable region of a complementary light or heavy chain respectively of an Ig molecule; c) transforming an immortalised mammalian cell line with the or both prepared vectors; and d) culturing said transformed cell line to produce the chimeric antibody; chimeric antibodies produced by this process; and plasmids and transformed cell lines used in the process.

### FIELD

The disclosure relates to a method of lyophilizing a composition comprising bispecific molecules and the resulting lyophilized composition.

### BACKGROUND

Bispecific T cell engaging molecules such as BiTE® molecules have demonstrated clinical benefits in immunooncology. Typically, one binding domain of these molecules is specific for a selected tumor-associated surface antigen on target cells and the second binding domain is specific for CD3, a subunit of the T cell receptor complex on T cells. By their particular design, BiTE® molecules are uniquely suited to transiently connect T cells with target cells and, at the same time, potently activate the inherent cytolytic potential of T cells against target cells. Before storage, bispecific T cell engaging molecules are typically lyophilized. Lyophilization is a manufacturing process widely used to increase the stability of pharmaceutical products by removing the water content from a liquid product via freeze-drying. Lyophilization is a batch process and is typically lengthy, contributing to more than 50% of drug product processing time. In addition, manufacturing scale lyophilizers are limited with respect to capacity; only a certain quantity of vials can be loaded in a batch, and there are limits to the amount of liquid composition that can be dispensed into the vials. Due to these constraints, lyophilized products are typically costly. See, e.g., Awotwe-Otoo et al., International Journal of Pharmaceutics, 450 (2013), 70-78; Esfandiary et al., J. Pharm. Sci., 105 (2016), 1427-1433. Efforts to increase the efficiency of lyophilization processes for biopharmaceutical products such as bispecific T cell engaging molecules may be hindered by inconsistent product attributes, for example, formation of excessive high molecular weight species (HMWS) especially at higher product concentration, which results in expensive loss of product.

The freezing step in a conventional lyophilization process involves uncontrolled or stochastic ice nucleation that occurs at a significantly lower temperature than that of the normal freezing point. Therefore, such “supercooling” results in small ice crystals, requiring long drying times. One approach to reduce overall lyophilization process time is to add an annealing step to the lyophilzation process, which increases the average size of the ice crystals through a process referred to as Ostwald ripenening. However, as the present inventors have found out, such an annealing step may lead to aggregation, i.e. the formation of high molecular weight species (HMWS), typically in high concentration bispecific molecule formulations, e.g. at concentrations of at least 5 mg/ml or even at least 10 mg/ml, likely due to low temperature degradation mechanisms. Hence, there is a need for an improved lyophilization process which provides bispecific T cell engaging molecule product with low rates of aggregated product even if starting from higher concentration formulations.

### SUMMARY

In view of the unmet need described above, it is an object of the present invention to provide a resources-saving method to provide bispecific molecules, preferably bispecific (T cell engaging) molecules comprising a half-life extending Fc domain, at concentrations above 10 mg/ml which feature only low levels (e.g. below or equal 1.5% of total molecule) of undesired aggregates such as HMWS. The problem is solved by introducing a controlled ice nucleation (CIN) step into a lyophilisation process to lyophilize high concentrated bispecific molecule with a Fc domain, to reduce overall process time in comparison to standard lyophilisation processes. Said reduction of process time reduces energy consumption and frees the apparatus sooner for the next run. In a first aspect, it is envisaged in the context of the present invention to provide a method of preparing a lyophilized bispecific molecule composition, the method comprising
(a) a (CIN) step of inducing ice nucleation in a liquid bispecific molecule composition having a concentration of bispecific molecule of at least 10 mg/ml in a vial exposed to a first temperature of about −10° C. to about −18° C. for about 60 minutes to about 270 minutes, wherein this step preferably comprises a post-nucleation hold of up to 90 minutes, preferably about 30 minutes,
(b) a freezing step of exposing the vial to a second temperature of about −25° C. to −50° C. for about 1 to 5 hours;
(c) a drying step of drying the composition of (b) at a third temperature of about −5° C. to about −25° C. for about 25 to 70 hours; and
(d) further drying the composition of (c) at a fourth temperature of about 25° C. to 50° C. for about 4 to 12 hours, resulting in a vial comprising a lyophilized bispecific molecule composition having a percental content of high molecular weight species (HMWS) of less or equal 1.5% (m/V), wherein the bispecific molecule comprises at least three domains, wherein:
a first domain binds to a tumor antigen on a target cell;
a second domain binds to an extracellular epitope of the human and/or the Macaca CD3ε chain; and
a third domain, which preferably provides an extended half-life of the bispecific molecule, fused to the second domain by a peptide linker, which third domain comprises two polypeptide monomers, each comprising a hinge, a CH2 and a CH3 domain, wherein said two polypeptide monomers are fused to each other via a peptide linker.

Within said aspect, it is also envisaged in the context of the present invention to provide a method, wherein step (a) comprises exposing the vial to the first temperature for about 90 minutes to about three hours (CIN step).

Within said aspect, it is also envisaged in the context of the present invention to provide a method, wherein step (a) comprises exposing the vial to the first temperature for about 90 minutes to about two hours, preferably 110 minutes.

Within said aspect, it is also envisaged in the context of the present invention to provide a method, wherein the first temperature in step (a) is about −12° C. to about −17° C.

Within said aspect, it is also envisaged in the context of the present invention to provide a method, wherein the first temperature in step (a) is about −15° C.

Within said aspect, it is also envisaged in the context of the present invention to provide a method, wherein step (a) further comprises holding the ice-nucleated bispecific molecule composition at the temperature for a post-nucleation period of time of up to 90 minutes.

Within said aspect, it is also envisaged in the context of the present invention to provide a method, wherein the post-nucleation period of time is about 20 minutes to about 90 minutes.

Within said aspect, it is also envisaged in the context of the present invention to provide a method, wherein the post-nucleation period of time is about 30 minutes.

Within said aspect, it is also envisaged in the context of the present invention to provide a method, where step (b) comprises exposing the vial to a second temperature of about −45° C. (freeze step).

Within said aspect, it is also envisaged in the context of the present invention to provide a method, wherein the second period of time is about two hours to about four hours.

Within said aspect, it is also envisaged in the context of the present invention to provide a method of claim 10, wherein the second period of time is about three hours.

Within said aspect, it is also envisaged in the context of the present invention to provide a method, wherein the first temperature is transitioned to the second temperature at a rate of about 0.01° C. to about 0.5° C. per minute.

Within said aspect, it is also envisaged in the context of the present invention to provide a method, wherein the first temperature is transitioned to the second temperature at a rate of about 0.2° C. per minute.

Within said aspect, it is also envisaged in the context of the present invention to provide a method, wherein third temperature of step (c) is about 0° C. to about −20° C. (drying step), preferably at about 70 to 120 mTorr, preferably 100 mTorr.

Within said aspect, it is also envisaged in the context of the present invention to provide a method, wherein third temperature of step (c) is about −5° C. to about −10° C.

Within said aspect, it is also envisaged in the context of the present invention to provide a method, wherein third temperature of step (c) is about −8° C.

Within said aspect, it is also envisaged in the context of the present invention to provide a method, wherein step (c) comprises exposing the vial comprising the bispecific molecule composition to an increase in temperature at rate of about 0.01° C. to about 0.5° C. per minute.

Within said aspect, it is also envisaged in the context of the present invention to provide a method, wherein the transition of step (b) to step (c) comprises increasing the temperature at rate of about 0.2° C. to about 0.7° C. per minute and holding the vial at a temperature of about −40° C. to about −30° C. for about 15 minutes to about an hour.

Within said aspect, it is also envisaged in the context of the present invention to provide a method, wherein the liquid bispecific molecule composition is exposed in step (a) to a first temperature of about −15° C. for about 110 minutes plus about 30 minutes post nucleation, is exposed in step (b) to a second temperature of about −45° C. for about 3 hours; the composition of (b) is dried in step (c) at a third temperature of about −8° C. for about 50 hours; and the composition of (c) is further dried at a fourth temperature of about 40° C. for about 8 hours,), wherein step (d) is preferably carried out at about 70 to 120 mTorr, preferably 100 mTorr.

Within said aspect, it is also envisaged in the context of the present invention to provide a method, wherein the bispecific molecule is a single chain molecule.

Within said aspect, it is also envisaged in the context of the present invention to provide a method, wherein the bispecific molecule is present in the composition at a concentration of about 10 mg/ml-30 mg/ml, or about 20 mg/ml to about 30 mg/ml or, preferably about 15 mg/ml to 25 mg/ml.

Within said aspect, it is also envisaged in the context of the present invention to provide a method, wherein the method results in a vial comprising a lyophilized bispecific molecule composition showing aggregation in terms of high molecular weight species (HMSW) formation lower than or equal to about 1.5% (m/w) or preferably lower than or equal of about 1.2, 1, 0.75 or even 0.5% (relative to total bispecific molecule weight).

Within said aspect, it is also envisaged in the context of the present invention to provide a method, wherein said third domain comprises in an amino to carboxyl order: hinge-CH2-CH3-linker-hinge-CH2-CH3.

Within said aspect, it is also envisaged in the context of the present invention to provide a method, wherein each of said polypeptide monomers of the third domain has an amino acid sequence that is at least 90% identical to a sequence selected from the group from the group consisting of: SEQ ID NO: 249-256 or identical to a sequence selected from the group from the group consisting of: SEQ ID NO: 249-256.

Within said aspect, it is also envisaged in the context of the present invention to provide a method, wherein the first domain binds to CD33, CDH19, MSLN, FLT3, BCMA, CD19, MUC17, CDH3, CLDN18.2, CD70, EGFRviii, EpCAM, DLL3 and/or PSMA, preferably DLL3,

Within said aspect, it is also envisaged in the context of the present invention to provide a method, wherein the first binding domain of the construct comprises a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 1, CDR-L2 as depicted in SEQ ID NO: 2 and CDR-L3 as depicted in SEQ ID NO: 3,
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36,
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47,
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58,
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70,
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88,
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99,
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111,
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120,
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131,
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142,
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157,
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172,
(n) CDR-H1 as depicted in SEQ ID NO: 203, CDR-H2 as depicted in SEQ ID NO: 204, CDR-H3 as depicted in SEQ ID NO: 205, CDR-L1 as depicted in SEQ ID NO: 206, CDR-L2 as depicted in SEQ ID NO: 207 and CDR-L3 as depicted in SEQ ID NO: 208;
(o) CDR-H1 as depicted in SEQ ID NO: 214, CDR-H2 as depicted in SEQ ID NO: 215, CDR-H3 as depicted in SEQ ID NO: 216, CDR-L1 as depicted in SEQ ID NO: 217, CDR-L2 as depicted in SEQ ID NO: 218 and CDR-L3 as depicted in SEQ ID NO: 219;
(p) CDR-H1 as depicted in SEQ ID NO: 226, CDR-H2 as depicted in SEQ ID NO: 227, CDR-H3 as depicted in SEQ ID NO: 228, CDR-L1 as depicted in SEQ ID NO: 229, CDR-L2 as depicted in SEQ ID NO: 230 and CDR-L3 as depicted in SEQ ID NO: 231; and
(q) CDR-H1 as depicted in SEQ ID NO: 238, CDR-H2 as depicted in SEQ ID NO: 239, CDR-H3 as depicted in SEQ ID NO: 240, CDR-L1 as depicted in SEQ ID NO: 241, CDR-L2 as depicted in SEQ ID NO: 242 and CDR-L3 as depicted in SEQ ID NO: 243.

Within said aspect, it is also envisaged in the context of the present invention to provide a method, wherein the first domain has an amino acid sequence selected from the group consisting of SEQ ID Nos: 7, 8, 17, 27, 28, 37, 38, 39, 40, 41, 48, 49, 50, 51, 52, 59, 60, 61, 62, 63, 64, 71, 72, 73, 74, 75. 76, 77, 78, 79, 80, 81, 89, 90, 91, 92, 93, 100, 101, 102, 103, 104, 113, 114, 121, 122,123, 124, 125, 131, 132, 133, 134, 135, 136, 143, 144, 145, 146, 147, 148, 149, 150, 151, 158, 159, 160, 161,162, 163, 164, 165, 166, 173, 174, 175, 176, 177, 178, 179,180, 181, 223, 235 and 246, preferably 100 to 104.

Within said aspect, it is also envisaged in the context of the present invention to provide a method, wherein ice nucleation is induced via ice-fog or depressurization.

In a further aspect of the present invention, a lyophilized bispecific molecule composition is prepared via the method described herein.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together, unless context unambiguously requires otherwise.

It should also be understood that when describing a range of values, the disclosure contemplates individual values found within the range. For example, “a pH from about pH 4 to about pH 6,” could be, but is not limited to, pH 4.2, 4.6, 5.2, 5.5, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional embodiments that also are intended as aspects of the invention, irrespective of whether the combination of features is specified as an aspect or embodiment of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention.

### BRIEF DESCRIPTION OF THE DRAWINGS

FIG. 1A-B: (A) A standard lyophilization cycle for bispecific molecules without a CIN step contains annealing during the freezing phase and a longer primary drying time (B) With no annealing step, CIN lyophilization cycles rely on triggering ice nucleation at warmer temperatures to form larger ice crystals, which results in short primary drying and total cycle times.

FIG. 2A-C: Lyophilization profiles of lyophilization cycles for both BCMA×CD3 and DLL3×CD3 bispecific molecule samples. The same lyophilization tray arrangement was used for each lyophilization cycle. (A) Standard non-CIN cycle with annealing, wherein maximum product temperature (Tp) of BCMA×CD3 bispecific molecule was −33.3° C. and Tp max of DLL3×CD3 bispecific molecule was −32.8° C. (B) Standard non-CIN cycle without annealing, wherein maximum product temperature (Tp) of BCMA×CD3 bispecific molecule was −31.3° C. and Tp max of DLL3×CD3 bispecific molecule was −31.5° C. (C) CIN cycle, wherein maximum product temperature (Tp) of BCMA×CD3 bispecific molecule was −27.8° C. and Tp max of DLL3×CD3 bispecific molecule was −28.7° C.

FIG. 3A-B: Change in % HMWS after lyophilization for the standard non-CIN cycle, the standard non-CIN cycle without annealing, and two CIN cycles 1 and 2 with different nucleation conditions and post-nucleation holds. Due to this, the CIN cycle 2 nucleation parameters of −15° C. and 0.5 h post-nucleation were chosen to proceed with experiments. (A) BCMA×CD3 bispecific molecule (B) DLL3×CD3 bispecific molecule.

FIG. 4A-B: Stability results of % HMWS over time for the standard non-CIN cycle, the standard non-CIN cycle without annealing, and the CIN cycle for BCMA×CD3 bispecific molecule and DLL3×CD3 bispecific molecule. Values are % HMWS immediately post-recon recorded at each timepoint. Lines are overlapping in graph. (A) BCMA×CD3 bispecific molecule (B) DLL3×CD3 bispecific molecule.

FIG. 5: Time zero moisture content of BCMA×CD3 bispecific molecule and DLL3×CD3 bispecific molecule samples after lyophilization. No significant difference in moisture content was present between groups (p>0.05).

FIG. 6A-F: BCMA×CD3 bispecific molecule and DLL3×CD3 bispecific molecule CEX-HPLC results. (A) BCMA×CD3 bispecific molecule Main peak (B) BCMA×CD3 bispecific molecule Acidic peak (C) BCMA×CD3 bispecific molecule Basic peak (D) DLL3×CD3 bispecific molecule Main peak (E) DLL3×CD3 bispecific molecule Acidic peak (F) DLL3×CD3 bispecific molecule Basic peak

FIG. 7: Average cake resistance at 1 mm depth in a 6R vial with 1.3 ml fill volume. The cake resistance is lower by >50% when using CIN as compared to the standard lyophilisation process and even significantly lowered compared with standard process with additional annealing. “CIN 1” process with CIN step parameters not according to the present invention had nucleation temperature of −7° C. and, in contrast, “CIN2” process according to the present invention had nucleation temperature of −15° C.

### DETAILED DESCRIPTION

The disclosure provides a method for preparing a lyophilized bispecific molecule composition, in particular a composition comprising a bispecific T cell engaging molecule comprising a half-life extending Fc domain, the method comprising a controlled ice nucleation (CIN) step. CIN was surprisingly found as a better alternative to introducing an annealing step to lyophilisation of bispecific molecules according to the present invention in order to achieve both desirable product quality (i.e. significant reduction in HMWS in comparison to conventional lyophilisation with annealing step) and reduced process time compared to a conventional process without annealing step or CIN step. The process time saving comes from a shorter step (c) and (d) as described herein in comparison to a conventional lyophilisation process without annealing. For example, a conventional lyophilisation process may take up to 100 hours for primary drying step (c), while a process according to the present invention has a shorter drying time, preferably only about 50 hours for step (c). Taken the secondary drying step (d) into consideration, overall time saving of a process ois at least 30% or even more such as 40% with respect to a conventional lyophilisation process. Therefore, the consumption of resources such as energy is likewise reduced. An exemplary conventional or—also referred to herein—as “standard” lyophilisation process, i.e. in general without a CIN step, for bispecific molecules is understood herein as having no CIN or annealing step, a freezing step (b) of e.g. about −45° C. for about 2 hours, a primary drying step (c) e.g. at about −25° C. and about 70 mTorr for about 100 hours and a secondary drying step (d) e.g. at about 40° C. and about 70 mTorr for about 8 hours.

Without wanting to be bound by theory, larger ice crystals and hence shorter cycle time is achieved by introducing the CIN step with the process parameters as disclosed herein. CIN introduces externally formed ice crystals into the drug product vials, hence resulting in larger ice crystals post-freezing. In the present invention, it is shown that applying a CIN step in contrast to, e.g., an annealing step operated under selected conditions geared to bispecific molecules, efficiently mitigates aggregation in terms of HMWS, without impacting other product quality attributes such as lyophilization cake appearance and moisture content. Even more, cake resistance was advantageously reduced, e.g. by at least 30%, typically by about 50% by the present method comprising a CIN step as compared to standard non-CIN lyophilisation with or without an annealing step. The lyophilization cycle with annealing typically causes undesirable aggregation, typically at least 3% HMWS, while a lyophilisation process comprising a CIN step as described herein keeps aggregation, i.e. HMSW, level at or below 1.5% which is close to the pre-lyophilization value of 1%, or even lower as demonstrated in the examples.

The CIN step according to the present invention comprises incubation preferably in the range of −18 to −10° C. for about 1 to 5 hours, typically 90 min to 120 min such as 90, 110 or 120 min, to cool the composition (pre-nucleation) so that ice crystals don't melt when introduced in the next step. Ice crystals are introduced to induce ice nucleation by incubation preferably in the range of −18 to −10° C. for additional time to allow ice crystals to grow (post-nucleation). It is an advantage of the present invention to provide a method comprising a CIN step based on “sweet spot” conditions which facilitate both beneficial bispecific molecule product quality (in terms of product uniformity, preferably without HMWS, at higher product concentrations) as explained and demonstrated herein, and makes the process more efficient regarding shorter drying process length. Higher ice nucleation temperatures above −10° C. such as −7° C.—despite the advantage of faster drying time—typically result in bispecific molecule product with inferior product quality in terms of aggregation. In contrast, a lower ice nucleation temperature below −18° C. may typically lead to longer drying times and may forfeit any benefit in terms of process length and resources as well as energy savings. Some process parameters may have less impact on product quality and process economics in terms of drying time than CIN step temperature and duration. For example, secondary drying pressure may not be set over a wider range as it is known in the art that secondary drying pressure has less impact on drying process and, hence, may not serve to particular save energy.

The method preferably comprises inducing ice nucleation in a liquid bispecific molecule composition in a vial exposed to a first temperature in the range of about −18° C. to about −10° C. (e.g. of about −18, −17, −16, −15, −14, −13, −12, −11, or −10° C.) for about 1 to about five hours, preferably 90 to 120 min; exposing the vial to a second temperature of about −25° C. to −50° C. for a second period of time; and drying the composition at a third temperature of about −5° C. to about −25° C. The method results in a vial comprising a lyophilized bispecific molecule composition of advantageous product parameters (low percental HMWS despite higher and commercially beneficial product concentration) and having an aspect ratio of greater than or equal to about 0.75 (e.g., greater than or equal to 0.8, 0.85, 0.9, or 0.95). In various aspects, the method results in a vial comprising a lyophilized bispecific molecule composition having an aspect ratio of greater than or equal to about 1 (e.g., greater than or equal to 1.1, 1.2, 1.3, 1.4, 1.5, 1.6, 1.7, 1.8, 1.9, or 2). Remarkably, the process disclosed herein allows surprisingly high fill volume for a given vial (i.e., aspect ratios) while minimizing the risks of vial breakage and retaining reasonable drying times. “Aspect ratio” is the fill height of a vial (the height of bispecific molecule composition in the vial) divided by the vial internal diameter (aspect ratio=fill height/vial internal diameter). Previous methods required reduced volumes to be introduced into vials (a lower aspect ratio) to prevent vial breakage and product cake collapse during the lyophilization process. The method described herein allows higher aspect ratios during the lyophilization process, which provides a number of potential advantages. For example, for bispecific molecule therapeutics administered in large doses, fewer vials are required for dosing. Alternatively (or in addition), the process allows use of smaller vials compared to other lyophilization methods while maintaining the same fill volume, thereby resulting in more units per batch and, thus, increased output. Use of smaller vials also provides the benefit of reducing storage space manufacturing sites and clinics. These are merely examples of advantages of the instant method, which are achieved while producing product cakes suitable for biopharmaceutical use.

The disclosure describes various conditions for use in a lyophilization process to produce a lyophilized bispecific molecule composition. Generally, vials (e.g., glass vials) suitable for pharmaceutical compositions are filled with liquid bispecific molecule composition and exposed to different temperatures and pressures to achieve a lyophilized product. The vial may be any size or shape suitable for use in lyophilization processes, and can be formed from a variety of materials, such as glass, metal, or plastic (e.g., polycarbonate, polystyrene, polypropylene, or polyolefine). For example, the vial may be glass or glass-like and tubular in shape. Molded glass vials are commercially available in a range of different sizes with dimensions. Indeed, various sizes of vials are commercially available (e.g., size 2R, 4R, 6R, 8R, 10R, 15R, 20R, 25R, 30R, 50R or 100R). The vial may be constructed to include a suitable stopper, such as a commercially available elastomeric stopper available from, e.g., Daikyo Seiko, Ltd. or West Pharmaceutical Services, Inc. The steps described herein are conducted, in many aspects, in a lyophilization chamber or ice nucleation system. Lyophilization chambers may be run per the manufacturer's instructions suitable for pharmaceutical compositions. In various aspects of the disclosure, the lyophilization method does not comprise an annealing step. “Annealing” refers to a process in which the temperature of the formulation is cycled (e.g., from a low temperature to a higher temperature, and then back to the low temperature). Various aspects of the disclosed method allow production of a lyophilized product without such an annealing step.

The method of the disclosure comprises inducing ice nucleation in a liquid bispecific molecule composition in a vial (referred to herein as “ice nucleation step”). Ice nucleation may be initiated using any of a number of methods including, but not limited to, ice fog, sudden/rapid depressurization, and vacuum-induced evaporative cooling. Other methods of controlled ice nucleation include, e.g., ultrasound, gap freezing, electro freezing, temperature quench freezing, use of precooled shelf, and mechanical agitation.

In various aspects of the disclosure, ice nucleation is induced in the liquid bispecific molecule composition via ice fog. Ice fog involves “seeding” a supercooled solution in vials with externally-generated ice crystals. An ice fog generator is used to produce a fine ice crystal suspension which is injected into the lyophilizer chamber. The crystals from the ice fog serve as ice seeds to the supercooled liquid product in the vials. Once the ice crystals from the fog enter the partially stoppered vials and contact the surface of the supercooled liquid, ice nucleation occurs instantaneously within the vial at a specified shelf temperature. This occurs simultaneously across all vials, improving intra-batch homogeneity. Ice fog systems are available from IMA Life (Tonawanda, NY) and Millrock Technologies (Kingston, NY). Ice fog technology is further described in, e.g., Azzarella et al., BioPharm. Int., 29(12) (2017), 36-41.

In various aspects of the disclosure, ice nucleation is induced in the liquid bispecific molecule composition via depressurization. Rapid depressurization generally involves first pressurizing a lyophilizer chamber to 1.5 to 2 atmospheres (about 20-30 psig) using an inert gas, such as nitrogen, then rapidly (e.g., in 3 seconds or less) releasing the pressure to slightly above ambient. The rapid shift in pressure induces nucleation in the vials. Rapid depressurization systems are available from SP Scientific (Gardiner, NY) and further described in, e.g., Luoma et al., “Controlled Ice Nucleation Using ControLyo® Pressurization-Depressurization Method”, In: Ward K., Matejtschuk P. (eds) Lyophilization of Pharmaceuticals and Biologicals. Methods in Pharmacology and Toxicology. Humana Press, New York, NY, 2019, pp. 57-77.

Vacuum-induced evaporative cooling generally involves reducing lyophilization chamber pressure to just above the boiling point of the solution and allowing the enhanced evaporative cooling effect of the liquid surface to cause nucleation. Vacuum-induced evaporative cooling systems are available from HOF Sonderanlagenbau GmbH (Lohra, Germany).

Ice nucleation (e.g., via ice fog) is induced in the liquid bispecific molecule composition in the context of the present invention in a vial exposed to a first temperature of about −18° C. to about −10° C. (such as about −18° C., about −17° C., about −16° C., about −15° C., about −14° C., about −13° C., about −12° C., about −11° C., or about −10° C.,) for a time period of about 60 minutes to about five hours. Optionally, the ice nucleation step comprises exposing the vial to the first temperature for about 30 minutes to about two hours, e.g., about 90 minutes to about two hours. In various aspects, the vial is exposed to a first temperature of about −15° C. A higher temperature such as −7° C. leads to significantly higher percental levels of HMWS which is neither desired in the context of the present invention nor in the production of therapeutic bispecific molecules in general. Hence, the careful selection of specific temperature ranges for the CIN step is not arbitrary. Optionally, this step of the method further comprises holding the ice-nucleated bispecific molecule composition at the temperature for a post-nucleation period of time of up to two hours (optionally at the same temperature). For example, the post-nucleation hold time may be about 30 minutes to about 90 minutes (e.g., about 45 minutes to about 75 minutes, such as 50 minutes, 55 minutes, 60 minutes, 65 minutes, 70 minutes, or 75 minutes). In various aspects, the post-nucleation hold time is about 60 minutes.

The method further comprises exposing the vial to a second temperature of about −25° C. to about −50° C. for a second period of time (referred to herein as a “freeze step”). The second temperature may be, e.g., about −25° C. to about −45° C., about −25° C. to about −40° C., about −25° C. to about −35° C., about −35° C. to about −50° C., about −40° C. to about −50° C., or about −45° C. to about −50° C. (such as about −40° C., about −41° C., about −42° C., about −43° C., about −44° C., about −45° C., about −46° C., about −47° C., about −48° C., about −49° C., or about −50° C.). In various aspects, the freeze step comprises exposing the vial to a second temperature of about −45° C. The second period of time is optionally about one hour to about five hours, such as about two hours to about four hours. The second period of time may be about 60 minutes, about 90 minutes, about 120 minutes, about 150 minutes, about 180 minutes, about 210 minutes, or about 240 minutes (or any range comprising these endpoints), In various aspects, the second period of time is about three hours.

The rate of cooling the vials may be controlled, in various aspects of the disclosure. For example, the first temperature is optionally transitioned to the second temperature at a rate of about 0.01° C. to about 0.5° C. per minute (e.g., about 0.05° C. to about 0.45° C. per minute, about 0.1° C. to about 0.3° C. per minute, or about 0.15° C. to about 0.25° C. per minute). In various aspects, the first temperature is transitioned to the second temperature at a rate of about 0.2° C. per minute.

The method further comprises drying the composition resulting from the freeze step at a third temperature of about 0° C. to about 40° C. (referred to herein as a “drying step”), resulting in a vial comprising a lyophilized bispecific molecule composition having an aspect ratio of greater than or equal to about 0.75. In various aspects, the third temperature utilized in the drying step is about 0° C. to about 35° C., about 0° C. to about 30° C., about 5° C. to about 40° C., about 10° C. to about 40° C., about 15° C. to about 40° C., about 20° C. to about 40° C., about 25° C. to about 40° C., about 30° C. to about 40° C., or about 35° C. to about 40° C. Optionally, the third temperature is about 0° C. to about 25° C., such as about 10° C. to about 25° C. (e.g., about 25° C.).

Optionally, the transition of the freeze step to the drying step comprises increasing the temperature at rate of about 0.2° C. to about 0.7° C. per minute and holding the vial at a temperature of about −40° C. to about −30° C. for about 15 minutes to about an hour. For example, the transition may comprise increasing the temperature at a rate of about 0.2° C., 0.3° C., 0.4° C., 0.5° C., 0.6° C., or 0.7° C. The transition may occur over a time period of, e.g., about 15 minutes, 30 minutes, 45 minutes, or 60 minutes.

The drying step may, in various aspects of the disclosure, comprise exposing the vial comprising the bispecific molecule composition to an increase in temperature at rate of about 0.01° C. to about 0.5° C. per minute (e.g., about 0.05° C. to about 0.45° C. per minute, about 0.1° C. to about 0.3° C. per minute, or about 0.15° C. to about 0.25° C. per minute). In various aspects of the disclosure, the drying step comprises (1) holding the vial at a temperature of about −5° C. to about 5° C. for about 8 hours to about 12 hours and (2) holding the vial at a temperature of about 20° C. to about 30° C. for about 20 hours to about 50 hours. In exemplary aspects of the disclosure, drying step 1 comprises holding the vial at a temperature of about −5° C. to about 0° C., about 0° C. to about 5° C., or about −2° C. to about 2° C.; such as about −5° C., about −4° C., about −3° C., about −2° C., about −1° C., about 0° C., about 1° C., about 2° C., about 3° C., about 4° C., about 5° C., or any range with these endpoints. The time period for drying step 1 is, in various aspects, about 8 hours to about 10 hours, about 9 hours to about 11 hours, or about 10 hours to about 12 hours; such as about 8 hours, about 9 hours, about 10 hours, about 11 hours, or about 12 hours, or any range with these endpoints. In exemplary aspects of the disclosure, drying step 2 comprises holding the vial at a temperature of about 25° C. to about 30° C., about 20° C. to about 25° C., or about 23° C. to about 27° C.; such as about 20° C., about 21° C., about 22° C., about 23° C., about 24° C., about 25° C., about 26° C., about 27° C., about 28° C., about 29° C., or about 30° C., or any range with these endpoints. The time period for drying step 2 is optionally about 20 hours to about 45 hours, about 20 hours to about 40 hours, about 20 hours to about 35 hours, about 20 hours to about 30 hours, about 25 hours to about 50 hours, about 30 hours to about 50 hours, about 35 hours to about 50 hours, about 40 hours to about 50 hours, or about 45 hours to about 50 hours (e.g., about 20 hours, about 25 hours, about 30 hours, about 35 hours, about 40 hours, about 45 hours, or about 50 hours). Drying step (1) optionally comprises holding the vial at a temperature of about 0° C. for about 10 hours. Drying step (2) optionally comprises holding the vial at a temperature of about 25° C. for about 40 hours.

The method of the disclosure permits use of higher volumes of liquid composition in the vials prior to lyophilization. In this regard, the liquid bispecific molecule composition may fill at least 50% of the vial volume prior to performing the ice nucleation step. The method may comprise, prior to the ice nucleation step, filling at least 50% of the volume of the vials with the liquid bispecific molecule composition. The liquid bispecific molecule composition may fill at least 55%, 60%, 65%, or 75% of the vial.

The disclosure further provides a lyophilized bispecific molecule composition prepared via the method described herein. The lyophilized bispecific molecule formulation optionally further comprises a saccharide, a surfactant, and/or a buffer. The formulation also optionally has a pH of about 3 to about 7 (or about 3.5, 4, 4.5, 5, 5.5, 6, 6.5, or 7). In some cases, the pH is about 4 to about 6. In some preferred cases the pH of the formulation is about 4, or about 4.2. In various cases, the pH of the formulation is about 5. In some embodiments, the pH of the formulation is about 6.

In some embodiments, the bispecific molecule of the lyophilized formulation is an antigen-binding protein. An “antigen-binding protein” is a protein comprising a domain that binds a specified target antigen (such as HER2 or CD3 and/or DLL3, BCMA, or CD33). An antigen-binding protein comprises a scaffold or framework portion that allows the antigen binding domain to adopt a conformation that promotes binding of the antigen-binding protein to the antigen.

The term “half-life” is understood herein as the time taken for concentration of a biological substance (such as a the bispecific molecule of the invention) to decrease from its maximum concentration (Cₘₐₓ) to half of Cₘₐₓ in the blood plasma. An extended half-life is preferably at least 40 hours, more preferably, 50, 60, 70, 80, 90, or 100 hours in comparison to a non-half-life extended bispecific molecule showing typically a half-life of 24 hours or less.

The term “polypeptide” is understood herein as an organic polymer which comprises at least one continuous, unbranched amino acid chain. In the context of the present invention, a polypeptide comprising more than one amino acid chain is likewise envisaged. An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The term “antigen-binding polypeptide” according to the present invention is preferably a polypeptide which immuno-specifically binds to its target or antigen. It typically comprises the heavy chain variable region (VH) and/or the light chain variable region (VL) of an antibody, or comprises domains derived therefrom. A polypeptide according to the invention comprises the minimum structural requirements of an antibody which allow for immuno-specific target binding. This minimum requirement may e.g. be defined by the presence of at least three light chain CDRs (i.e. CDR1, CDR2 and CDR3 of the VL region) and/or three heavy chain CDRs (i.e. CDR1, CDR2 and CDR3 of the VH region), preferably of all six CDRs. An antigen-binding molecule of the present invention is preferably a T-cell engaging polypeptide which may hence be characterized by the presence of three or six CDRs in either one or both binding domains, and the skilled person knows where (in which order) those CDRs are located within the binding domain. Preferably, an “antigen-binding molecule” is understood as an “antigen-binding polypeptide” in the context of the present invention. In an alternative embodiment, an antigen-binding polypeptide of the present invention may be an aptamer.

Alternatively, a molecule in the context of the present invention, is an antigen-binding polypeptide which corresponds to an “antibody construct” which typically refers to a molecule in which the structure and/or function is/are based on the structure and/or function of an antibody, e.g., of a full-length or whole immunoglobulin molecule. An antigen-binding molecule is hence capable of binding to its specific target or antigen and/or is/are drawn from the variable heavy chain (VH) and/or variable light chain (VL) domains of an antibody or fragment thereof. Furthermore, the domain which binds to its binding partner according to the present invention is understood herein as a binding domain of an antigen-binding molecule according to the invention. Typically, a binding domain according to the present invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. An alternative approach to define the minimal structure requirements of an antibody is the definition of the epitope of the antibody within the structure of the specific target, respectively, the protein domain of the target protein composing the epitope region (epitope cluster) or by reference to a specific antibody competing with the epitope of the defined antibody. The antibodies on which the constructs according to the invention are based include for example monoclonal, recombinant, chimeric, deimmunized, humanized and human antibodies.

In the context of the present invention, a polypeptide of the present invention binds to its respective target structure in a particular manner. Preferably, a polypeptide according to the present invention comprises one paratope per binding domain which specifically or immuno-specifically binds to”, “(specifically or immuno-specifically) recognizes”, or “(specifically or immuno-specifically) reacts with” its respective target structure. This means in accordance with this invention that a polypeptide or a binding domain thereof interacts or (immuno-)specifically interacts with a given epitope on the target molecule (antigen) and CD3, respectively. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, a binding domain that (immuno-) specifically binds to its target (such as a human target) may, however, cross-react with homologous target molecules from different species (such as, from non-human primates). The term “specific/immuno-specific binding” can hence include the binding of a binding domain to epitopes and/or structurally related epitopes in more than one species. The term “(immuno-) selectively binds” does exclude the binding to structurally related epitopes.

The binding domain of an antigen-binding molecule according to the invention may e.g. comprise the above referred groups of CDRs. Preferably, those CDRs are comprised in the framework of an antibody light chain variable region (VL) and an antibody heavy chain variable region (VH); however, it does not have to comprise both. Fd fragments, for example, have two VH regions and often retain some antigen-binding function of the intact antigen-binding domain. Additional examples for the format of antibody fragments, antibody variants or binding domains include (1) a Fab fragment, a monovalent fragment having the VL, VH, CL and CH1 domains; (2) a F(ab′)2 fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody, (5) a dAb fragment (Ward et al., (1989) Nature 341:544-546), which has a VH domain; (6) an isolated complementarity determining region (CDR), and (7) a single chain Fv (scFv), the latter being preferred (for example, derived from an scFV-library). Examples for embodiments of antigen-binding molecules according to the invention are e.g. described in WO 00/006605, WO 2005/040220, WO 2008/119567, WO 2010/037838, WO 2013/026837, WO 2013/026833, US 2014/0308285, US 2014/0302037, WO 2014/144722, WO 2014/151910, and WO 2015/048272.

Also, within the definition of “binding domain” or “domain which binds” are fragments of full-length antibodies, such as VH, VHH, VL, (s)dAb, Fv, Fd, Fab, Fab′, F(ab′)2 or “r IgG” (“half antibody”). Antigen-binding molecules according to the invention may also comprise modified fragments of antibodies, also called antibody variants, such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “multibodies” such as triabodies or tetrabodies, and single domain antibodies such as nanobodies or single variable domain antibodies comprising merely one variable domain, which may be VHH, VH or VL, that specifically bind an antigen or epitope independently of other V regions or domains. Typically, a binding domain of the present invention comprises a paratope which facilitates the binding to its binding partner.

As used herein, the terms “single-chain Fv,” “single-chain antibodies” or “scFv” refer to single polypeptide chain antibody fragments that comprise the variable regions from both the heavy and light chains, but lack the constant regions. Generally, a single-chain antibody further comprises a polypeptide linker between the VH and VL domains which enables it to form the desired structure which would allow for antigen binding. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, and/or humanized and/or synthetic.

In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a molecule according to the present invention is provided with a paratope comprising a set of 6 complementarity-determining regions (CDR loops) with three of each being comprised within the antibody-derived VH and VL sequence, respectively.

Furthermore, the definition of the term “antigen-binding molecule” includes preferably polyvalent/multivalent constructs and, thus, bispecific molecules, wherein bispecific means that they specifically bind to two cell types comprising distinctive antigenic structures, i.e. target cell(s) and effector cell(s). As the antigen-binding molecules of the present invention are preferably multitargeting, they are typically as well as polyvalent/multivalent molecules, i.e. they specifically bind more than two antigenic structures, preferably four distinct binding domains in the context of the present invention which are two target binding domains and two CD3 binding domains. The term “multitargeting bispecific antigen-binding molecule” comprises the terms “multitargeting bispecific T-cell engager molecule” and “multitargeting bispecific T-cell engager polypeptide (MBiTEP)”. A preferred “multitargeting bispecific antigen-binding molecule” is a “multitargeting bispecific T-cell engager molecule” or a “multitargeting bispecific T-cell engager polypeptide (MBiTEP)”. The term multitargeting bispecific T-cell engager molecule” is understood to comprise the term “multitargeting bispecific T-cell engager polypeptid. Moreover, the definition of the term “antigen-binding molecule” includes molecules comprising only one polypeptide chain as well as molecules consisting of more than one polypeptide chain, which chains can be either identical (homodimers, homotrimers or homo oligomers) or different (heterodimer, heterotrimer or heterooligomer). Such molecules comprising more than one polypeptide chain, i.e. typically two chains, have these chains typically attached to each other as heterodimers via charged pair binding, e.g. within a heteroFc entity which serves as a spacer and half-life extending moiety in between the two bispecific entities as described herein. Examples for the above identified antigen-binding molecules, e.g. antibody-based molecules and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “bispecific” as used herein refers to an antigen-binding molecule which is “at least bispecific”, i.e., it addresses two different cell types, i.e. target and effector cells, and comprises at least a first and third binding domain and a second and fourth binding domain, wherein at least two binding domains bind to two antigens or targets selected preferably from CD20, CD22, FLT3, MSLN, CDH3, CLL1 and EpCAM, and the other two binding domains of the same molecule bind to another antigen (here: CD3) on an effector cell, typically on a T cell. Accordingly, antigen-binding molecules according to the invention comprise specificities for at least two different antigens or targets. For example, two domains do preferably not bind to an extracellular epitope of CD3ε of one or more of the species as described herein.

The term “target cell surface antigen” refers to an antigenic structure expressed by a cell and which is present at the cell surface such that it is accessible for an antigen-binding molecule as described herein. A preferred target cell surface antigen in the context of the present invention is a tumor associated antigen (TAA). It may be a protein, preferably the extracellular portion of a protein, or a carbohydrate structure, preferably a carbohydrate structure of a protein, such as a glycoprotein. It is preferably a tumor antigen. The term “bispecific antigen-binding molecule” of the invention also encompasses bispecific multitargeting antigen-binding molecules such as tritargeting antigen-binding molecules, the latter ones including three binding domains, or constructs having more than three (e.g. four, five . . . ) specificities.

Preferred in the context of the present invention is a molecule which is “multitargeting”, which is understood herein to be “at least targeting two targets (e.g. TAAs) per molecule of the invention typically per target cell”. In this regard, a multitargeting molecule such as an antigen-binding molecule is specific for two—typically identical—effector structures on an effector cell such as CD3, more preferably CD3epsilon (CD3e, which is comprised whenever reference is made to the “CD3” in the present invention), and at least two target cell surface antigens. Said specificity is conferred by respective binding domains as defined herein. Typically, “multitargeting” refers to a molecule which is specific for at least two (preferably different) target cell surface antigens (e.g. TAAs) which confers preferred properties of a multitargeting antigen-binding molecule according to the present invention, namely mitigation of antigen loss and increase of selectivity, i.e. selectivity for killing target cells which co-express the targets for which the molecule of the invention has binding domains and which target cells are associated with a disease. Thereby, the therapeutic window of the molecule of the invention is increased with respect to monotargeting bispecific molecules which typically leads to higher drug tolerability as demonstrated herein.

A T-cell engaging antigen-binding molecule, e.g. a single chain polypeptide, according to the present invention is preferably bispecific which is understood herein to typically comprise one domain binding to at least one target antigen and another domain binding to CD3. Hence, it does not occur naturally, and it is markedly different in its function from naturally occurring products. A polypeptide in accordance with the invention is hence an artificial “hybrid” polypeptide comprising at least two distinct binding domains with different specificities and is, thus, bispecific. Bispecific antigen-binding molecules can be produced by a variety of methods including fusion of hybridomas or linking of Fab′ fragments. See, e.g., Songsivilai & Lachmann, Clin. Exp. Immunol. 79:315-321 (1990).

The at least four binding domains and the variable domains (VH/VL) of the antigen-binding molecule of the present invention typically comprise peptide linkers (spacer peptides). The term “peptide linker” comprises in accordance with the present invention an amino acid sequence by which the amino acid sequences of one (variable and/or binding) domain and another (variable and/or binding) domain of the antigen-binding molecule of the invention are linked with each other. The peptide linker between the first and the second binding domain and the third and the fourth domain, wherein the first and the third domain are preferably capable to bind simultaneously to two targets, which are preferably different targets (e.g. TAA1 and TAA2) preferably on the same cell, are preferably flexible and of limited length, e.g. of 5, 6, 7, 8, 9, 10, 11, 12, 13, 14, 15, 16, 17 or 18 amino acids. The peptide linkers can also be used to fuse the spacer to the other domains of the antigen-binding molecule of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antigen-binding molecule of the invention. However, typically the linker between the first and the second target binding domain differs from the intra-binder linker which links the VH and VL within the target binding domain. Said difference is the linker between the fist and the second binding domain having one amino acid more than intra-binder linkers, e.g. six and five amino acids, respectively, such as SGGGGS versus GGGGS. This confers surprisingly flexibility and stability at the same time in the specific antigen-binding molecule format as described herein. The spacer (or synonymously spacer entity) between the two bispecific entities as described herein is a specific embodiment of a linker because a spacer also functions as a linker because it contributes to linking the two bispecific entities to preferably build at least one continuous polypeptide chain comprising the four binding domains or parts thereof. However, in addition, the spacer functions as an entity which spaces the two bispecific entities sterically apart. Accordingly, a spacer in the context of the present invention is a specific embodiment of a linker which—together with two further short and flexible linkers on each end—contributes to linking the two binding domains (of two different bispecific entities) but first and foremost spaces them apart in such a way that the two bispecific entities can advantageously act as described herein, e.g. show a surprisingly high selectivity gap.

The antigen-binding molecules of the present invention are preferably “in vitro generated antigen-binding molecules”. This term refers to an antigen-binding molecule according to the above definition where all or part of the variable region (e.g., at least one CDR) is generated in a non-immune cell selection, e.g., an in vitro phage display, protein chip or any other method in which candidate sequences can be tested for their ability to bind to an antigen. This term thus preferably excludes sequences generated solely by genomic rearrangement in an immune cell in an animal. A “recombinant antibody” is an antibody made through the use of recombinant DNA technology or genetic engineering.

The term “monoclonal antibody” (mAb) or monoclonal antibody from which an antigen-binding molecule as used herein is derived refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). In addition to their specificity, the monoclonal antibodies are advantageous in that they are synthesized by the hybridoma culture, hence uncontaminated by other immunoglobulins. The modifier “monoclonal” indicates the character of the antibody as being obtained from a substantially homogeneous population of antibodies, and is not to be construed as requiring production of the antibody by any particular method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance analysis, e.g. Biacore™ to identify one or more hybridomas that produce an antibody that specifically binds with a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the Biacore system can be used to increase the efficiency of phage antibodies which bind to an epitope of a target cell surface antigen (Schier, Human Antibodies Hybridomas 7 (1996), 97-105; Malmborg, J. Immunol. Methods 183 (1995), 7-13).

Another exemplary method of making monoclonal antibodies includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified antigen-binding molecules include humanized variants of non-human antibodies, “affinity matured” antibodies (see, e.g. Hawkins et al. J. Mol. Biol. 254, 889-896 (1992) and Lowman et al., Biochemistry 30, 10832-10837 (1991)) and antibody mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antigen-binding molecules, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antigen-binding molecules involves substituting one or more hypervariable region residues of a parent antibody (e. g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e. g. 6-7 sides) are mutated to generate all possible amino acid substitutions at each side. The antibody variants thus generated are displayed in a monovalent fashion from filamentous phage particles as fusions to the gene Ill product of M13 packaged within each particle. The phage-displayed variants are then screened for their biological activity (e. g. binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and, e.g., human CS1, BCMA, CD20, CD22, FLT3, CD123, CDH3, MSLN, CLL1 or EpCAM. Such contact residues and neighbouring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antigen-binding molecules of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci, USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antigen-binding molecule, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol, Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, LA. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antigen-binding molecules, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

The term “human antibody”, “human antigen-binding molecule” and “human binding domain” includes antibodies, antigen-binding molecules and binding domains having antibody regions such as variable and constant regions or domains which correspond substantially to human germline immunoglobulin sequences known in the art, including, for example, those described by Kabat et al. (1991) (loc. cit.). The human antibodies, antigen-binding molecules or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antigen-binding molecules or binding domains can have at least one, two, three, four, five, or more positions replaced with an amino acid residue that is not encoded by the human germline immunoglobulin sequence. The definition of human antibodies, antigen-binding molecules and binding domains as used herein also contemplates fully human antibodies, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

In some embodiments, the antigen-binding molecules of the invention are “isolated” or “substantially pure” antigen-binding molecules. “Isolated” or “substantially pure”, when used to describe the antigen-binding molecules disclosed herein, means an antigen-binding molecule that has been identified, separated and/or recovered from a component of its production environment. Preferably, the antigen-binding molecule is free or substantially free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. The antigen-binding molecules may e.g. constitute at least about 5%, or at least about 50% by weight of the total protein in a given sample. It is understood that the isolated protein may constitute from 5% to 99.9% by weight of the total protein content, depending on the circumstances. The polypeptide may be made at a significantly higher concentration through the use of an inducible promoter or high expression promoter, such that it is made at increased concentration levels. The definition includes the production of an antigen-binding molecule in a wide variety of organisms and/or host cells that are known in the art. In preferred embodiments, the antigen-binding molecule will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated antigen-binding molecule will be prepared by at least one purification step.

The term “binding domain” characterizes in connection with the present invention a domain which (specifically) binds to/interacts with/recognizes a given target epitope or a given target side on the target molecules (antigens), e.g. CS1, BCMA, CD20, CD22, FLT3, CD123, CLL1, MSLN, or EpCAM, and CD3, respectively. The structure and function of the typically first and third or second and fourth binding domain (recognizing e.g. CS1, BCMA, CD20, CD22, FLT3, CD123, CLL1, MSLN, or EpCAM), and preferably also the structure and/or function of the effector binding domain (typically the second and fourth or first and third binding domain recognizing CD3), is/are based on the structure and/or function of an antibody, e.g. of a full-length or whole immunoglobulin molecule, and/or is/are drawn from the variable heavy chain (VH) and/or variable light chain (VL) domains of an antibody or fragment thereof. Preferably the target cell surface antigen(s) binding domain(s) is/are characterized by the presence of three light chain CDRs (i.e. CDR1, CDR2 and CDR3 of the VL region) and/or three heavy chain CDRs (i.e. CDR1, CDR2 and CDR3 of the VH region). The effector (typically CD3) binding domain preferably also comprises the minimum structural requirements of an antibody which allow for the target binding. More preferably, the second binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that the first and/or second binding domain is produced by or obtainable by phage-display or library screening methods rather than by grafting CDR sequences from a pre-existing (monoclonal) antibody into a scaffold.

According to the present invention, binding domains are in the form of one or more polypeptides. Such polypeptides may include proteinaceous parts and non-proteinaceous parts (e.g. chemical linkers or chemical crosslinking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a heteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

Preferably the binding domains which binds to any of CS1, BCMA, CD20, CD22, FLT3, CD123, CLL1, CDH3, MSLN, and EpCAM, and/or the binding domains which binds to CD31 is/are human binding domains. Antibodies and antigen-binding molecules comprising at least one human binding domain avoid some of the problems associated with antibodies or antigen-binding molecules that possess non-human such as rodent (e.g. murine, rat, hamster or rabbit) variable and/or constant regions. The presence of such rodent derived proteins can lead to the rapid clearance of the antibodies or antigen-binding molecules or can lead to the generation of an immune response against the antibody or antigen-binding molecule by a patient. In order to avoid the use of rodent derived antibodies or antigen-binding molecules, human or fully human antibodies/antigen-binding molecules can be generated through the introduction of human antibody function into a rodent so that the rodent produces fully human antibodies.

The term “High molecular weight species” (HMWS) typically refers to product-related variants of the bispecific molecules of the invention. HMWs may include dimers, trimers, tetramers, etc., formed of monomers that can be either covalently or non-covalently linked. HMWS may consist of misfolded monomers in which surfaces of the monomer are exposed that typically would not be in the monomeric form. They may or may not have an impact on safety and/or efficacy but are generally to be avoided from a regulatory perspective. Typically, HMWS in the context of the present invention are determined by Size Exclusion Chromatography (SE-UHPLC) and may represent a fraction different than the “main peak”, i.e. the desired monomeric product.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gin or Q); glutamic acid (Giu or E); glycine (Giy or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Giu); a positively charged sidechain (e.g., Arg, His, Lys); or an uncharged polar side chain (e.g., Asn, Cys, Gin, Giy, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antibody construct of the invention. An insertional variant of the antibody construct of the invention includes the fusion to the N-terminus or to the C-terminus of the antibody construct of an enzyme or the fusion to a polypeptide.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g. charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as the target cell surface antigen or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody construct may have different degrees of identity to their substituted sequences, e.g., CDRL1 may have 80%, while CDRL3 may have 90%.

Preferred substitutions (or replacements) are conservative substitutions. However, any substitution (including non-conservative substitution or one or more from the “exemplary substitutions” listed in Table 3, below) is envisaged as long as the antibody construct retains its capability to bind to the target cell surface antigen via the first domain and to CD3, respectively CD3 epsilon, via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Conservative substitutions are shown in Table 3 under the heading of “preferred substitutions”. If such substitutions result in a change in biological activity, then more substantial changes, denominated “exemplary substitutions” in Table 3, or as further described below in reference to amino acid classes, may be introduced and the products screened for a desired characteristic.

TABLE 1
Amino acid substitutions

| | | Preferred |
|---|---|---|
| Original | Exemplary Substitutions | Substitutions |
| Ala (A) | val, leu, ile | Val |
| Arg I | lys, gln, asn | Lys |
| Asn (N) | gln, his, asp, lys, arg | Gln |
| Asp (D) | glu, asn | Glu |
| Cys I | ser, ala | Ser |
| Gln (Q) | asn, glu | Asn |
| Glu I | asp, gln | Asp |
| Gly (G) | Ala | Ala |
| His (H) | asn, gln, lys, arg | Arg |
| Ile (I) | leu, val, met, ala, phe | Leu |
| Leu (L) | norleucine, ile, val, met, ala | Ile |
| Lys (K) | arg, gln, asn | Arg |
| Met (M) | leu, phe, ile | Leu |
| Phe (F) | leu, val, ile, ala, tyr | Tyr |
| Pro (P) | Ala | Ala |
| Ser (S) | Thr | Thr |
| Thr (T) | Ser | Ser |
| Trp (W) | tyr, phe | Tyr |
| Tyr (Y) | trp, phe, thr, ser | Phe |
| Val (V) | ile, leu, met, phe, ala | Leu |

Substantial modifications in the biological properties of the antibody construct of the present invention are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe.

Non-conservative substitutions will entail exchanging a member of one of these classes for another class. Any cysteine residue not involved in maintaining the proper conformation of the antibody construct may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

For amino acid sequences, sequence identity and/or similarity is determined by using standard techniques known in the art, including, but not limited to, the local sequence identity algorithm of Smith and Waterman, 1981, Adv. Appl. Math, 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., 1984, Nucl. Acid Res. 12:387-395, preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360; the method is similar to that described by Higgins and Sharp, 1989, CABIOS 5:151-153. Useful PILEUP parameters including a default gap weight of 3.00, a default gap length weight of 0.10, and weighted end gaps.

Another example of a useful algorithm is the BLAST algorithm, described in: Altschul et al., 1990, J. Mol. Biol. 215:403-410; Altschul et al., 1997, Nucleic Acids Res. 25:3389-3402; and Karin et al., 1993, Proc. Natl. Acad. Sci. U.S.A. 90:5873-5787. A particularly useful BLAST program is the WU-BLAST-2 program which was obtained from Altschul et al., 1996, Methods in Enzymology 266:460-480. WU-BLAST-2 uses several search parameters, most of which are set to the default values. The adjustable parameters are set with the following values: overlap span=1, overlap fraction=0.125, word threshold (T)=II. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., 1993, Nucl. Acids Res. 25:3389-3402. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to about 22 bits.

Generally, the amino acid homology, similarity, or identity between individual variant CDRs or VH/VL sequences are at least 60% to the sequences depicted herein, and more typically with preferably increasing homologies or identities of at least 65% or 70%, more preferably at least 75% or 80%, even more preferably at least 85%, 90%, 91%, 92%, 93%, 94%, 95%, 96%, 97%, 98%, 99%, and almost 100%. In a similar manner, “percent (%) nucleic acid sequence identity” with respect to the nucleic acid sequence of the binding proteins identified herein is defined as the percentage of nucleotide residues in a candidate sequence that are identical with the nucleotide residues in the coding sequence of the antibody construct. A specific method utilizes the BLASTN module of WU-BLAST-2 set to the default parameters, with overlap span and overlap fraction set to 1 and 0.125, respectively.

Generally, the nucleic acid sequence homology, similarity, or identity between the nucleotide sequences encoding individual variant CDRs or VH/VL sequences and the nucleotide sequences depicted herein are at least 60%, and more typically with preferably increasing homologies or identities of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99%, and almost 100%. Thus, a “variant CDR” or a “variant VH/VL region” is one with the specified homology, similarity, or identity to the parent CDR/VH/VL of the invention, and shares biological function, including, but not limited to, at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR or VH/VL.

In one embodiment, the percentage of identity to human germline of the antibody constructs according to the invention is ≥70% or ≥75%, more preferably ≥80% or ≥85%, even more preferably ≥90%, and most preferably ≥91%, ≥92%, ≥93%, ≥94%, ≥95% or even ≥96%. Identity to human antibody germline gene products is thought to be an important feature to reduce the risk of therapeutic proteins to elicit an immune response against the drug in the patient during treatment. Hwang & Foote (“Immunogenicity of engineered antibodies”; Methods 36 (2005) 3-10) demonstrate that the reduction of non-human portions of drug antibody constructs leads to a decrease of risk to induce anti-drug antibodies in the patients during treatment. By comparing an exhaustive number of clinically evaluated antibody drugs and the respective immunogenicity data, the trend is shown that humanization of the V-regions of antibodies makes the protein less immunogenic (average 5.1% of patients) than antibodies carrying unaltered non-human V regions (average 23.59% of patients). A higher degree of identity to human sequences is hence desirable for V-region based protein therapeutics in the form of antibody constructs. For this purpose of determining the germline identity, the V-regions of VL can be aligned with the amino acid sequences of human germline V segments and J segments (http://vbase.mrc-cpe.cam.ac.uk/) using Vector NTI software and the amino acid sequence calculated by dividing the identical amino acid residues by the total number of amino acid residues of the VL in percent. The same can be for the VH segments (http://vbase.mrc-cpe.cam.ac.uk/) with the exception that the VH CDR3 may be excluded due to its high diversity and a lack of existing human germline VH CDR3 alignment partners. Recombinant techniques can then be used to increase sequence identity to human antibody germline genes.

In a further embodiment, the bispecific antibody constructs of the present invention exhibit high monomer yields under standard research scale conditions, e.g., in a standard two-step purification process. Preferably the monomer yield of the antibody constructs according to the invention is ≥0.25 mg/L supernatant, more preferably ≥0.5 mg/L, even more preferably ≥1 mg/L, and most preferably ≥3 mg/L supernatant.

Likewise, the yield of the dimeric antibody construct isoforms and hence the monomer percentage (i.e., monomer:(monomer+dimer)) of the antibody constructs can be determined. The productivity of monomeric and dimeric antibody constructs and the calculated monomer percentage can e.g. be obtained in the SEC purification step of culture supernatant from standardized research-scale production in roller bottles. In one embodiment, the monomer percentage of the antibody constructs is ≥80%, more preferably ≥85%, even more preferably ≥90%, and most preferably ≥95%.

In one embodiment, the antibody constructs have a preferred plasma stability (ratio of EC50 with plasma to EC50 w/o plasma) of ≤5 or ≤4, more preferably ≤3.5 or ≤3, even more preferably ≤2.5 or ≤2, and most preferably ≤1.5 or ≤1. The plasma stability of an antibody construct can be tested by incubation of the construct in human plasma at 37° C. for 24 hours followed by EC50 determination in a ⁵¹chromium release cytotoxicity assay. The effector cells in the cytotoxicity assay can be stimulated enriched human CD8 positive T cells. Target cells can e.g. be CHO cells transfected with the human target cell surface antigen. The effector to target cell (E:T) ratio can be chosen as 10:1. The human plasma pool used for this purpose is derived from the blood of healthy donors collected by EDTA coated syringes. Cellular components are removed by centrifugation and the upper plasma phase is collected and subsequently pooled. As control, antibody constructs are diluted immediately prior to the cytotoxicity assay in RPMI-1640 medium. The plasma stability is calculated as ratio of EC50 (after plasma incubation) to EC50 (control).

It is furthermore preferred that the monomer to dimer conversion of antibody constructs of the invention is low. The conversion can be measured under different conditions and analyzed by high performance size exclusion chromatography. For example, incubation of the monomeric isoforms of the antibody constructs can be carried out for 7 days at 37° C. and concentrations of e.g. 100 μg/ml or 250 μg/ml in an incubator. Under these conditions, it is preferred that the antibody constructs of the invention show a dimer percentage that is ≤5%, more preferably ≤4%, even more preferably ≤3%, even more preferably ≤2.5%, even more preferably ≤2%, even more preferably ≤1.5%, and most preferably ≤1% or ≤0.5% or even 0%.

It is also preferred that the bispecific antibody constructs of the present invention present with very low dimer conversion after a number of freeze/thaw cycles. For example, the antibody construct monomer is adjusted to a concentration of 250 μg/ml e.g. in generic formulation buffer and subjected to three freeze/thaw cycles (freezing at −80° C. for 30 min followed by thawing for 30 min at room temperature), followed by high performance SEC to determine the percentage of initially monomeric antibody construct, which had been converted into dimeric antibody construct. Preferably the dimer percentages of the bispecific antibody constructs are ≤5%, more preferably ≤4%, even more preferably ≤3%, even more preferably ≤2.5%, even more preferably ≤2%, even more preferably ≤1.5%, and most preferably ≤1% or even ≤0.5%, for example after three freeze/thaw cycles.

The bispecific antibody constructs of the present invention preferably show a favorable thermostability with aggregation temperatures ≥45° C. or ≥50° C., more preferably ≥52° C. or ≥54° C., even more preferably ≥56° C. or ≥57° C., and most preferably ≥58° C. or ≥59° C. The thermostability parameter can be determined in terms of antibody aggregation temperature as follows: Antibody solution at a concentration 250 μg/ml is transferred into a single use cuvette and placed in a Dynamic Light Scattering (DLS) device. The sample is heated from 40° C. to 70° C. at a heating rate of 0.5° C./min with constant acquisition of the measured radius. Increase of radius indicating melting of the protein and aggregation is used to calculate the aggregation temperature of the antibody.

Alternatively, temperature melting curves can be determined by Differential Scanning Calorimetry (DSC) to determine intrinsic biophysical protein stabilities of the antibody constructs. These experiments are performed using a MicroCal LLC (Northampton, MA, U.S.A) VP-DSC device. The energy uptake of a sample containing an antibody construct is recorded from 20° C. to 90° C. compared to a sample containing only the formulation buffer. The antibody constructs are adjusted to a final concentration of 250 μg/ml e.g. in SEC running buffer. For recording of the respective melting curve, the overall sample temperature is increased stepwise. At each temperature T energy uptake of the sample and the formulation buffer reference is recorded. The difference in energy uptake Cp (kcal/mole/° C.) of the sample minus the reference is plotted against the respective temperature. The melting temperature is defined as the temperature at the first maximum of energy uptake.

The target cell surface antigen×CD3 bispecific antibody constructs of the invention are also envisaged to have a turbidity (as measured by OD340 after concentration of purified monomeric antibody construct to 2.5 mg/ml and over night incubation) of ≤0.2, preferably of ≤0.15, more preferably of ≤0.12, even more preferably of ≤0.1, and most preferably of ≤0.08.

It is furthermore envisaged that the bispecific antibody constructs of the present invention exhibit therapeutic efficacy or anti-tumor activity. This can e.g. be assessed in a study as disclosed in the following example of an advanced stage human tumor xenograft model:

The skilled person knows how to modify or adapt certain parameters of this study, such as the number of injected tumor cells, the site of injection, the number of transplanted human T cells, the amount of bispecific antibody constructs to be administered, and the timelines, while still arriving at a meaningful and reproducible result. Preferably, the tumor growth inhibition T/C [%] is ≤70 or ≤60, more preferably ≤50 or ≤40, even more preferably ≤30 or ≤20 and most preferably ≤10 or ≤5 or even ≤2.5.

In a preferred embodiment of the antibody construct of the invention the antibody construct is a single chain antibody construct.

Also in a preferred embodiment of the antibody construct of the invention said third domain comprises in an amino to carboxyl order:
hinge-CH2-CH3-linker-hinge-CH2-CH3.

Also in one embodiment of the invention the CH2 domain of one or preferably each (both) polypeptide monomers of the third domain comprises an intra domain cysteine disulfide bridge. As known in the art the term “cysteine disulfide bridge” refers to a functional group with the general structure R—S—S—R. The linkage is also called an SS-bond or a disulfide bridge and is derived by the coupling of two thiol groups of cysteine residues. It is particularly preferred for the antibody construct of the invention that the cysteines forming the cysteine disulfide bridge in the mature antibody construct are introduced into the amino acid sequence of the CH2 domain corresponding to 309 and 321 (Kabat numbering).

In one embodiment of the invention a glycosylation site in Kabat position 314 of the CH2 domain is removed. It is preferred that this removal of the glycosylation site is achieved by a N314X substitution, wherein X is any amino acid excluding Q. Said substitution is preferably a N314G substitution. In a more preferred embodiment, said CH2 domain additionally comprises the following substitutions (position according to Kabat) V321C and R309C (these substitutions introduce the intra domain cysteine disulfide bridge at Kabat positions 309 and 321).

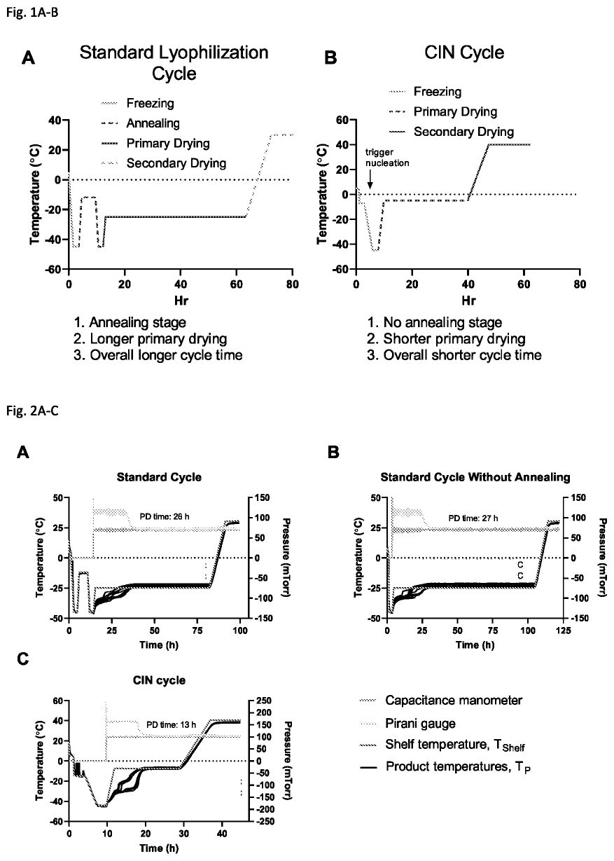

It is assumed that the preferred features of the antibody construct of the invention compared e.g. to the bispecific heteroFc antibody construct known in the art (FIG. 1b) may be inter alia related to the introduction of the above described modifications in the CH2 domain. Thus, it is preferred for the construct of the invention that the CH2 domains in the third domain of the antibody construct of the invention comprise the intra domain cysteine disulfide bridge at Kabat positions 309 and 321 and/or the glycosylation site at Kabat position 314 is removed by a N314X substitution as above, preferably by a N314G substitution.

In a further preferred embodiment of the invention the CH2 domains in the third domain of the antibody construct of the invention comprise the intra domain cysteine disulfide bridge at Kabat positions 309 and 321 and the glycosylation site at Kabat position 314 is removed by a N314G substitution.

In one embodiment the invention provides an antibody construct, wherein:
(i) the first domain comprises two antibody variable domains and the second domain comprises two antibody variable domains;
(ii) the first domain comprises one antibody variable domain and the second domain comprises two antibody variable domains;
(iii) the first domain comprises two antibody variable domains and the second domain comprises one antibody variable domain; or
(iv) the first domain comprises one antibody variable domain and the second domain comprises one antibody variable domain.

Accordingly, the first and the second domain may be binding domains comprising each two antibody variable domains such as a VH and a VL domain. Examples for such binding domains comprising two antibody variable domains where described herein above and comprise e.g. Fv fragments, scFv fragments or Fab fragments described herein above. Alternatively either one or both of those binding domains may comprise only a single variable domain. Examples for such single domain binding domains where described herein above and comprise e.g. nanobodies or single variable domain antibodies comprising merely one variable domain, which might be VHH, VH or VL, that specifically bind an antigen or epitope independently of other V regions or domains.

In a preferred embodiment of the antibody construct of the invention first and second domain are fused to the third domain via a peptide linker. Preferred peptide linker have been described herein above and are characterized by the amino acid sequence Gly-Gly-Gly-Gly-Ser, i.e. Gly₄Ser (SEQ ID NO: 187), or polymers thereof, i.e. (Gly₄Ser)x, where x is an integer of 1 or greater (e.g. 2 or 3). A particularly preferred linker for the fusion of the first and second domain to the third domain is depicted in SEQ ID Nos: 1.

In a preferred embodiment the antibody construct of the invention is characterized to comprise in an amino to carboxyl order:
(a) the first domain;
(b) a peptide linker having an amino acid sequence selected from the group consisting of SEQ ID Nos: 187-189;
(c) the second domain;
(d) a peptide linker having an amino acid sequence selected from the group consisting of SEQ ID NO: 187, 188, 189, 195, 196, 197 and 198;
(e) the first polypeptide monomer of the third domain;
(f) a peptide linker having an amino acid sequence selected from the group consisting of SEQ ID Nos: 191, 192, 193 and 194; and
(g) the second polypeptide monomer of the third domain.

In one aspect of the invention the target cell surface antigen bound by the first domain is a tumor antigen, an antigen specific for an immunological disorder or a viral antigen. The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by the normal ones. Tumor antigens can be exclusively expressed on tumor cells or might represent a tumor specific mutation compared to normal cells. In this case, they are called tumor-specific antigens. More common are antigens that are presented by tumor cells and normal cells, and they are called tumor-associated antigens. These tumor-associated antigens can be overexpressed compared to normal cells or are accessible for antibody binding in tumor cells due to the less compact structure of the tumor tissue compared to normal tissue. Non-limiting examples of tumor antigens as used herein are CDH19, MSLN, DLL3, FLT3, EGFRvIII, CD33, CD19, MUC17, CLDN18.2, CDH3, CD70, BCMA and PSMA.

In a preferred embodiment of the antibody construct of the invention the tumor antigen, preferably tumor antigen, is selected from the group consisting of CDH19, MSLN, DLL3, FLT3, EGFRvIII, CD33, CD19, MUC17, CLDN18.2, CDH3, CD70, BCMA and PSMA.

In one aspect of the invention the antibody construct comprises in an amino to carboxyl order:
(a) the first domain having an amino acid sequence selected from the group consisting of SEQ ID Nos: 7, 8, 17, 27, 28, 37, 38, 39, 40, 41, 48, 49, 50, 51, 52, 59, 60, 61, 62, 63, 64, 71, 72, 73, 74, 75. 76, 77, 78, 79, 80, 81, 89, 90, 91, 92, 93, 100, 101, 102, 103, 104, 113, 114, 121, 122,123, 124, 125, 131, 132, 133, 134, 135, 136, 143, 144, 145, 146, 147, 148, 149, 150, 151, 158,159, 160, 161, 162, 163, 164, 165, 166, 173, 174, 175, 176,177, 178, 179, 180, 181, 223, 235 and 246,
(b) a peptide linker having an amino acid sequence selected from the group consisting of SEQ ID Nos: 187-189;
(c) the second domain having an amino acid sequence selected from the group consisting of SEQ ID Nos: SEQ ID Nos: 23, 25, 41, 43, 59, 61, 77, 79, 95, 97, 113, 115, 131, 133, 149, 151, 167, 169, 185 or 187 of WO 2008/119567 or of SEQ ID NO: 202;
(d) a peptide linker having an amino acid sequence selected from the group consisting of SEQ ID Nos: 187, 188, 189, 195, 196, 197 and 198;
(e) the first polypeptide monomer of the third domain having a polypeptide sequence selected from the group consisting of SEQ ID Nos: 249-256;
(f) a peptide linker having an amino acid sequence selected from the group consisting of SEQ ID Nos: 191, 192, 193 and 194; and
(g) the second polypeptide monomer of the third domain having a polypeptide sequence selected from the group consisting of SEQ ID Nos: 249-256.

In one aspect, the bispecific antibody construct of the invention is characterized by having an amino acid sequence selected from the group consisting of and being directed to the respective target cell surface antigen:

| | | |
|---|---|---|
| (a) | SEQ ID Nos: 27, 28, 37 to 41; | CD33 |
| (b) | SEQ ID Nos: each of 48 to 52; | EGFRvIII |
| (c) | SEQ ID Nos: each of 59 to 64; | MSLN |
| (d) | SEQ ID Nos: each of 71 to 82 | CDH19 |
| (e) | SEQ ID Nos: each of 100 to 104 | DLL3 |
| (f) | SEQ ID Nos: 7, 8, 17, 113 and 114 | CD19 |
| (g) | SEQ ID Nos: each of 89 to 93 | FLT3 |
| (h) | SEQ ID Nos: each of 121 to 125 | CDH3 |
| (i) | SEQ ID Nos: each of 132 to 136 | BCMA |
| (j) | SEQ ID Nos: each of 143 to 151, | PSMA |
| | 158 to 166 and 173 to 181 | |
| (k) | SEQ ID NO 213 | MUC17 |
| (l) | SEQ ID NOs: each of 225 and 237 | CLDN18.2 and |
| (m) | SEQ ID No: 248 | CD70 |

In some embodiments, the bispecific antibody construct comprises a first binding domain that binds DLL3 comprising an anti-DLL3 variable light domain comprising.

In some embodiments, the bispecific antibody construct comprises a first binding domain that binds BCMA comprising an anti-BCMA variable light domain.

In some embodiments, the bispecific antibody construct comprises a first binding domain that binds CD33 comprising an anti-CD33 variable light domain.

In some aspects of the disclosure, the protein, such an antibody or bispecific antibody construct, is present in the liquid formulation (before lyophilization) in an amount ranging from about 0.1 mg/mL to about 100 mg/mL (or about 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, or 100 mg/mL). For example, the protein is optionally present in the liquid formulation in an amount ranging from about 0.1 mg/mL to about 70 mg/mL. In some cases, the protein is present in the liquid formulation in an amount ranging from about 0.5 mg/mL to about 30 mg/mL (or about 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, or 30 mg/mL). In various cases, the protein is present in the liquid formulation in an amount ranging from about 1 mg/mL to about 20 mg/mL (or about 1 mg/mL, 1.5 mg/mL, 2 mg/mL, 2.5 mg/mL, 3 mg/mL, 3.5 mg/mL, 4 mg/mL, 4.5 mg/mL, 5 mg/mL, 5.5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, 15 mg/mL, 15.5 mg/mL, 16 mg/mL, 16.5 mg/mL, 17 mg/mL, 17.5 mg/mL, 18 mg/mL, 18.5 mg/mL, 19 mg/mL, 19.5 mg/mL. or 20 mg/mL). In some aspects, the protein is present in the liquid formulation in an amount of about 21 mg/mL.

The protein formulation of the disclosure optionally comprises a saccharide. In some embodiments, the saccharide is a monosaccharide or a disaccharide. Suitable saccharides include, for example, glucose, galactose, fructose, xylose, sucrose, lactose, maltose, trehalose, or any combination thereof. In some cases, the saccharide comprises trehalose.

In some aspects, the liquid formulation (before lyophilization) comprises saccharide at a concentration of about 1% to about 15% w/v, or about 4% to about 13% w/v, or about 6% to about 12% w/v. In some embodiments, the liquid formulation comprises saccharide at a concentration of at least 1%, at least 2%, at least 3%, at least 4%, at least 5%, at least 6%, at least 7%, at least 8%, at least 9%, at least 10%, at least 11%, at least 12%, at least 13%, or at least 14% w/v. In some embodiments, the liquid formulation comprises saccharide at a concentration of about 1%, about 2%, about 3%, about 4%, about 5%, about 6%, about 7%, about 8%, about 9%, about 10%, about 11%, about 12%, about 13%, about 14%, or about 15% w/v. In some embodiments, the liquid formulation comprises saccharide at a concentration of about 7%, about 7.5%, about 8%, about 8.5%, about 9%, about 9.5%, about 10%, about 10.5%, about 11%, about 11.5%, or about 12% w/v. In some embodiments, the liquid formulation comprises saccharide at a concentration of about 7% to about 12% w/v. In some aspects, the liquid formulation comprises saccharide at a concentration of about 9% w/v. In some embodiments, the saccharide is sucrose and is present in the liquid formulation at a concentration ranging from about 6% to about 12% w/v.

The protein formulation of the disclosure optionally comprises a surfactant. Suitable surfactants include a polysorbate, a poloxomer, a polyoxyethylene, or any combination thereof. Contemplated surfactants include polysorbate 20, polysorbate 40, polysorbate 60, polysorbate 80, poloxamer 188, poloxamer 407, triton X-100, polyoxyethylene, PEG 3350, PEG 4000, and any combination thereof. In some aspects, the surfactant comprises a polysorbate. In some cases, the surfactant is polysorbate 20.

The protein formulations described herein can comprise one surfactant or a mixture of surfactants (although this is not required). In some aspects, the liquid formulation (before lyophilization) comprises a surfactant at a concentration of about 0.001% to about 5% w/v (or about 0.001% to about 0.5%, or about 0.004 to about 0.5% w/v or about 0.001 to about 0.01% w/v or about 0.004 to about 0.01% w/v). In some aspects, the liquid formulation comprises a surfactant at a concentration of at least 0.001, at least 0.002, at least 0.003, at least 0.004, at least 0.005, at least 0.007, at least 0.01, at least 0.05, at least 0.1, at least 0.2, at least 0.3, at least 0.4, at least 0.5, at least 0.6, at least 0.7, at least 0.8, at least 0.9, at least 1.0, at least 1.5, at least 2.0, at least 2.5, at least 3.0, at least 3.5, at least 4.0, or at least 4.5% w/v. In some aspects, the liquid formulation comprises a surfactant at a concentration of about 0.001% to about 0.5% w/v. In some aspects, the liquid formulation comprises a surfactant at a concentration of about 0.001 to about 0.01% w/v. In some aspects, the liquid formulation comprises a surfactant at a concentration of about 0.001 to about 0.01% w/v. In some aspects, the liquid formulation comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, to about 0.5% w/v. In some aspects, the liquid formulation comprises a surfactant at a concentration of about 0.001% to about 0.01% w/v.

The protein formulation of the disclosure optionally comprises a buffer. Suitable buffers include acetate buffers, glutamate buffers, citrate buffers, lactate buffers, succinate buffers, tartrate buffers, fumarate buffers, maleate buffers, histidine buffers, phosphate buffers, 2-(N-morpholino)ethanesulfonate buffers, or any combination thereof. In some cases, the buffer comprises histidine.

Buffering agents are often employed to control pH in the formulation. In some aspects, the buffer is added in a concentration that maintains pH of the liquid formulation of about 3 to about 7, or about 4 to about 6, about 4 to 5, or about 5 to about 6, or about 6 to about 6.5. The effect of pH on formulations may be characterized using any one or more of several approaches such as accelerated stability studies and calorimetric screening studies (Remmele R. L. Jr., et al., Biochemistry, 38(16): 5241-7 (1999)).

The buffer system (when present in the protein formulation) is selected to be physiologically compatible and to maintain a desired pH. The buffer may be present in the liquid formulation (before lyophilization) at a concentration between about 0.1 mM and about 1000 mM (1 M), or between about 5 mM and about 200 mM, or between about 5 mM to about 100 mM, or between about 10 mM and 50 about mM. Suitable buffer concentrations encompass concentrations of about 200 mM or less. In some aspects, the buffer in the liquid protein formulation (before lyophilization) is present in a concentration of about 190 mM, about 180 mM, about 170 mM, about 160 mM, about 150 mM, about 140 mM, about 130 mM, about 120 mM, about 110 mM, about 100 mM, about 80 mM, about 70 mM, about 60 mM, about 50 mM, about 40 mM, about 30 mM, about 20 mM, about 10 mM or about 5 mM. In some aspects, the concentration of the buffer is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, or 900 mM. In some aspects, the concentration of the buffer is between 1, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, or 90 mM and 100 mM. In some aspects, the concentration of the buffer is between 5, 6, 7, 8, 9, 10, 11, 12, 13, 14, 15, 16, 17, 18, 19, 20, 30, or 40 mM and 50 mM.

As an additional aspect, kits are provided which comprise a lyophilized protein composition described herein packaged in a manner which facilitates administration to subjects. In one aspect, the kit includes a lyophilized protein composition described herein packaged in a container such as a sealed bottle, vessel, single-use or multi-use vial, prefilled device (e.g. syringe), or prefilled injection device, optionally with a label affixed to the container or included in the package that describes use of the lyophilized protein composition. In one aspect, the pharmaceutical composition is packaged in a unit dosage form. The kit may include a device suitable for administering the reconstituted protein composition according to a specific route of administration, although this is not required. For example, the disclosure provides a dual chamber device for delivering a reconstituted protein composition disclosed herein to a subject in need thereof. Dual chamber devices are combination products containing the lyophilized protein composition disclosed herein and a diluent in two separate chambers of the device. Prefilled dual chamber devices are combination products containing freeze-dried drug and diluent in two separate chambers of the device. Suitable dual chamber devices for use with the instant disclosure are described in the art. See for example, Ingle R., Fang W. (2021). Int. Journal of Pharmaceutics 597, 12031.

The formulation described herein is useful as a pharmaceutical formulation in the treatment or amelioration of cancer in a subject in need thereof. The terms “subject in need” or those “in need of treatment” include subjects already afflicted with the disorder, as well as those in which the disorder is to be prevented. The “subject in need” or “patient” includes human and other mammalian subjects that receive either prophylactic or therapeutic treatment. “Treatment” does not require complete remission or eradication of the disease; any improvement in the disease and/or improvement in the symptoms associated with the disease are contemplated. For example, a therapeutic response would refer to one or more of the following improvements in the disease: (1) a reduction in the number of neoplastic cells; (2) an increase in neoplastic cell death; (3) inhibition of neoplastic cell survival; (4) inhibition (i.e., slowing to some extent, preferably halting) of tumor growth or appearance of new lesions; (5) slowing of disease progression; (6) an increased patient survival rate; (7) downgrade of stage of a cancer (e.g., Stage 2 to Stage 1); and/or (8) some relief from one or more symptoms associated with the disease or condition. The composition may also be administered to achieve disease prevention or slowing of onset, e.g., the avoidance of an occurrence or re-occurrence a tumor or cancer. Disease state is monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels (e.g., carcinoembryonic antigen (CEA)), cytology, histology, tumor biopsy sampling, and/or counting of tumor cells in circulation. These methods also are typically used to diagnose and stage cancer.

The disclosure provides a method of treating cancer, comprising administering to a subject in need thereof a therapeutically effective amount of a reconstituted composition based on the lyophilized formulation described herein. In certain embodiments, the subject is a human. In certain aspects, the cancer is a solid tumor. In some embodiments, the cancer is brain cancer, bladder cancer, breast cancer (e.g., triple negative breast cancer), clear cell kidney cancer, cervical cancer, colon and rectal cancer, endometrial cancer, gastric cancer, head/neck squamous cell carcinoma, lip and oral cancer, liver cancer, lung squamous cell carcinoma, melanoma, mesothelioma, non-small-cell lung cancer (NSCLC), non-melanoma skin cancer, ovarian cancer, oral cancer, pancreatic cancer, prostate cancer, neuroendocrine prostate cancer, renal cell carcinoma, sarcoma, small-cell lung cancer (SCLC), Squamous Cell Carcinoma of the Head and Neck (SCCHN), or thyroid cancer. In some aspects, the cancer is acute lymphoblastic leukemia (ALL), acute myeloid leukemia (AML), chronic lymphocytic leukemia (CLL), or chronic myeloid leukemia (CML). In some aspects, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL). The disclosure also provides use of a reconstituted composition based on the lyophilized formulation in a method of treating cancer, as well as use of the lyophilized formulation in the preparation of a medicament for treating cancer.

Preferably, the pharmaceutical formulation is administered parenterally, e.g., intravenously, subcutaneously, intratumorally, or intramuscularly. Parenteral administration may be achieved by injection, such as bolus injection, or by infusion, such as continuous infusion. Administration may be achieved via depot for long-term release. In some embodiments, the formulation is administered intravenously by an initial bolus followed by a continuous infusion to maintain therapeutic circulating levels of drug product. In some embodiments, the formulation is administered as a one-time dose. Pharmaceutical formulations may be administered using a medical device. Examples of medical devices for administering pharmaceutical formulations are described in U.S. Pat. Nos. 4,475,196; 4,439,196; 4,447,224; 4,447, 233; 4,486,194; 4,487,603; 4,596,556; 4,790,824; 4,941,880; 5,064,413; 5,312,335; 5,312,335; 5,383,851; and 5,399,163.

The Examples below illustrate representative features of the disclosure. From the description of these aspects, other aspects of the invention can be made and/or practiced based on the description provided below. The methods involve use of molecular biological techniques described in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. The example serves only to illustrate the invention and is not intended to limit the scope of the invention in any way.

### EXAMPLE

This example describes a process for preparing a lyophilized bispecific molecule composition according to an alternative method of including an annealing step and according to the present invention of including a CIN step, the latter resulting in a desired product property of low percental HMWS and desirable process time.

The use of annealing and controlled ice nucleation are two ways to address challenges associated with lyophilization, including aggregation and long processing times. Annealing and controlled ice nucleation both address this problem by reducing ice heterogeneity by producing larger ice crystals, resulting in a porous cake matrix with lowered resistance to mass transfer during sublimation.

Annealing involves holding the product between the glass transition and eutectic melting temperatures after stochastic ice nucleation (FIG. 1A). This melts the ice crystals, the smallest of which completely disappear, leaving the larger ones behind. The subsequent freezing then further grows the large crystals. Larger ice crystals lead to larger pores in the freeze-dried cake, which correspond to lower resistance to vapor moving through the dried layer and faster primary drying. However, holding the product above the glass transition temperature can promote the formation of high molecular weight species (HMWS) for some products due to the increased mobility and proximity of proteins. This issue has been observed for bispecific molecules (e.g. BiTE® molecule) products with protein concentrations above 5 mg/mL. Additionally, annealing prolongs the freezing phase of lyophilization, which partially offsets the reduction in primary drying time.

Controlled ice nucleation (CIN) produces larger ice crystals using a different method. For example, one CIN technique involves the injection of seed crystals to trigger ice nucleation (FIG. 1B). This triggers nucleation while samples are only slightly supercooled, and an isothermal hold after nucleation can be used to grow the crystals further. The lesser degree of supercooling results in larger ice crystals than those that are created in stochastic nucleation, and a shorter isothermal hold may pose a lower risk of HMWS formation when compared to annealing. In this example, a single chain bispecific T cell engager molecule lyophilization cycle without a CIN step, with and without annealing was compared with a CIN lyophilization to evaluate bispecific molecule drug products with “high protein concentration” (defined in this example as 15 mg/mL or greater). BCMA×CD3 bispecific molecule (20 mg/mL) and DLL3×CD3 bispecific molecule (15 mg/mL) were used as model high-concentration bispecific molecule products. Annealing is currently a typical option for lyophilized bispecific molecule drug products. Thus, such a lyophilization cycle was used as the representative annealing lyophilization cycle herein. The annealing step was removed from a lyophilization cycle to create a “cycle without annealing.” The CIN lyophilization cycle initially developed for the BCMA×CD3 bispecific molecule 1 mg 6R SKU7 was adapted for use with the high concentration bispecific molecule formulations. Lyophilization cycles were evaluated in terms of total cycle time and product quality.

### Materials and Methods

BCMA×CD3 bispecific molecule (20 mg/mL) and DLL3×CD3 bispecific molecule (15 mg/mL) were used for these experiments. Table 1 contains the details of the drug substances (DS) used.

TABLE 1
Bispecific molecule Drug Substances

| Material | Formulation |
|---|---|
| BCMAxCD3 bispecific | 20 mg/mL in L-glutamate, 9% w/v sucrose, |
| molecule | 0.01% w/v polysorbate 80, pH 4.2 |
| DLL3xCD3 bispecific | 15 mg/mL in L-glutamate, 9% w/v sucrose, |
| molecule | 0.01% w/v polysorbate 80, pH 4.2 |

BCMA×CD3 bispecific molecule and DLL3×CD3 bispecific molecule DS were stored at −70° C. and −30° C., respectively. Prior to use, the DS was thawed at room temperature for 72 hours. Aliquots of 150 mL were taken and mixed by inversion for 2 min. The DS was then filtered through a 0.22 μm polyvinylidene fluoride (PVDF) filter prior to filling in vials.

Table 2 and Table 3 cover equipment and vial components used for this study. 6R vials were depyrogenated prior to use. Sterilized stoppers were stored in autoclaved packaging prior to use, but without maintaining a sterile seal after the package was opened.

TABLE 2
Equipment used in experiments

| Equipment | Manufacturer |
|---|---|
| Millrock REVO Pro Lyo Unit | Millrock |
| Repeater pipette | Brandtech Scientific |
| Karl Fischer Moisture Analyzer | Metrohm |
| HIAC particle counter | Hach |
| HPLC | Agilent Technologies, |
| | Waters Instruments |
| pH Meter | Mettler Toledo |
| Osmometer | Advanced Instruments |
| Spectrophotometer | Agilent Technologies |

TABLE 3
Vial components
Component

Vial, 6R
Lyo Stopper, 20 mm
Seal, 20 mm

### Summary of Experiments

The purpose of these experiments was to compare the impact of a conventional lyophilization cycle with and without annealing with a CIN lyophilization cycle for the model high-concentration BCMA×CD3 bispecific molecule (20 mg/mL) and DLL3×CD3 bispecific molecule (15 mg/mL). A Bispecific molecule lyophilization cycle was used as the conventional lyophilization cycle with and without annealing. The CIN lyophilization cycle was based on past CIN development for the BCMA×CD3 bispecific molecule 1 mg 6R SKU⁷. A summary of all three lyophilization cycles can be found in Table 4.

TABLE 4
Lyophilization cycle summary

| Lyophilization | Utilizes | Utilizes | |
|---|---|---|---|
| cycle name | CIN? | Annealing? | Description |
| Non-CIN Standard | No | Yes | Bispecific molecule lyophilization cycle |
| Cycle | | | |
| Non-CIN Standard | No | No | A modification of the bispecific molecule lyophilization |
| Cycle without | | | cycle with the annealing step removed. Created only |
| Annealing | | | for the purposes of comparison. |
| CIN Cycle | Yes | No | A CIN cycle first developed for the BCMAxCD3 |
| | | | bispecific molecule 1 mg 6R SKU |

### Lyophilization Procedure

Prior to filling, 6R vials were used to completely fill a Millrock lyophilization tray. A total of 100 6R vials were filled with 1.3 mL of 20 mg/mL BCMA×CD3 bispecific molecule DS, and another 100 6R vials were filled with 1.3 mL of 15 mg/mL DLL3×CD3 bispecific molecule DS. The 1.3 mL fill in a 6R vial presentation was chosen as a high-concentration representation of a BiTE, consisting of a 1.28 mL fill of 5 mg/mL Bispecific molecule formulations in a 6R vial. The two sets of vials were arranged side-by-side in a single tray for lyophilization. Thermocouples were placed in the front, center, and back of the filled samples.

The lyophilization cycle parameters for the lyophilization cycle with and without annealing and the CIN cycle are given in Table 5. BCMA×CD3 bispecific molecule. The primary drying and secondary drying parameters remain unchanged. However, the nucleation conditions were adjusted to minimize the risk of HMWS formation. This involved reducing the nucleation temperature from less favourable −7° C. to more favourable −12, −15 or −17° C. according to the present invention, and set the post-nucleation to 90 min or even reduce the hold time from one hour to 30 or 20 minutes (FIG. 3). These changes created a CIN freezing phase that reduced the time the product spent in a cryo-concentrated “slushy” state, which reduces the chances of HMWS formation.

TABLE 5
Lyophilization cycle parameters

| | | | “Standard” | | | |
|---|---|---|---|---|---|---|
| | | | non-CIN | | | |
| | | “Standard” | cycle | CIN | CIN | CIN |
| Lyophilization | | non-CIN | without | cycle | cycle | cycle |
| Stage | Parameter | cycle | annealing | (center) | (low) | (high) |
| Freezing | CIN: Pre-nucleation | | | −15° C. | −17° C. | −12° C. |
| conditions | temperature (° C.) | | | and 110 | and 90 | and 120 |
| | and equilibration | | | min; −15° C. | min; −17° C. | min; −12° C. |
| | hold time (min); | | | and | and | and |
| | Post-nucleation | | | 30 min | 20 min | 90 min |
| | temperature (° C.) | | | | | |
| | and post-nucleation | | | | | |
| | hold time (h) | | | | | |
| | Freeze Ramp 1 | 0.5° C./min | | 0.2° C./min | 0.2° C./min | 0.2° C./min |
| | (° C./min) | | | | | |
| | Freeze | −45° C. and | | −45° C. | −50° C. | −40° C. |
| | temperature(° C.) | 2 h | | and 120 | and 150 | and 240 |
| | and hold time (h) | | | or 180 min | min | min |
| | Anneal ramp 1 | 0.5° C./min | | | | |
| | (° C./min) | | | | | |
| | Annealing | −12° C. and | 0.5° C./min | | | |
| | temperature (° C.) | 5 h | −45° C. and | | | |
| | and hold time (h) | 0.5° C./min | 2 h | | | |
| | Freeze Ramp 2 | −45° C. and | | | | |
| | (° C./min) | 2 h | | | | |
| | Freeze | | | | | |
| | temperature(° C.)/hold | | | | | |
| | time (h) | | | | | |
| Primary | PD Ramp (° C./min) | 0.3° C./min | 0.3° C./min | 0.3° C./min | 0.3° C./min | 0.3° C./min |
| Drying (PD) | PD temperature | −25° C. and | −25° C. and | −8° C. and | −15° C. and | −8° C. and |
| | (° C.) and minimum | 26 h | 27 h | 13 or | 13 or | 13 or |
| | hold time (h) | 70 mtorr | 70 mtorr | 50 h | 50 h | 50 h |
| | PD pressure (mtorr) | | | 100 mtorr | 75 mtorr | 100 mtorr |
| Secondary | SD Ramp (° C./min) | 0.1° C./min | 0.1° C./min | 0.1° C./min | 0.1° C./min | 0.1° C./min |
| Drying (SD) | SD temperature | 30° C. and | 30° C. and | 40° C. and | 37° C. and | 45° C. and |
| | (° C.) and hold | 8 h | 8 h | 8 h | 15 h | 8 h |
| | time (h) | 70 mtorr | 70 mtorr | 100 mtorr | 100 mtorr | 100 mtorr |
| | SD pressure (mtorr) | | | | | |

### Lyophilization Data Analysis

Lyophilization process data was exported from the instrument and analyzed for primary drying time and total cycle time, as well as product temperatures. Primary drying was considered to begin at the start of the primary drying temperature ramp. The endpoint of primary drying time was determined by observing when the instantaneous slope of the Pirani gauge approached zero. The total lyophilization time was the summation of the freezing, primary drying, and secondary drying stages of the lyophilization cycle.

The maximum product temperature of each vial containing a thermocouple was determined as the maximum observed temperature while the thermocouple remained within the frozen matrix during primary drying. This was done for each thermocouple to get a range of product temperatures across the lyophilization tray.

### Analytical Assays—BCMA×CD3 Bispecific Molecule and DLL3×CD3 Bispecific Molecule

BCMA×CD3 bispecific molecule and DLL3×CD3 bispecific molecule lyophilized samples were analyzed for product quality over 13 weeks. Samples were analyzed immediately following lyophilization for the initial timepoint (time zero). The remaining samples were then randomly sorted (with regard to lyophilization tray position) and placed at 4° C., 25° C. and 40% relative humidity (25° C./40RH), and 40° C. and 65% relative humidity (40° C./65RH). Samples were retrieved from the storage conditions for analytical testing at the specified timepoints (Table 5 and Table 7).

TABLE 5
BCMAxCD3 bispecific molecule Specification Information*

| | | Timepoints |
|---|---|---|
| Assay | Specification Limits | (wk) |
| Cation Exchange HPLC | Main peak: 69.8% to 89.8% | 0, 2, 4, 13 |
| (CEX-HPLC) | Acidic peaks: ≤30.2% | |
| | Basic peaks: ≤30.2% | |
| Size Exclusion | HMW: ≤10.0% | 0, 13 |
| Chromatography | | |
| (SE-UHPLC) | | |
| pH determination | 4.0 to 4.4 | 0 |
| Osmolality | 250 to 350 mOsm/kg | 0 |
| Subvisible particles HIAC | ≤6000/vial of 10 μm or | 0, 13 |
| quantitation | greater ≤600/vial of | |
| | 25 μm or greater | |
| Moisture content | ≤3.0% | 0, 13 |

BCMA×CD3 bispecific moleculeBCMA×CD3 bispecific moleculeDLL3×CD3 bispecific molecule

### Results and Discussion

### Lyophilization Results: Cycle Time and Maximum Observed Temperatures

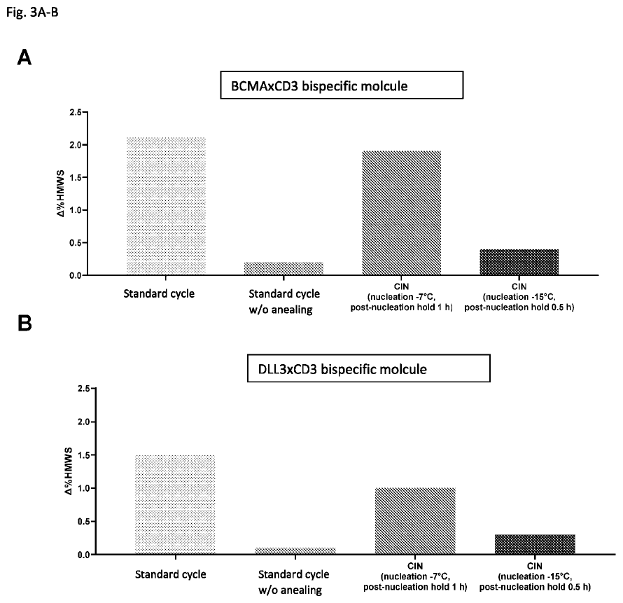

All lyophilization cycles were successfully completed. The chamber pressure (as measured by capacitance manometer and Pirani gauge), shelf temperature, and product temperatures of each lyophilization cycle are given in FIG. 2.

The non-CIN lyophilization cycle (FIG. 2A) had a maximum BCMA×CD3 bispecific molecule product temperature of −33.3° C. and a maximum DLL3×CD3 bispecific molecule product temperature of −33.1° C. during primary drying. The non-CIN lyophilization cycle without annealing (FIG. 2B) had 2° C. higher BCMA×CD3 bispecific molecule product temperatures (−31.3° C.) and 1.6° C. higher DLL3×CD3 bispecific molecule product temperatures (−31.5° C.) than the non-CIN lyophilization cycle during primary drying at the same shelf temperature setpoint. This is consistent with increased resistance to sublimation due to the removal of annealing. However, the non-CIN lyophilization cycle without annealing had a similar primary drying endpoint compared to the non-CIN lyophilization cycle with annealing. This indicates that annealing did not provide a time-saving benefit for these high-concentration bispecific molecules using the non-CIN lyophilization cycle.

The CIN cycle (FIG. 2C) with a primary drying shelf setpoint of −8° C. had a 50% reduction in primary drying time compared to the lyophilization cycle. CIN can produce faster primary drying times as CIN removes stochastic freezing altogether to form larger and more homogenous ice crystals that promote faster sublimation through a more porous cake structure². However, the observed maximum product temperatures for BCMA×CD3 bispecific molecule and DLL3×CD3 bispecific molecule (−27.8° C. and −28.7° C., respectively) were also higher than the previously reported critical temperature of −30° C. for BCMA×CD3 bispecific molecule at 1 mg/mL and DLL3×CD3 bispecific molecule at 5 mg/mL when using lyophilization with CIN.

A list of the minimum primary drying time and minimum total cycle time of the lyophilization cycles tested is given in Table 6. Since the primary drying time was determined when the instantaneous slope of the Pirani gauge approached zero, it is considered the minimum primary drying time without the added safety buffer that would be included in a usual lyophilization recipe. The total cycle time calculated using the minimum primary drying time is therefore considered the minimum total cycle time. The lyophilization non-CIN cycle with annealing has a longer minimum total cycle time than the standard non-CIN cycle without annealing. This is because the two cycles had similar primary drying and secondary drying times, but the annealing step added 7 hours to the freezing time of the standard cycle.

The CIN cycle had a primary drying endpoint of 13 hours, which is a 32% reduction in minimum total cycle time. While this may change based on optimization of the primary drying shelf temperature to reduce maximum product temperatures, CIN has the potential to produce faster primary drying times by forming larger and more homogenous ice crystals for a more porous cake structure. This, along with less time in the freezing phase, makes CIN the better option in terms of total cycle time.

TABLE 6
Lab-scale primary drying time and total cycle time

| | Lab-scale | | |
|---|---|---|---|
| | Minimum Primary | Minimum | % Total cycle time |
| | drying endpoint | total cycle | reduction compared to |
| Lyophilization Process | time (h) | time (h) | lyophilization cycle |
| Lyophilization (annealing) | 26 | 57 | — |
| non-CIN cycle | | | |
| Lyophilization non-CIN cycle | 27 | 48 | 16% |
| without annealing | | | |
| CIN cycle | 13 | 39 | 32% |

### HMWS Product Quality Results

Following lyophilization, samples were reconstituted and analyzed for % HMWS by SE-UHPLC, and the results were compared to the pre-lyophilized sample. The difference between the % HMWS of the reconstituted sample and the % HMWS of the sample prior to lyophilization was defined as Δ % HMWS. While all results were within the specification limit of ≤10.0% HMWS, the Δ % HMWS upon lyophilization was greatest in the samples from the standard cycle with annealing for both BCMA×CD3 bispecific molecule and DLL3×CD3 bispecific molecule. The samples from the standard cycle without annealing had the least HMWS growth upon lyophilization.

CIN-lyophilized samples had slightly more Δ % HMWS than those of the standard cycle without annealing, but still had approximately 80% less Δ % HMWS than samples from the standard cycle with annealing for both BCMA×CD3 bispecific molecule and DLL3×CD3 bispecific molecule. As CIN samples spend less time in a partially frozen matrix, there is less risk of aggregation and HMWS formation in CIN lyophilization cycles than in annealing cycles.

After storage of lyophilized samples at 4° C., 25° C./40RH, and 40° C./65RH, samples were removed at specified timepoints for % HMWS analysis. DLL3×CD3 bispecific molecule samples were measured at time zero, 2 weeks, and 4 weeks, and BCMA×CD3 bispecific molecule samples were measured at time zero and 13 weeks. While BCMA×CD3 bispecific molecule samples were originally planned to have 2 and 4 week timepoints as well, instrument issues caused a 13 week timepoint to be measured instead.

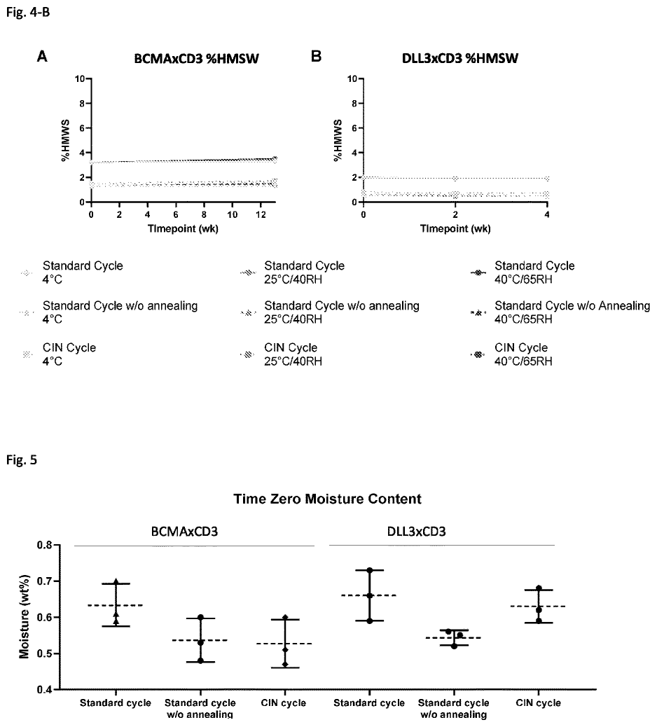

None of the lyophilization groups showed an increase in % HMWS over time (Δ % HMWS was not used in the case of comparing stability timepoints). All BCMA×CD3 bispecific molecule samples (FIG. 4A) and DLL3×CD3 bispecific molecule samples (FIG. 4B) maintained % HMWS over time. The lyophilization cycle with annealing consistently had higher % HMWS than the lyophilization cycle without annealing and the CIN cycle.

### BCMA×CD3 Bispecific Molecule and DLL3×CD3 Bispecific Molecule Moisture Content

All moisture results were ≤3.0%. Three samples were measured at each timepoint, and samples from the front, middle, and back of the lyophilization shelf were measured at time zero, and no significant difference in moisture content was found between the samples from the three lyophilization conditions (p>0.05), as shown in FIG. 5.

The CIN cycle in this example used the same primary drying temperature setpoint as the CIN cycle for the BCMA×CD3 bispecific molecule 1 mg 6R This caused a higher product temperature during primary drying of high-concentration bispecific molecules in this study than was observed in the previous study for 1 mg/mL product due to higher cake resistance in the high-concentration formulation. As discussed above, the product temperature of CIN samples exceeded the previously characterized critical temperature of 30° C. for CIN-lyophilized bispecific molecules. It was observed in a previous study using 1 mg/mL and 5 mg/mL BiTE standard products that exceeding this temperature during primary drying caused increasing moisture content over time, which was also observed in this example. Optimization of the CIN lyophilization cycle primary drying temperature would likely mitigate this issue.

### BCMA×CD3 Bispecific Molecule and DLL3×CD3 Bispecific Molecule CEX Results

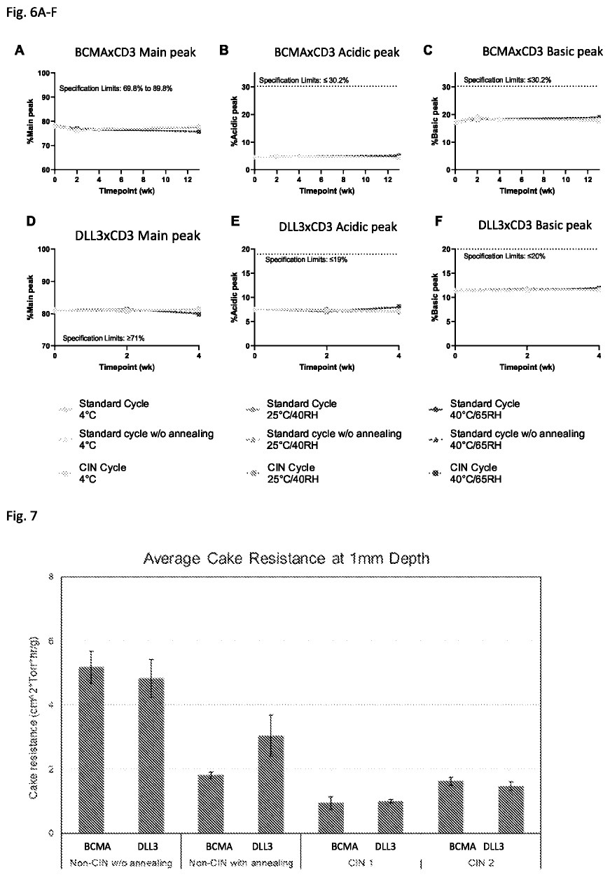

BCMA×CD3 bispecific molecule and DLL3×CD3 bispecific molecule CEX-HPLC assay results showed minimal differences between samples from each lyophilization cycle. A slight decrease in main peak area and a corresponding slight increase in acidic and basic peak area can be seen in BCMA×CD3 bispecific molecule samples stored at 40° C./65RH at 13 weeks, but this was observed regardless of lyophilization cycle (FIG. 6A-C). The same behavior was observed for DLL3×CD3 bispecific molecule samples at 4 weeks (FIG. 6D-F). Thus, there was no observed difference between the lyophilization cycle with and without annealing and the CIN cycle.

### BCMA×CD3 Bispecific Molecule and DLL3×CD3 Bispecific Molecule HIAC Particle Quantitation Results

Particle quantitation of BCMA×CD3 bispecific molecule (Table 7) and DLL3×CD3 bispecific molecule (Table 8) showed similar amounts of particles between samples from the lyophilization cycle with and without annealing and samples from the CIN cycle. All particle counts were well below the specification limit of 6000 particles/vial ≥10 μm and 600 particles/vial ≥25 μm.

TABLE 7
BCMAxCD3 bispecific molecule HIAC particle quantitation

| | | | | Standard Cycle | | | | | |
|---|---|---|---|---|---|---|---|---|---|
| | Standard Cycle | | | w/o annealing | | | CIN Cycle | | |
| | (particles/vial) | | | (particles/vial) | | | (particles/vial) | | |
| ≥10 μm | | 25° | 40° | | 25° | 40° | | 25° | 40° |
| Timepoint (wk) | 4° C. | C./40RH | C./65RH | 4° C. | C./40RH | C./65RH | 4° C. | C./40RH | C./65RH |
| 0 | 64 | — | — | 20 | — | — | 16 | — | — |
| 13 | 3 | 7 | 6 | 38 | 25 | 13 | 6 | 6 | 13 |
| | | | | Standard Cycle | | | | | |
| | Standard Cycle | | | w/o annealing | | | CIN Cycle | | |
| | (particles/vial) | | | (particles/vial) | | | (particles/vial) | | |
| ≥25 μm | | 25° | 40° | | 25° | 40° | | 25° | 40° |
| Timepoint (wk) | 4° C. | C./40RH | C./65RH | 4° C. | C./40RH | C./65RH | 4° C. | C./40RH | C./65RH |
| 0 | 3 | — | — | 6 | — | — | 0 | — | — |
| 13 | 0 | 0 | 0 | 0 | 0 | 6 | 0 | 0 | 0 |

TABLE 8
DLL3xCD3 bispecific molecule HIAC particle quantitation

| | | | | Standard Cycle | | | | | |
|---|---|---|---|---|---|---|---|---|---|
| | Standard Cycle | | | w/o annealing | | | CIN Cycle | | |
| | (particles/vial) | | | (particles/vial) | | | (particles/vial) | | |
| ≥10 μm | | 25° C./ | 40° C./ | | 25° C./ | 40° C./ | | 25° C./ | 40° C./ |
| Timepoint (wk) | 4° C. | 40RH | 65RH | 4° C. | 40RH | 65RH | 4° C. | 40RH | 65RH |
| 0 | 13 | — | — | 19 | — | — | 13 | — | — |
| 13 | 12 | 7 | 3 | 16 | 13 | 7 | 25 | 12 | 12 |
| | | | | Standard Cycle | | | | | |
| | Standard Cycle | | | w/o annealing | | | CIN Cycle | | |
| | (particles/vial) | | | (particles/vial) | | | (particles/vial) | | |
| ≥25 μm | | 25° C./ | 40° C./ | | 25° C./ | 40° C./ | | 25° C./ | 40° C./ |
| Timepoint (wk) | 4° C. | 40RH | 65RH | 4° C. | 40RH | 65RH | 4° C. | 40RH | 65RH |
| 0 | 3 | — | — | 0 | — | — | 0 | — | — |
| 13 | 0 | 0 | 0 | 0 | 0 | 0 | 0 | 0 | 0 |

### BCMA×CD3 Bispecific Molecule and DLL3×CD3 Bispecific Molecule pH and Osmolality Results

At time zero, pH and osmolality measurements were taken of the reconstituted samples from the standard cycle with and without annealing and the CIN cycle. Osmolality and pH were tested to confirm product formulation and reconstitution. No differences in pH or osmolality were observed between pre-lyophilization samples and post-lyophilization reconstituted samples for both BCMA×CD3 bispecific molecule (Table 9) and DLL3×CD3 bispecific molecule (Table 10). Due to this, pH and osmolality were not monitored for any other timepoints during the stability testing.

TABLE 9
BCMAxCD3 bispecific molecule pH and osmolality measurements
Before lyophilization

| | Pre-lyophilization | Pre-lyophilization |
|---|---|---|
| | pH | osmolality |
| BCMAxCD3 bispecific | 4.28 | 319 |
| molecule DS | | |

After lyophilization

| Cycle name | Time zero pH | Time zero osmolality |
|---|---|---|
| Standard Cycle | 4.26 | 317 |
| Standard Cycle w/o annealing | 4.25 | 315 |
| CIN Cycle | 4.26 | 318 |

TABLE 10
DLL3xCD3 bispecific molecule pH and osmolality measurements
Before lyophilization

| | Pre-lyophilization | Pre-lyophilization |
|---|---|---|
| | pH | Osmolality |
| DLL3xCD3 bispecific | 4.37 | 333 |
| molecule DS | | |

After lyophilization

| Cycle name | Time zero pH | Time zero Osmolality |
|---|---|---|
| Standard Cycle | 4.35 | 333 |
| Standard Cycle w/o annealing | 4.30 | 331 |
| CIN Cycle | 4.30 | 333 |

### Conclusions

Controlled ice nucleation lyophilization was evaluated in use with high-concentration products, using BCMA×CD3 bispecific molecule (20 mg/mL) and DLL3×CD3 bispecific molecule (15 mg/mL) in a 1.3 mL fill in 6R vial presentation as model proteins. CIN lyophilization was able to avoid the high levels of HMWS commonly observed when using a lyophilization non-CIN cycle with annealing for these products. CIN-lyophilized samples also had less HMWS than the annealed-lyophilized samples over 13 weeks of product quality testing, regardless of storage condition.

CIN lyophilization also reduced the minimum total cycle time by 32% compared to the bispecific molecule lyophilization non-CIN cycle. Optimization of primary drying parameters lead to less reduction in minimum total cycle time. However, CIN lyophilization primarily provides the benefit of reduced HMWS formation in high-concentration bispecific molecules with the potential benefit of reduced lyophilization cycle time, making it an attractive option compared to a lyophilization non-CIN cycle with annealing.

All of the references cited herein, including patents, patent applications, literature publications, and the like, are hereby incorporated in their entireties by reference.

While this invention has been described with an emphasis upon preferred embodiments, it will be obvious to those of ordinary skill in the art that variations of the preferred compounds and methods may be used and that it is intended that the invention may be practiced otherwise than as specifically described herein. Accordingly, this invention includes all modifications encompassed within the spirit and scope of the invention as defined by the following claims.

TABLE 13
Sequence table

| | | | | |
|---|---|---|---|---|
| 1. | CD19 VL CDR1 | artificial | aa | KASQSVDYDGDSYLN |
| 2. | CD19 VL CDR2 | artificial | aa | DASNLVS |
| 3. | CD19 VL CDR3 | artificial | aa | QQSTEDPWT |
| 4. | CD19 VH CDR1 | artificial | aa | SYWMN |
| 5. | CD19 VH CDR2 | artificial | aa | QIWPGDGDTNYNGKFKG |
| 6. | CD19 VH CDR3 | artificial | aa | RETTTVGRYYYAMDY |
| 7. | CD19 VL | artificial | aa | DIQLTQSPASLAVSLGQRATISCKASQSVDYDGDSYLNW |
| | | | | YQQIPGQPPKLLIYDASNLVSGIPPRFSGSGSGTDFTLNI |
| | | | | HPVEKVDAATYHCQQSTEDPWTFGGGTKLEIK |
| 8. | CD19 VH | artificial | aa | QVQLQQSGAELVRPGSSVKISCKASGYAFSSYWMNWV |
| | | | | KQRPGQGLEWIGQIWPGDGDTNYNGKFKGKATLTADES |
| | | | | SSTAYMQLSSLASEDSAVYFCARRETTTVGRYYYAMDY |
| | | | | WGQGTTVTVSS |
| 9. | CD3 VH CDR1 | artificial | aa | RYTMH |
| 10. | CD3 VH CDR2 | artificial | aa | YINPSRGYTNYNQKFKD |
| 11. | CD3 VH CDR3 | artificial | aa | YYDDHYCLDY |
| 12. | CD3 VL CDR1 | artificial | aa | RASSSVSYMN |
| 13. | CD3 VL CDR2 | artificial | aa | DTSKVAS |
| 14. | CD3 VL CDR3 | artificial | aa | QQWSSNPLT |
| 15. | CD3 VH | artificial | aa | DIKLQQSGAELARPGASVKMSCKTSGYTFTRYTMHWVK |
| | | | | QRPGQGL |
| | | | | EWIGYINPSRGYTNYNQKFKDKATLTTDKSSSTAYMQLS |
| | | | | SLTSEDSAVYYCARYYDDHYCLDYWGQGTTLTVSS |
| 16. | CD3 VL | artificial | aa | VDDIQLTQSPAIMSASPGEKVTMTCRASSSVSYMNWYQ |
| | | | | QKSGTSPKRWIYDTSKVASGVPYRFSGSGSGTSYSLTIS |
| | | | | SMEAEDAATYYCQQWSSNPLTFGAGTKLELK |
| 17. | CD19xCD3 scFv | artificial | aa | DIQLTQSPASLAVSLGQRATISCKASQSVDYDGDSYLNW |
| | incl linker and | | | YQQIPGQPPKLLIYDASNLVSGIPPRFSGSGSGTDFTLNI |
| | his-tag | | | HPVEKVDAATYHCQQSTEDPWTFGGGTKLEIKGGGGS |
| | | | | GGGGSGGGGSQVQLQQSGAELVRPGSSVKISCKASGY |
| | | | | AFSSYWMNWVKQRPGQGLEWIGQIWPGDGDTNYNGK |
| | | | | FKGKATLTADESSSTAYMQLSSLASEDSAVYFCARRETT |
| | | | | TVGRYYYAMDYWGQGTTVTVSSGGGGSDIKLQQSGAE |
| | | | | LARPGASVKMSCKTSGYTFTRYTMHWVKQRPGQGLEW |
| | | | | IGYINPSRGYTNYNQKFKDKATLTTDKSSSTAYMQLSSLT |
| | | | | SEDSAVYYCARYYDDHYCLDYWGQGTTLTVSSVEGGS |
| | | | | GGSGGSGGSGGVDDIQLTQSPAIMSASPGEKVTMTCRA |
| | | | | SSSVSYMNWYQQKSGTSPKRWIYDTSKVASGVPYRFS |
| | | | | GSGSGTSYSLTISSMEAEDAATYYCQQWSSNPLTFGAG |
| | | | | TKLELKHHHHHH |
| 18. | CDR-L1 of I2C | artificial | aa | GSSTGAVTSGNYPN |
| 19. | CDR-L2 of I2C | artificial | aa | GTKFLAP |
| 20. | CDR-L3 of I2C | artificial | aa | VLWYSNRWV |
| 21. | CDR-H1 of I2C | artificial | aa | KYAMN |
| 22. | CDR-H2 of I2C | artificial | aa | RIRSKYNNYATYYADSVKD |
| 23. | CDR-H3 of I2C | artificial | aa | HGNFGNSYISYWAY |
| 24. | VH of I2C | artificial | aa | EVQLVESGGGLVQPGGSLKLSCAASGFTFNKYAMNW |
| | | | | VRQAPGKGLEWVARIRSKYNNYATYYADSVKDRFTISR |
| | | | | DDSKNTAYLQMNNLKTEDTAVYYCVRHGNFGNSYISY |
| | | | | WAYWGQGTLVTVSS |
| 25. | VL of I2C | artificial | aa | QTVVTQEPSLTVSPGGTVTLTCGSSTGAVTSGNYPNW |
| | | | | VQQKPGQAPRGLIGGTKFLAPGTPARFSGSLLGGKAAL |
| | | | | TLSGVQPEDEAEYYCVLWYSNRWVFGGGTKLTVL |
| 26. | VH-VL of I2C | artificial | aa | EVQLVESGGGLVQPGGSLKLSCAASGFTFNKYAMNW |
| | | | | VRQAPGKGLEWVARIRSKYNNYATYYADSVKDRFTISR |
| | | | | DDSKNTAYLQMNNLKTEDTAVYYCVRHGNFGNSYISY |
| | | | | WAYWGQGTLVTVSSGGGGSGGGGSGGGGSQTWTQ |
| | | | | EPSLTVSPGGTVTLTCGSSTGAVTSGNYPNWVQQKPG |
| | | | | QAPRGLIGGTKFLAPGTPARFSGSLLGGKAALTLSGVQ |
| | | | | PEDEAEYYCVLWYSNRWVFGGGTKLTVL |
| 27. | CD33 ccVH of | artificial | aa | QVQLVQSGAEVKKPGESVKVSCKASGYTFTNYGMNW |
| | E11 | | | VKQAPGQCLEWMGWINTYTGEPTYADKFQGRVTMTT |
| | | | | DTSTSTAYMEIRNLGGDDTAVYYCARWSWSDGYYVYF |
| | | | | DYWGQGTSVTVSS |
| 28. | CD33 VH of E11 | Artificial | aa | QVQLVQSGAEVKKPGESVKVSCKASGYTFTNYGMNW |
| | | | | VKQAPGQGLEWMGWINTYTGEPTYADKFQGRVTMTT |
| | | | | DTSTSTAYMEIRNLGGDDTAVYYCARWSWSDGYYVYF |
| | | | | DYWGQGTSVTVSS |
| 29. | CD33 HCDR1 of | artificial | aa | NYGMN |
| | E11 | | | |
| 30. | CD33 HCDR2 of | artificial | aa | WINTYTGEPTYADKFQG |
| | E11 | | | |
| 31. | CD33 HCDR3 of | artificial | aa | WSWSDGYYVYFDY |
| | E11 | | | |
| 32. | CD33 CC VL of | artificial | aa | DIVMTQSPDSLTVSLGERTTINCKSSQSVLDSSTNKNSL |
| | E11 | | | AWYQQKPGQPPKLLLSWASTRESGIPDRFSGSGSGTD |
| | | | | FTLTIDSPQPEDSATYYCQQSAHFPITFGCGTRLEIK |
| 33. | CD33 VL of E11 | artificial | aa | DIVMTQSPDSLTVSLGERTTINCKSSQSVLDSSTNKNSL |
| | | | | AWYQQKPGQPPKLLLSWASTRESGIPDRFSGSGSGTD |
| | | | | FTLTIDSPQPEDSATYYCQQSAHFPITFGQGTRLEIK |
| 34. | CD33 LCDR1 of | artificial | aa | KSSQSVLDSSTNKNSLA |
| | E11 | | | |
| 35. | CD33 LCDR2 of | artificial | aa | WASTRES |
| | E11 | | | |
| 36. | CD33 LCDR3 of | artificial | aa | QQSAHFPIT |
| | E11 | | | |
| 37. | CD33 HL CC of | artificia | aa | QVQLVQSGAEVKKPGESVKVSCKASGYTFTNYGMNW |
| | E11 | | | VKQAPGQCLEWMGWINTYTGEPTYADKFQGRVTMTT |
| | | | | DTSTSTAYMEIRNLGGDDTAVYYCARWSWSDGYYVYF |
| | | | | DYWGQGTSVTVSSGGGGSGGGGSGGGGSDIVMTQS |
| | | | | PDSLTVSLGERTTINCKSSQSVLDSSTNKNSLAWYQQK |
| | | | | PGQPPKLLLSWASTRESGIPDRFSGSGSGTDFTLTIDS |
| | | | | PQPEDSATYYCQQSAHFPITFGCGTRLEIK |
| 38. | CD33 HL of E11 | artificial | aa | QVQLVQSGAEVKKPGESVKVSCKASGYTFTNYGMNW |
| | | | | VKQAPGQGLEWMGWINTYTGEPTYADKFQGRVTMTT |
| | | | | DTSTSTAYMEIRNLGGDDTAVYYCARWSWSDGYYVYF |
| | | | | DYWGQGTSVTVSSGGGGSGGGGSGGGGSDIVMTQS |
| | | | | PDSLTVSLGERTTINCKSSQSVLDSSTNKNSLAWYQQK |
| | | | | PGQPPKLLLSWASTRESGIPDRFSGSGSGTDFTLTIDS |
| | | | | PQPEDSATYYCQQSAHFPITFGQGTRLEIK |
| 39. | CD33 CC E11 HL | artificial | aa | QVQLVQSGAEVKKPGESVKVSCKASGYTFTNYGMNW |
| | x I2C HL | | | VKQAPGQCLEWMGWINTYTGEPTYADKFQGRVTMTT |
| | Bispecific | | | DTSTSTAYMEIRNLGGDDTAVYYCARWSWSDGYYVYF |
| | molecule | | | DYWGQGTSVTVSSGGGGSGGGGSGGGGSDIVMTQS |
| | | | | PDSLTVSLGERTTINCKSSQSVLDSSTNKNSLAWYQQK |
| | | | | PGQPPKLLLSWASTRESGIPDRFSGSGSGTDFTLTIDS |
| | | | | PQPEDSATYYCQQSAHFPITFGCGTRLEIKSGGGGSEV |
| | | | | QLVESGGGLVQPGGSLKLSCAASGFTFNKYAMNWVR |
| | | | | QAPGKGLEWVARIRSKYNNYATYYADSVKDRFTISRDD |
| | | | | SKNTAYLQMNNLKTEDTAVYYCVRHGNFGNSYISYWA |
| | | | | YWGQGTLVTVSSGGGGSGGGGSGGGGSQTWTQEP |
| | | | | SLTVSPGGTVTLTCGSSTGAVTSGNYPNWVQQKPGQ |
| | | | | APRGLIGGTKFLAPGTPARFSGSLLGGKAALTLSGVQP |
| | | | | EDEAEYYCVLWYSNRWVFGGGTKLTVL |
| 40. | CD33 E11 HL x | artificial | aa | MGWSCIILFLVATATGVHSQVQLVQSGAEVKKPGESVK |
| | I2C HL | | | VSCKASGYTFTNYGMNWVKQAPGQGLEWMGWINTYT |
| | | | | GEPTYADKFQGRVTMTTDTSTSTAYMEIRNLGGDDTA |
| | | | | VYYCARWSWSDGYYVYFDYWGQGTSVTVSSGGGGS |
| | | | | GGGGSGGGGSDIVMTQSPDSLTVSLGERTTINCKSSQ |
| | | | | SVLDSSTNKNSLAWYQQKPGQPPKLLLSWASTRESGI |
| | | | | PDRFSGSGSGTDFTLTIDSPQPEDSATYYCQQSAHFP\| |
| | | | | TFGQGTRLEIKSGGGGSEVQLVESGGGLVQPGGSLKL |
| | | | | SCAASGFTFNKYAMNWVRQAPGKGLEWVARIRSKYN |
| | | | | NYATYYADSVKDRFTISRDDSKNTAYLQMNNLKTEDTA |
| | | | | VYYCVRHGNFGNSYISYWAYWGQGTLVTVSSGGGGS |
| | | | | GGGGSGGGGSQTVVTQEPSLTVSPGGTVTLTCGSST |
| | | | | GAVTSGNYPNWVQQKPGQAPRGLIGGTKFLAPGTPAR |
| | | | | FSGSLLGGKAALTLSGVQPEDEAEYYCVLWYSNRWVF |
| | | | | GGGTKLTVLHHHHHH |
| 41. | CD33 CC x I2C- | artificial | aa | QVQLVQSGAEVKKPGESVKVSCKASGYTFTNYGMNWV |
| | scFc Bispecific | | | KQAPGQCLEWMGWINTYTGEPTYADKFQGRVTMTTDT |
| | HLE molecule | | | STSTAYMEIRNLGGDDTAVYYCARWSWSDGYYVYFDY |
| | | | | WGQGTSVTVSSGGGGSGGGGSGGGGSDIVMTQSPDS |
| | | | | LTVSLGERTTINCKSSQSVLDSSTNKNSLAWYQQKPGQ |
| | | | | PPKLLLSWASTRESGIPDRFSGSGSGTDFTLTIDSPQPE |
| | | | | DSATYYCQQSAHFPITFGCGTRLEIKSGGGGSEVQLVES |
| | | | | GGGLVQPGGSLKLSCAASGFTFNKYAMNWVRQAPGKG |
| | | | | LEWVARIRSKYNNYATYYADSVKDRFTISRDDSKNTAYL |
| | | | | QMNNLKTEDTAVYYCVRHGNFGNSYISYWAYWGQGTL |
| | | | | VTVSSGGGGSGGGGSGGGGSQTVVTQEPSLTVSPGGT |
| | | | | VTLTCGSSTGAVTSGNYPNWVQQKPGQAPRGLIGGTKF |
| | | | | LAPGTPARFSGSLLGGKAALTLSGVQPEDEAEYYCVLW |
| | | | | YSNRWVFGGGTKLTVLGGGGDKTHTCPPCPAPELLGG |
| | | | | PSVFLFPPKPKDTLMISRTPEVTCVVVDVSHEDPEVKFN |
| | | | | WYVDGVEVHNAKTKPCEEQYGSTYRCVSVLTVLHQDW |
| | | | | LNGKEYKCKVSNKALPAPIEKTISKAKGQPREPQVYTLPP |
| | | | | SREEMTKNQVSLTCLVKGFYPSDIAVEWESNGQPENNY |
| | | | | KTTPPVLDSDGSFFLYSKLTVDKSRWQQGNVFSCSVMH |
| | | | | EALHNHYTQKSLSLSPGKGGGGSGGGGSGGGGSGGG |
| | | | | GSGGGGSGGGGSDKTHTCPPCPAPELLGGPSVFLFPP |
| | | | | KPKDTLMISRTPEVTCVVVDVSHEDPEVKFNWYVDGVE |
| | | | | VHNAKTKPCEEQYGSTYRCVSVLTVLHQDWLNGKEYKC |
| | | | | KVSNKALPAPIEKTISKAKGQPREPQVYTLPPSREEMTK |
| | | | | NQVSLTCLVKGFYPSDIAVEWESNGQPENNYKTTPPVLD |
| | | | | SDGSFFLYSKLTVDKSRWQQGNVFSCSVMHEALHNHYT |
| | | | | QKSLSLSPGK |
| 42. | EGFRvIIIxCD3- | artificial | aa | nygmh |
| | scFc VH CDR1 | | | |
| 43. | EGFRvIIIxCD3- | artificial | aa | viwydgsdkyyadsvrg |
| | scFc VH CDR2 | | | |
| 44. | EGFRvIIIxCD3- | artificial | aa | dgydiltgnprdfdy |
| | scFc VH CDR3 | | | |
| 45. | EGFRvIIIxCD3- | artificial | aa | rssqslvhsdgntyls |
| | scFc VL CDR1 | | | |
| 46. | EGFRvIIIxCD3- | artificial | aa | risrrfs |
| | scFc VL CDR2 | | | |
| 47. | EGFRvIIIxCD3- | artificial | aa | mqsthvprt |
| | scFc VL CDR3 | | | |
| 48. | EGFRvIII_CCxCD3- | artificial | aa | qvqlvesgggvvqsgrslrlscaasgftfrnygmhwvrqapgkclewvaviwy |
| | scFc VH | | | dgsdkyyadsvrgrftisrdnskntlylqmnslraedtavyycardgydiltgnpr |
| | | | | dfdywgqgtlvtvss |
| 49. | EGFRvIII_CCxCD3- | artificial | aa | dtvmtqtplsshvtlgqpasiscrssqslvhsdgntylswlqqrpgqpprlliyrisr |
| | scFc VL | | | rfsgvpdrfsgsgagtdftleisrveaedvgvyycmqsthvprtfgcgtkveik |
| 50. | EGFRvIII_CCxCD3- | artificial | aa | qvqlvesgggvvqsgrslrlscaasgftfrnygmhwvrqapgkclewvaviwy |
| | scFc scFv | | | dgsdkyyadsvrgrftisrdnskntlylqmnslraedtavyycardgydiltgnpr |
| | | | | dfdywgqgtlvtvssggggsggggsggggsdtvmtqtplsshvtlgqpasiscr |
| | | | | ssqslvhsdgntylswlqqrpgqpprlliyrisrrfsgvpdrfsgsgagtdftleisrv |
| | | | | eaedvgvyycmqsthvprtfgcgtkveik |
| 51. | EGFRvIII_CCxCD3- | artificial | aa | qvqlvesgggvvqsgrslrlscaasgftfrnygmhwvrqapgkclewvaviwy |
| | scFc Bispecific | | | dgsdkyyadsvrgrftisrdnskntlylqmnslraedtavyycardgydiltgnpr |
| | molecule | | | dfdywgqgtlvtvssggggsggggsggggsdtvmtqtplsshvtlgqpasiscr |
| | | | | ssqslvhsdgntylswlqqrpgqpprlliyrisrrfsgvpdrfsgsgagtdftleisrv |
| | | | | eaedvgvyycmqsthvprtfgcgtkveiksggggsevqlvesggglvqpggsl |
| | | | | klscaasgftfnkyamnwvrqapgkglewvarirskynnyatyyadsvkdrfti |
| | | | | srddskntaylqmnnlktedtavyycvrhgnfgnsyisywaywgqgtlvtvssg |
| | | | | gggsggggsggggsqtvvtqepsltvspggtvtltcgsstgavtsgnypnwvq |
| | | | | qkpgqaprgliggtkflapgtparfsgsllggkaaltlsgvqpedeaeyycvlwys |
| | | | | nrwvfgggtkltvl |
| 52. | EGFRvIII_CCxCD3- | artificial | aa | qvqlvesgggvvqsgrslrlscaasgftfrnygmhwvrqapgkclewvaviwy |
| | scFc Bispecific | | | dgsdkyyadsvrgrftisrdnskntlylqmnslraedtavyycardgydiltgnpr |
| | HLE molecule | | | dfdywgqgtlvtvssggggsggggsggggsdtvmtqtplsshvtlgqpasiscr |
| | | | | ssqslvhsdgntylswlqqrpgqpprlliyrisrrfsgvpdrfsgsgagtdftleisrv |
| | | | | eaedvgvyycmqsthvprtfgcgtkveiksggggsevqlvesggglvqpggsl |
| | | | | klscaasgftfnkyamnwvrqapgkglewvarirskynnyatyyadsvkdrfti |
| | | | | srddskntaylqmnnlktedtavyycvrhgnfgnsyisywaywgqgtlvtvssg |
| | | | | gggsggggsggggsqtvvtqepsltvspggtvtltogsstgavtsgnypnwvq |
| | | | | qkpgqaprgliggtkflapgtparfsgsllggkaaltlsgvqpedeaeyycvlwys |
| | | | | nrwvfgggtkltvlggggdkthtcppcpapellggpsvflfppkpkdtlmisrtpev |
| | | | | tcvvvdvshedpevkfnwyvdgvevhnaktkpceeqygstyrcvsvltvlhqd |
| | | | | wlngkeykckvsnkalpapiektiskakgqprepqvytlppsreemtknqvslt |
| | | | | clvkgfypsdiavewesngqpennykttppvldsdgsfflyskltvdksrwqqg |
| | | | | nvfscsvmhealhnhytqkslslspgkggggsggggsggggsggggsggg |
| | | | | gsggggsdkthtcppcpapellggpsvflfppkpkdtlmisrtpevtcvvvdvsh |
| | | | | edpevkfnwyvdgvevhnaktkpceeqygstyrcvsvltvlhqdwlngkeyk |
| | | | | ckvsnkalpapiektiskakgqprepqvytlppsreemtknqvsltclvkgfyps |
| | | | | diavewesngqpennykttppvldsdgsfflyskltvdksrwqqgnvfscsvm |
| | | | | healhnhytqkslslspgk |
| 53. | MSLN_5 VH | artificial | aa | DYYMT |
| | CDR1 | | | |
| 54. | MSLN_5 VH | artificial | aa | YISSSGSTIYYADSVKG |
| | CDR2 | | | |
| 55. | MSLN_5 VH | artificial | aa | DRNSHFDY |
| | CDR3 | | | |
| 56. | MSLN_5 VL | artificial | aa | RASQGINTWLA |
| | CDR1 | | | |
| 57. | MSLN_5 VL | artificial | aa | GASGLQS |
| | CDR2 | | | |
| 58. | MSLN_5 VL | artificial | aa | QQAKSFPRT |
| | CDR3 | | | |
| 59. | MSLN _5 VH | artificial | aa | QVQLVESGGGLVKPGGSLRLSCAASGFTFSDYYMTWI |
| | | | | RQAPGKGLEWLSYISSSGSTIYYADSVKGRFTISRDNA |
| | | | | KNSLFLQMNSLRAEDTAVYYCARDRNSHFDYWGQGT |
| | | | | LVTVSS |
| 60. | MSLN_5 VL | artificial | aa | DIQMTQSPSSVSASVGDRVTITCRASQGINTWLAWYQ |
| | | | | QKPGKAPKLLIYGASGLQSGVPSRFSGSGSGTDFTLTI |
| | | | | SSLQPEDFATYYCQQAKSFPRTFGQGTKVEIK |
| 61. | MSLN_5 scFv | artificial | aa | QVQLVESGGGLVKPGGSLRLSCAASGFTFSDYYMTWI |
| | | | | RQAPGKGLEWLSYISSSGSTIYYADSVKGRFTISRDNA |
| | | | | KNSLFLQMNSLRAEDTAVYYCARDRNSHFDYWGQGT |
| | | | | LVTVSSGGGGSGGGGSGGGGSDIQMTQSPSSVSASV |
| | | | | GDRVTITCRASQGINTWLAWYQQKPGKAPKLLIYGASG |
| | | | | LQSGVPSRFSGSGSGTDFTLTISSLQPEDFATYYCQQA |
| | | | | KSFPRTFGQGTKVEIK |
| 62. | MSLN_5xI2C0 | artificial | aa | QVQLVESGGGLVKPGGSLRLSCAASGFTFSDYYMTWI |
| | bispecific | | | RQAPGKGLEWLSYISSSGSTIYYADSVKGRFTISRDNA |
| | molecule | | | KNSLFLQMNSLRAEDTAVYYCARDRNSHFDYWGQGT |
| | | | | LVTVSSGGGGSGGGGSGGGGSDIQMTQSPSSVSASV |
| | | | | GDRVTITCRASQGINTWLAWYQQKPGKAPKLLIYGASG |
| | | | | LQSGVPSRFSGSGSGTDFTLTISSLQPEDFATYYCQQA |
| | | | | KSFPRTFGQGTKVEIKSGGGGSEVQLVESGGGLVQPG |
| | | | | GSLKLSCAASGFTFNKYAMNWVRQAPGKGLEWVARIR |
| | | | | SKYNNYATYYADSVKDRFTISRDDSKNTAYLQMNNLKT |
| | | | | EDTAVYYCVRHGNFGNSYISYWAYWGQGTLVTVSSG |
| | | | | GGGSGGGGSGGGGSQTVVTQEPSLTVSPGGTVTLTC |
| | | | | GSSTGAVTSGNYPNWVQQKPGQAPRGLIGGTKFLAP |
| | | | | GTPARFSGSLLGGKAALTLSGVQPEDEAEYYCVLWYS |
| | | | | NRWVFGGGTKLTVL |
| 63. | MSLN_5xCD3- | artificial | aa | QVQLVESGGGLVKPGGSLRLSCAASGFTFSDYYMTWI |
| | scFc Bispecific | | | RQAPGKGLEWLSYISSSGSTIYYADSVKGRFTISRDNA |
| | HLE molecule | | | KNSLFLQMNSLRAEDTAVYYCARDRNSHFDYWGQGTL |
| | | | | VTVSSGGGGSGGGGSGGGGSDIQMTQSPSSVSASVG |
| | | | | DRVTITCRASQGINTWLAWYQQKPGKAPKLLIYGASGL |
| | | | | QSGVPSRFSGSGSGTDFTLTISSLQPEDFATYYCQQAK |
| | | | | SFPRTFGQGTKVEIKSGGGGSEVQLVESGGGLVQPGG |
| | | | | SLKLSCAASGFTFNKYAMNWVRQAPGKGLEWVARIRS |
| | | | | KYNNYATYYADSVKDRFTISRDDSKNTAYLQMNNLKTE |
| | | | | DTAVYYCVRHGNFGNSYISYWAYWGQGTLVTVSSGG |
| | | | | GGSGGGGSGGGGSQTVVTQEPSLTVSPGGTVTLTCG |
| | | | | SSTGAVTSGNYPNWVQQKPGQAPRGLIGGTKFLAPGT |
| | | | | PARFSGSLLGGKAALTLSGVQPEDEAEYYCVLWYSNR |
| | | | | WVFGGGTKLTVLGGGGDKTHTCPPCPAPELLGGPSVF |
| | | | | LFPPKPKDTLMISRTPEVTCVVVDVSHEDPEVKFNWYV |
| | | | | DGVEVHNAKTKPCEEQYGSTYRCVSVLTVLHQDWLNG |
| | | | | KEYKCKVSNKALPAPIEKTISKAKGQPREPQVYTLPPSR |
| | | | | EEMTKNQVSLTCLVKGFYPSDIAVEWESNGQPENNYK |
| | | | | TTPPVLDSDGSFFLYSKLTVDKSRWQQGNVFSCSVMH |
| | | | | EALHNHYTQKSLSLSPGKGGGGSGGGGSGGGGSGG |
| | | | | GGSGGGGSGGGGSDKTHTCPPCPAPELLGGPSVFLF |
| | | | | PPKPKDTLMISRTPEVTCVVVDVSHEDPEVKFNWYVD |
| | | | | GVEVHNAKTKPCEEQYGSTYRCVSVLTVLHQDWLNGK |
| | | | | EYKCKVSNKALPAPIEKTISKAKGQPREPQVYTLPPSRE |
| | | | | EMTKNQVSLTCLVKGFYPSDIAVEWESNGQPENNYKT |
| | | | | TPPVLDSDGSFFLYSKLTVDKSRWQQGNVFSCSVMHE |
| | | | | ALHNHYTQKSLSLSPGK |
| 64. | MSLN_5_CCxCD3- | artificial | aa | QVQLVESGGGLVKPGGSLRLSCAASGFTFSDHYMSWI |
| | scFc Bispecific | | | RQAPGKCLEWFSYISSSGGIIYYADSVKGRFTISRDNAK |
| | HLE molecule | | | NSLYLQMNSLRAEDTAVYYCARDVGSHFDYWGQGTL |
| | | | | VTVSSGGGGSGGGGSGGGGSDIQMTQSPSSVSASVG |
| | | | | DRVTITCRASQDISRWLAWYQQKPGKAPKLLISAASRL |
| | | | | QSGVPSRFSGSGSGTDFTLTISSLQPEDFAIYYCQQAK |
| | | | | SFPRTFGCGTKVEIKSGGGGSEVQLVESGGGLVQPGG |
| | | | | SLKLSCAASGFTFNKYAMNWVRQAPGKGLEWVARIRS |
| | | | | KYNNYATYYADSVKDRFTISRDDSKNTAYLQMNNLKTE |
| | | | | DTAVYYCVRHGNFGNSYISYWAYWGQGTLVTVSSGG |
| | | | | GGSGGGGSGGGGSQTVVTQEPSLTVSPGGTVTLTCG |
| | | | | SSTGAVTSGNYPNWVQQKPGQAPRGLIGGTKFLAPGT |
| | | | | PARFSGSLLGGKAALTLSGVQPEDEAEYYCVLWYSNR |
| | | | | WVFGGGTKLTVLGGGGDKTHTCPPCPAPELLGGPSVF |
| | | | | LFPPKPKDTLMISRTPEVTCVVVDVSHEDPEVKFNWYV |
| | | | | DGVEVHNAKTKPCEEQYGSTYRCVSVLTVLHQDWLNG |
| | | | | KEYKCKVSNKALPAPIEKTISKAKGQPREPQVYTLPPSR |
| | | | | EEMTKNQVSLTCLVKGFYPSDIAVEWESNGQPENNYK |
| | | | | TTPPVLDSDGSFFLYSKLTVDKSRWQQGNVFSCSVMH |
| | | | | EALHNHYTQKSLSLSPGKGGGGSGGGGSGGGGSGG |
| | | | | GGSGGGGSGGGGSDKTHTCPPCPAPELLGGPSVFLF |
| | | | | PPKPKDTLMISRTPEVTCVVVDVSHEDPEVKFNWYVD |
| | | | | GVEVHNAKTKPCEEQYGSTYRCVSVLTVLHQDWLNGK |
| | | | | EYKCKVSNKALPAPIEKTISKAKGQPREPQVYTLPPSRE |
| | | | | EMTKNQVSLTCLVKGFYPSDIAVEWESNGQPENNYKT |
| | | | | TPPVLDSDGSFFLYSKLTVDKSRWQQGNVFSCSVMHE |
| | | | | ALHNHYTQKSLSLSPGK |
| 65. | CDR-H1 of | artificial | aa | SYGMH |
| | CDH19 | | | |
| | 65254.007 | | | |
| 66. | CDR-H2 of | artificial | aa | FIWYEGSNKYYAESVKD |
| | CDH19 | | | |
| | 65254.007 | | | |
| 67. | CDR-H3 of | artificial | aa | RAGIIGTIGYYYGMDV |
| | CDH19 | | | |
| | 65254.007 | | | |
| 68. | CDR-L1 of | artificial | aa | SGDRLGEKYTS |
| | CDH19 | | | |
| | 65254.007 | | | |
| 69. | CDR-L2 of | artificial | aa | QDTKRPS |
| | CDH19 | | | |
| | 65254.007 | | | |
| 70. | CDR-L3 of | artificial | aa | QAWESSTWV |
| | CDH19 | | | |
| | 65254.007 | | | |
| 71. | VH of CDH19 | artificial | aa | QVQLVESGGGVVQPGGSLRLSCAASGFTFSSYGMHW |
| | 65254.007 | | | VRQAPGKGLEWVAFIWYEGSNKYYAESVKDRFTISRD |
| | | | | NSKNTLYLQMNSLRAEDTAVYYCARRAGIIGTIGYYYG |
| | | | | MDVWGQGTTVTVSS |
| 72. | VL of CDH19 | artificial | aa | SYELTQPPSVSVSPGQTASITCSGDRLGEKYTSWYQQ |
| | 65254.007 | | | RPGQSPLLVIYQDTKRPSGIPERFSGSNSGNTATLTISG |
| | | | | TQAMDEADYYCQAWESSTVFGGGTKLTVLS |
| 73. | VH-VL of CDH19 | artificial | aa | QVQLVESGGGVVQPGGSLRLSCAASGFTFSSYGMHW |
| | 65254.007 | | | VRQAPGKGLEWVAFIWYEGSNKYYAESVKDRFTISRD |
| | | | | NSKNTLYLQMNSLRAEDTAVYYCARRAGIIGTIGYYYG |
| | | | | MDVWGQGTTVTVSSGGGGSGGGGSGGGGSSYELTQ |
| | | | | PPSVSVSPGQTASITCSGDRLGEKYTSWYQQRPGQSP |
| | | | | LLVIYQDTKRPSGIPERFSGSNSGNTATLTISGTQAMDE |
| | | | | ADYYCQAWESSTVVFGGGTKLTVLS |
| 74 | CDH19 | artificial | aa | QVQLVESGGGVVQPGGSLRLSCAASGFTFSSYGMHW |
| | 65254.007 x I2C | | | VRQAPGKGLEWVAFIWYEGSNKYYAESVKDRFTISRD |
| | | | | NSKNTLYLQMNSLRAEDTAVYYCARRAGIIGTIGYYYG |
| | | | | MDVWGQGTTVTVSSGGGGSGGGGSGGGGSSYELTQ |
| | | | | PPSVSVSPGQTASITCSGDRLGEKYTSWYQQRPGQSP |
| | | | | LLVIYQDTKRPSGIPERFSGSNSGNTATLTISGTQAMDE |
| | | | | ADYYCQAWESSTVVFGGGTKLTVLSGGGGSEVQLVES |
| | | | | GGGLVQPGGSLKLSCAASGFTFNKYAMNWVRQAPGK |
| | | | | GLEWVARIRSKYNNYATYYADSVKDRFTISRDDSKNTA |
| | | | | YLQMNNLKTEDTAVYYCVRHGNFGNSYISYWAYWGQ |
| | | | | GTLVTVSSGGGGSGGGGSGGGGSQTVVTQEPSLTVS |
| | | | | PGGTVTLTCGSSTGAVTSGNYPNWVQQKPGQAPRGLI |
| | | | | GGTKFLAPGTPARFSGSLLGGKAALTLSGVQPEDEAEY |
| | | | | YCVLWYSNRWVFGGGTKLTVLHHHHHH |
| 75. | CDH19 | artificial | aa | QVQLVESGGGWVQPGGSLRLSCAASGFTFSSYGMH |
| | 65254.007 x | | | WVRQAPGKGLEWVAFIWYEGSNKYYAESVKDRFTI |
| | I2C-scFc | | | SRDNSKNTLYLQMNSLRAEDTAVYYCARRAGIIGTIG |
| | Bispecific | | | YYYGMDVWGQGTTVTVSSGGGGSGGGGSGGGGS |
| | HLE molecule | | | SYELTQPPSVSVSPGQTASITCSGDRLGEKYTSWYQ |
| | | | | QRPGQSPLLVIYQDTKRPSGIPERFSGSNSGNTATL |
| | | | | TISGTQAMDEADYYCQAWESSTVVFGGGTKLTVLS |
| | | | | GGGGSEVQLVESGGGLVQPGGSLKLSCAASGFTFN |
| | | | | KYAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVT |
| | | | | SGNYPNWVQQKPGQAPRGLIGGTKFLAPGTPARFS |
| | | | | GSLLGGKAALTLSGVQPEDEAEYYCVLWYSNRWVF |
| | | | | GGGTKLTVLGGGGDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYT |
| | | | | LPPSREEMTKNQVSLTCLVKGFYPSDIAVEWESNGQ |
| | | | | PENNYKTTPPVLDSDGSFFLYSKLTVDKSRWQQGN |
| | | | | VFSCSVMHEALHNHYTQKSLSLSPGKGGGGSGGG |
| | | | | GSGGGGSGGGGSGGGGSGGGGSDKTHTCPPCPA |
| | | | | PELLGGPSVFLFPPKPKDTLMISRTPEVTCVVVDVSH |
| | | | | EDPEVKFNWYVDGVEVHNAKTKPCEEQYGSTYRCV |
| | | | | SVLTVLHQDWLNGKEYKCKVSNKALPAPIEKTISKAK |
| | | | | GQPREPQVYTLPPSREEMTKNQVSLTCLVKGFYPS |
| | | | | DIAVEWESNGQPENNYKTTPPVLDSDGSFFLYSKLT |
| | | | | VDKSRWQQGNVFSCSVMHEALHNHYTQKSLSLSPG |
| | | | | K |
| 76. | CDH19 | artificial | aa | QVQLVESGGGWQPGGSLRLSCAASGFTFSSYGM |
| | 65254.007 x I2C | | | HWVRQAPGKGLEWVAFIWYEGSNKYYAESVKDRFT |
| | -scFc_delGK | | | ISRDNSKNTLYLQMNSLRAEDTAVYYCARRAGIIGTI |
| | Bispecific HLE | | | GYYYGMDVWGQGTTVTVSSGGGGSGGGGSGGGG |
| | molecule | | | SSYELTQPPSVSVSPGQTASITCSGDRLGEKYTSWY |
| | | | | QQRPGQSPLLVIYQDTKRPSGIPERFSGSNSGNTAT |
| | | | | LTISGTQAMDEADYYCQAWESSTWFGGGTKLTVLS |
| | | | | GGGGSEVQLVESGGGLVQPGGSLKLSCAASGFTFN |
| | | | | KYAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVT |
| | | | | SGNYPNWVQQKPGQAPRGLIGGTKFLAPGTPARFS |
| | | | | GSLLGGKAALTLSGVQPEDEAEYYCVLWYSNRWVF |
| | | | | GGGTKLTVLGGGGDKTHTCPPCPAPELLGGPSVFL |
| | | | | FPPKPKDTLMISRTPEVTCVWVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYT |
| | | | | LPPSREEMTKNQVSLTCLVKGFYPSDIAVEWESNG |
| | | | | QPENNYKTTPPVLDSDGSFFLYSKLTVDKSRWQQG |
| | | | | NVFSCSVMHEALHNHYTQKSLSLSPGGGGSGGGG |
| | | | | SGGGGSGGGGSGGGGSGGGGSDKTHTCPPCPAP |
| | | | | ELLGGPSVFLFPPKPKDTLMISRTPEVTCVVVDVSHE |
| | | | | DPEVKFNWYVDGVEVHNAKTKPCEEQYGSTYRCVS |
| | | | | VLTVLHQDWLNGKEYKCKVSNKALPAPIEKTISKAKG |
| | | | | QPREPQVYTLPPSREEMTKNQVSLTCLVKGFYPSDI |
| | | | | AVEWESNGQPENNYKTTPPVLDSDGSFFLYSKLTV |
| | | | | DKSRWQQGNVFSCSVMHEALHNHYTQKSLSLSPG |
| | | | | K |
| 77. | CDH19 | artificial | aa | QVQLVESGGGWQPGGSLRLSCAASGFTFSSYGM |
| | 65254.007_CC x | | | HWVRQAPGKCLEWVAFIWYEGSNKYYAESVKDRFT |
| | I2C-scFc VH | | | ISRDNSKNTLYLQMNSLRAEDTAVYYCARRAGIIGTI |
| | | | | GYYYGMDVWGQGTTVTVSS |
| 78. | CDH19 | artificial | aa | SYELTQPPSVSVSPGQTASITCSGDRLGEKYTSWY |
| | 65254.007_CC x | | | QQRPGQSPLLVIYQDTKRPSGIPERFSGSNSGNTAT |
| | I2C-scFc VL | | | LTISGTQAMDEADYYCQAWESSTVVFGCGTKLTVL |
| 79. | CDH19 | artificial | aa | QVQLVESGGGVVQPGGSLRLSCAASGFTFSSYGM |
| | 65254.007_CC x | | | HWVRQAPGKCLEWVAFIWYEGSNKYYAESVKDRFT |
| | I2C-scFc scFv | | | ISRDNSKNTLYLQMNSLRAEDTAVYYCARRAGIIGTI |
| | | | | GYYYGMDVWGQGTTVTVSSGGGGSGGGGSGGGG |
| | | | | SSYELTQPPSVSVSPGQTASITCSGDRLGEKYTSWY |
| | | | | QQRPGQSPLLVIYQDTKRPSGIPERFSGSNSGNTAT |
| | | | | LTISGTQAMDEADYYCQAWESSTVVFGCGTKLTVL |
| 80. | CDH19 | artificial | aa | QVQLVESGGGVVQPGGSLRLSCAASGFTFSSYGM |
| | 65254.007_CC x | | | HWVRQAPGKCLEWVAFIWYEGSNKYYAESVKDRFT |
| | I2C-scFc | | | ISRDNSKNTLYLQMNSLRAEDTAVYYCARRAGIIGTI |
| | Bispecific | | | GYYYGMDVWGQGTTVTVSSGGGGSGGGGSGGGG |
| | molecule | | | SSYELTQPPSVSVSPGQTASITCSGDRLGEKYTSWY |
| | | | | QQRPGQSPLLVIYQDTKRPSGIPERFSGSNSGNTAT |
| | | | | LTISGTQAMDEADYYCQAWESSTVFGCGTKLTVLS |
| | | | | GGGGSEVQLVESGGGLVQPGGSLKLSCAASGFTFN |
| | | | | KYAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVT |
| | | | | SGNYPNWVQQKPGQAPRGLIGGTKFLAPGTPARFS |
| | | | | GSLLGGKAALTLSGVQPEDEAEYYCVLWYSNRWVF |
| | | | | GGGTKLTVL |
| 81. | CDH19 | artificial | aa | QVQLVESGGGVVQPGGSLRLSCAASGFTFSSYGM |
| | 65254.007_CC x | | | HWVRQAPGKCLEWVAFIWYEGSNKYYAESVKDRFT |
| | I2C-scFc | | | ISRDNSKNTLYLQMNSLRAEDTAVYYCARRAGIIGTI |
| | Bispecific HLE | | | GYYYGMDVWGQGTTVTVSSGGGGSGGGGSGGGG |
| | molecule | | | SSYELTQPPSVSVSPGQTASITCSGDRLGEKYTSWY |
| | | | | QQRPGQSPLLVIYQDTKRPSGIPERFSGSNSGNTAT |
| | | | | LTISGTQAMDEADYYCQAWESSTWFGCGTKLTVLS |
| | | | | GGGGSEVQLVESGGGLVQPGGSLKLSCAASGFTFN |
| | | | | KYAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVT |
| | | | | SGNYPNWVQQKPGQAPRGLIGGTKFLAPGTPARFS |
| | | | | GSLLGGKAALTLSGVQPEDEAEYYCVLWYSNRWVF |
| | | | | GGGTKLTVLGGGGDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYT |
| | | | | LPPSREEMTKNQVSLTCLVKGFYPSDIAVEWESNG |
| | | | | QPENNYKTTPPVLDSDGSFFLYSKLTVDKSRWQQG |
| | | | | NVFSCSVMHEALHNHYTQKSLSLSPGKGGGGSGG |
| | | | | GGSGGGGSGGGGSGGGGSGGGGSDKTHTCPPCP |
| | | | | APELLGGPSVFLFPPKPKDTLMISRTPEVTCVVVDVS |
| | | | | HEDPEVKFNWYVDGVEVHNAKTKPCEEQYGSTYR |
| | | | | CVSVLTVLHQDWLNGKEYKCKVSNKALPAPIEKTISK |
| | | | | AKGQPREPQVYTLPPSREEMTKNQVSLTCLVKGFY |
| | | | | PSDIAVEWESNGQPENNYKTTPPVLDSDGSFFLYSK |
| | | | | LTVDKSRWQQGNVFSCSVMHEALHNHYTQKSLSLS |
| | | | | PGK |
| 82. | CDH19 | artificial | aa | QVQLVESGGGVVQPGGSLRLSCAASGFTFSSYGM |
| | 65254.007_CC x | | | HWVRQAPGKCLEWVAFIWYEGSNKYYAESVKDRFT |
| | I2C- | | | ISRDNSKNTLYLQMNSLRAEDTAVYYCARRAGIIGTI |
| | scFc_delGK | | | GYYYGMDVWGQGTTVTVSSGGGGSGGGGSGGGG |
| | Bispecific HLE | | | SSYELTQPPSVSVSPGQTASITCSGDRLGEKYTSWY |
| | molecule | | | QQRPGQSPLLVIYQDTKRPSGIPERFSGSNSGNTAT |
| | | | | LTISGTQAMDEADYYCQAWESSTVVFGCGTKLTVLS |
| | | | | GGGGSEVQLVESGGGLVQPGGSLKLSCAASGFTFN |
| | | | | KYAMNWVRQAPGKGLEWVARIRSKYNNYATYYADS |
| | | | | VKDRFTISRDDSKNTAYLQMNNLKTEDTAVYYCVRH |
| | | | | GNFGNSYISYWAYWGQGTLVTVSSGGGGSGGGGS |
| | | | | GGGGSQTVVTQEPSLTVSPGGTVTLTCGSSTGAVT |
| | | | | SGNYPNWVQQKPGQAPRGLIGGTKFLAPGTPARFS |
| | | | | GSLLGGKAALTLSGVQPEDEAEYYCVLWYSNRWVF |
| | | | | GGGTKLTVLGGGGDKTHTCPPCPAPELLGGPSVFL |
| | | | | FPPKPKDTLMISRTPEVTCVVVDVSHEDPEVKFNWY |
| | | | | VDGVEVHNAKTKPCEEQYGSTYRCVSVLTVLHQDW |
| | | | | LNGKEYKCKVSNKALPAPIEKTISKAKGQPREPQVYT |
| | | | | LPPSREEMTKNQVSLTCLVKGFYPSDIAVEWESNG |
| | | | | QPENNYKTTPPVLDSDGSFFLYSKLTVDKSRWQQG |
| | | | | NVFSCSVMHEALHNHYTQKSLSLSPGGGGSGGGG |
| | | | | SGGGGSGGGGSGGGGSGGGGSDKTHTCPPCPAP |
| | | | | ELLGGPSVFLFPPKPKDTLMISRTPEVTCVVVDVSHE |
| | | | | DPEVKFNWYVDGVEVHNAKTKPCEEQYGSTYRCVS |
| | | | | VLTVLHQDWLNGKEYKCKVSNKALPAPIEKTISKAKG |
| | | | | QPREPQVYTLPPSREEMTKNQVSLTCLVKGFYPSDI |
| | | | | AVEWESNGQPENNYKTTPPVLDSDGSFFLYSKLTV |
| | | | | DKSRWQQGNVFSCSVMHEALHNHYTQKSLSLSPG |
| | | | | K |
| 83. | FLT3_7 | artificial | aa | NARMGVS |
| | A8xCD3-scFc | | | |
| | VH CDR1 | | | |
| 84. | FLT3_7 | artificial | aa | HIFSNDEKSYSTSLKN |
| | A8xCD3-scFc | | | |
| | VH CDR2 | | | |
| 85. | FLT3_7 | artificial | aa | IVGYGSGWYGFFDY |
| | A8xCD3-scFc | | | |
| | VH CDR3 | | | |
| 86. | FLT3_7 | artificial | aa | RASQGIRNDLG |
| | A8xCD3-scFc | | | |
| | VL CDR1 | | | |
| 87. | FLT3_7 | artificial | aa | AASTLOS |
| | A8xCD3-scFc | | | |
| | VL CDR2 | | | |
| 88. | FLT3_7 | artificial | aa | LQHNSYPLT |
| | A8xCD3-scFc | | | |
| | VL CDR3 | | | |
| 89. | FLT3_7 | artificial | aa | QVTLKESGPTLVKPTETLTLTCTLSGFSLNNARMGVS |
| | A8xCD3-scFc | | | WIRQPPGKCLEWLAHIFSNDEKSYSTSLKNRLTISKDS |
| | VH | | | SKTQWVLTMTNVDPVDTATYYCARIVGYGSGWYGFFD |
| | | | | YWGQGTLVTVSS |
| 90. | FLT3_A8- | artificial | aa | DIQMTQSPSSLSASVGDRVTITCRASQGIRNDLGWYQ |
| | scFc VL | | | QKPGKAPKRLIYAASTLQSGVPSRFSGSGSGTEFTLTI |
| | | | | SSLQPEDFATYYCLQHNSYPLTFGCGTKVEIK |
| 91. | FLT3_7 | artificial | aa | QVTLKESGPTLVKPTETLTLTCTLSGFSLNNARMGVS |
| | A8xCD3-scFv | | | WIRQPPGKCLEWLAHIFSNDEKSYSTSLKNRLTISKDS |
| | | | | SKTQVVLTMTNVDPVDTATYYCARIVGYGSGWYGFFD |
| | | | | YWGQGTLVTVSSGGGGSGGGGSGGGGSDIQMTQSP |
| | | | | SSLSASVGDRVTITCRASQGIRNDLGWYQQKPGKAPK |
| | | | | RLIYAASTLQSGVPSRFSGSGSGTEFTLTISSLQPEDF |
| | | | | ATYYCLQHNSYPLTFGCGTKVEIK |
| 92. | FLT3_7 | artificial | aa | QVTLKESGPTLVKPTETLTLTCTLSGFSLNNARMGVS |
| | A8xCD3 | | | WIRQPPGKCLEWLAHIFSNDEKSYSTSLKNRLTISKDS |
| | Bispecific | | | SKTQVVLTMTNVDPVDTATYYCARIVGYGSGWYGFFD |
| | molecule | | | YWGQGTLVTVSSGGGGSGGGGSGGGGSDIQMTQSP |
| | | | | SSLSASVGDRVTITCRASQGIRNDLGWYQQKPGKAPK |
| | | | | RLIYAASTLQSGVPSRFSGSGSGTEFTLTISSLQPEDF |
| | | | | ATYYCLQHNSYPLTFGCGTKVEIKSGGGGSEVQLVES |
| | | | | GGGLVQPGGSLKLSCAASGFTFNKYAMNWVRQAPGK |
| | | | | GLEWVARIRSKYNNYATYYADSVKDRFTISRDDSKNTA |
| | | | | YLQMNNLKTEDTAVYYCVRHGNFGNSYISYWAYWGQ |
| | | | | GTLVTVSSGGGGSGGGGSGGGGSQTVVTQEPSLTVS |
| | | | | PGGTVTLTCGSSTGAVTSGNYPNWVQQKPGQAPRGL |
| | | | | IGGTKFLAPGTPARFSGSLLGGKAALTLSGVQPEDEAE |
| | | | | YYCVLWYSNRWVFGGGTKLTVL |
| 93. | FLT3_7 | artificial | aa | QVTLKESGPTLVKPTETLTLTCTLSGFSLNNARMGVS |
| | A8xCD3-scFc | | | WIRQPPGKCLEWLAHIFSNDEKSYSTSLKNRLTISKDS |
| | Bispecific HLE | | | SKTQVVLTMTNVDPVDTATYYCARIVGYGSGWYGFFD |
| | molecule | | | YWGQGTLVTVSSGGGGSGGGGSGGGGSDIQMTQSP |
| | | | | SSLSASVGDRVTITCRASQGIRNDLGWYQQKPGKAPK |
| | | | | RLIYAASTLQSGVPSRFSGSGSGTEFTLTISSLQPEDF |
| | | | | ATYYCLQHNSYPLTFGCGTKVEIKSGGGGSEVQLVES |
| | | | | GGGLVQPGGSLKLSCAASGFTFNKYAMNWVRQAPGK |
| | | | | GLEWVARIRSKYNNYATYYADSVKDRFTISRDDSKNTA |
| | | | | YLQMNNLKTEDTAVYYCVRHGNFGNSYISYWAYWGQ |
| | | | | GTLVTVSSGGGGSGGGGSGGGGSQTVVTQEPSLTVS |
| | | | | PGGTVTLTCGSSTGAVTSGNYPNWVQQKPGQAPRGL |
| | | | | IGGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 94. | VH CDR1 | artificial | aa | SYYWS |
| | DLL3_1_CC_ | | | |
| | delGK | | | |
| 95. | VH CDR2 | artificial | aa | YVYYSGTTNYNPSLKS |
| | DLL3_1_CC_ | | | |
| | delGK | | | |
| 96. | VH CDR3 | artificial | aa | IAVTGFYFDY |
| | DLL3_1_CC_ | | | |
| | delGK | | | |
| 97. | VL CDR1 | artificial | aa | RASQRVNNNYLA |
| | DLL3_1_CC_ | | | |
| | delGK | | | |
| 98. | VL CDR2 | artificial | aa | GASSRAT |
| | DLL3_1_CC_ | | | |
| | delGK | | | |
| 99. | VL CDR3 | artificial | aa | QQYDRSPLT |
| | DLL3_1_CC_ | | | |
| | delGK | | | |
| 100. | VH | artificial | aa | QVQLQESGPGLVKPSETLSLTCTVSGGSISSYYWSWI |
| | DLL3_1_CC_ | | | RQPPGKCLEWIGYVYYSGTTNYNPSLKSRVTISVDTSK |
| | delGK | | | NQFSLKLSSVTAADTAVYYCASIAVTGFYFDYWGQGT |
| | | | | LVTVSS |
| 101. | VL | artificial | aa | EIVLTQSPGTLSLSPGERVTLSCRASQRVNNNYLAWY |
| | DLL3_1_CC_ | | | QQRPGQAPRLLIYGASSRATGIPDRFSGSGSGTDFTL |
| | delGK | | | TISRLEPEDFAVYYCQQYDRSPLTFGCGTKLEIK |
| 102. | DLL3_1_CC_ | artificial | aa | QVQLQESGPGLVKPSETLSLTCTVSGGSISSYYWSWI |
| | delGK | | | RQPPGKCLEWIGYVYYSGTTNYNPSLKSRVTISVDTSK |
| | | | | NQFSLKLSSVTAADTAVYYCASIAVTGFYFDYWGQGT |
| | | | | LVTVSSGGGGSGGGGSGGGGSEIVLTQSPGTLSLSP |
| | | | | GERVTLSCRASQRVNNNYLAWYQQRPGQAPRLLIYG |
| | | | | ASSRATGIPDRFSGSGSGTDFTLTISRLEPEDFAVYYC |
| | | | | QQYDRSPLTFGCGTKLEIK |
| 103. | DLL3_1_CCxCD3_ | artificial | aa | QVQLQESGPGLVKPSETLSLTCTVSGGSISSYYWSWI |
| | delGK | | | RQPPGKCLEWIGYVYYSGTTNYNPSLKSRVTISVDTSK |
| | Bispecific | | | NQFSLKLSSVTAADTAVYYCASIAVTGFYFDYWGQGT |
| | molecule | | | LVTVSSGGGGSGGGGSGGGGSEIVLTQSPGTLSLSP |
| | | | | GERVTLSCRASQRVNNNYLAWYQQRPGQAPRLLIYG |
| | | | | ASSRATGIPDRFSGSGSGTDFTLTISRLEPEDFAVYYC |
| | | | | QQYDRSPLTFGCGTKLEIKSGGGGSEVQLVESGGGLV |
| | | | | QPGGSLKLSCAASGFTFNKYAMNWVRQAPGKGLEW |
| | | | | VARIRSKYNNYATYYADSVKDRFTISRDDSKNTAYLQM |
| | | | | NNLKTEDTAVYYCVRHGNFGNSYISYWAYWGQGTLV |
| | | | | TVSSGGGGSGGGGSGGGGSQTVVTQEPSLTVSPGG |
| | | | | TVTLTCGSSTGAVTSGNYPNWVQQKPGQAPRGLIGG |
| | | | | TKFLAPGTPARFSGSLLGGKAALTLSGVQPEDEAEYY |
| | | | | CVLWYSNRWVFGGGTKLTVL |
| 104. | DLL3_1_CCxCD3- | artificial | aa | QVQLQESGPGLVKPSETLSLTCTVSGGSISSYYWSWI |
| | scFc_delGK | | | RQPPGKCLEWIGYVYYSGTTNYNPSLKSRVTISVDTSK |
| | Bispecific HLE | | | NQFSLKLSSVTAADTAVYYCASIAVTGFYFDYWGQGT |
| | molecule | | | LVTVSSGGGGSGGGGSGGGGSEIVLTQSPGTLSLSP |
| | | | | GERVTLSCRASQRVNNNYLAWYQQRPGQAPRLLIYG |
| | | | | ASSRATGIPDRFSGSGSGTDFTLTISRLEPEDFAVYYC |
| | | | | QQYDRSPLTFGCGTKLEIKSGGGGSEVQLVESGGGLV |
| | | | | QPGGSLKLSCAASGFTFNKYAMNWVRQAPGKGLEW |
| | | | | VARIRSKYNNYATYYADSVKDRFTISRDDSKNTAYLQM |
| | | | | NNLKTEDTAVYYCVRHGNFGNSYISYWAYWGQGTLV |
| | | | | TVSSGGGGGGGGSGGGGSQTVVTQEPSLTVSPGG |
| | | | | TVTLTCGSSTGAVTSGNYPNWVQQKPGQAPRGLIGG |
| | | | | TKFLAPGTPARFSGSLLGGKAALTLSGVQPEDEAEYY |
| | | | | CVLWYSNRWVFGGGTKLTVLGGGGDKTHTCPPCPAP |
| | | | | ELLGGPSVFLFPPKPKDTLMISRTPEVTCWVDVSHED |
| | | | | PEVKFNWYVDGVEVHNAKTKPCEEQYGSTYRCVSVL |
| | | | | TVLHQDWLNGKEYKCKVSNKALPAPIEKTISKAKGQPR |
| | | | | EPQVYTLPPSREEMTKNQVSLTCLVKGFYPSDIAVEW |
| | | | | ESNGQPENNYKTTPPVLDSDGSFFLYSKLTVDKSRWQ |
| | | | | QGNVFSCSVMHEALHNHYTQKSLSLSPGGGGSGGG |
| | | | | GSGGGGSGGGGSGGGGSGGGGSDKTHTCPPCPAP |
| | | | | ELLGGPSVFLFPPKPKDTLMISRTPEVTCVVDVSHED |
| | | | | PEVKFNWYVDGVEVHNAKTKPCEEQYGSTYRCVSVL |
| | | | | TVLHQDWLNGKEYKCKVSNKALPAPIEKTISKAKGQPR |
| | | | | EPQVYTLPPSREEMTKNQVSLTCLVKGFYPSDIAVEW |
| | | | | ESNGQPENNYKTTPPVLDSDGSFFLYSKLTVDKSRWQ |
| | | | | QGNVFSCSVMHEALHNHYTQKSLSLSPGK |
| 105 | VH CDR1 | artificial | aa | SYGMH |
| | CD19 97- | | | |
| | G1RE-C2 | | | |
| 106. | VH CDR2 | artificial | aa | VISYEGSNKYYAESVKG |
| | CD19 97- | | | |
| | G1RE-C2 | | | |
| 107 | VH CDR3 | artificial | aa | DRGTIFGNYGLEV |
| | CD19 97- | | | |
| | G1RE-C2 | | | |
| 108. | VH CD19 97- | artificial | aa | QVQLVESGGGWQPGRSLRLSCAASGFTFSSYGMHW |
| | G1RE-C2 CC | | | VRQAPGKCLEWVAVISYEGSNKYYAESVKGRFTISRD |
| | | | | NSKNTLYLQMNSLRDEDTAVYYCARDRGTIFGNYGLE |
| | | | | VWGQGTTVTVSS |
| 109. | VL CDR1 | artificial | aa | RSSQSLLHKNAFNYLD |
| | CD19 97- | | | |
| | G1RE-C2 | | | |
| 110. | VL CDR2 | artificial | aa | LGSNRAS |
| | CD19 97- | | | |
| | G1RE-C2 | | | |
| 111. | VL CDR3 | artificial | aa | MQALQTPFT |
| | CD19 97- | | | |
| | G1RE-C2 | | | |
| 112 | VL CD 19 97- | artificial | aa | DIVMTQSPLSLPVISGEPASISCRSSQSLLHKNAFNYLD |
| | G1RE-C2 CC | | | WYLQKPGQSPQLLIYLGSNRASGVPDRFSGSGSGTD |
| | | | | FTLKISRVEAEDVGVYYCMQALQTPFTFGCGTKVDIK |
| 113 | CD19 97- | artificial | aa | MDMRVPAQLLGLLLLWLRGARCDIVMTQSPLSLPVISG |
| | G1RE-C2 CC | | | EPASISCRSSQSLLHKNAFNYLDWYLQKPGQSPQLLIY |
| | x I2C0 | | | LGSNRASGVPDRFSGSGSGTDFTLKISRVEAEDVGVY |
| | | | | YCMQALQTPFTFGCGTKVDIKGGGGSGGGGSGGGG |
| | | | | SQVQLVESGGGVVQPGRSLRLSCAASGFTFSSYGMH |
| | | | | WVRQAPGKCLEWVAVISYEGSNKYYAESVKGRFTISR |
| | | | | DNSKNTLYLQMNSLRDEDTAVYYCARDRGTIFGNYGL |
| | | | | EVWGQGTTVTVSSGGGGSEVQLVESGGGLVQPGGS |
| | | | | LKLSCAASGFTFNKYAMNWVRQAPGKGLEWVARIRS |
| | | | | KYNNYATYYADSVKDRFTISRDDSKNTAYLQMNNLKT |
| | | | | EDTAVYYCVRHGNFGNSYISYWAYWGQGTLVTVSSG |
| | | | | GGGSGGGGSGGGGSQTVVTQEPSLTVSPGGTVTLTC |
| | | | | GSSTGAVTSGNYPNWVQQKPGQAPRGLIGGTKFLAP |
| | | | | GTPARFSGSLLGGKAALTLSGVQPEDEAEYYCVLWYS |
| | | | | NRWVFGGGTKLTVL |
| 114. | CD19 97- | artificial | aa | MDMRVPAQLLGLLLLWLRGARCDIVMTQSPLSLPVISG |
| | G1RE-C2 CC | | | EPASISCRSSQSLLHKNAFNYLDWYLQKPGQSPQLLIY |
| | x I2C0-scFc | | | LGSNRASGVPDRFSGSGSGTDFTLKISRVEAEDVGVY |
| | | | | YCMQALQTPFTFGCGTKVDIKGGGGSGGGGSGGGG |
| | | | | SQVQLVESGGGVVQPGRSLRLSCAASGFTFSSYGMH |
| | | | | WVRQAPGKCLEWVAVISYEGSNKYYAESVKGRFTISR |
| | | | | DNSKNTLYLQMNSLRDEDTAVYYCARDRGTIFGNYGL |
| | | | | EVWGQGTTVTVSSGGGGSEVQLVESGGGLVQPGGS |
| | | | | LKLSCAASGFTFNKYAMNWVRQAPGKGLEWVARIRS |
| | | | | KYNNYATYYADSVKDRFTISRDDSKNTAYLQMNNLKT |
| | | | | EDTAVYYCVRHGNFGNSYISYWAYWGQGTLVTVSSG |
| | | | | GGGSGGGGSGGGGSQTVVTQEPSLTVSPGGTVTLTC |
| | | | | GSSTGAVTSGNYPNWVQQKPGQAPRGLIGGTKFLAP |
| | | | | GTPARFSGSLLGGKAALTLSGVQPEDEAEYYCVLWYS |
| | | | | NRWVFGGGTKLTVLGGGGDKTHTCPPCPAPELLGGP |
| | | | | SVFLFPPKPKDTLMISRTPEVTCVVVDVSHEDPEVKFN |
| | | | | WYVDGVEVHNAKTKPCEEQYGSTYRCVSVLTVLHQD |
| | | | | WLNGKEYKCKVSNKALPAPIEKTISKAKGQPREPQVYT |
| | | | | LPPSREEMTKNQVSLTCLVKGFYPSDIAVEWESNGQP |
| | | | | ENNYKTTPPVLDSDGSFFLYSKLTVDKSRWQQGNVFS |
| | | | | CSVMHEALHNHYTQKSLSLSPGKGGGGSGGGGSGG |
| | | | | GGSGGGGSGGGGSGGGGSDKTHTCPPCPAPELLGG |
| | | | | PSVFLFPPKPKDTLMISRTPEVTCVWVDVSHEDPEVKF |
| | | | | NWYVDGVEVHNAKTKPCEEQYGSTYRCVSVLTVLHQ |
| | | | | DWLNGKEYKCKVSNKALPAPIEKTISKAKGQPREPQV |
| | | | | YTLPPSREEMTKNQVSLTCLVKGFYPSDIAVEWESNG |
| | | | | QPENNYKTTPPVLDSDGSFFLYSKLTVDKSRWQQGN |
| | | | | VFSCSVMHEALHNHYTQKSLSLSPGK |
| 115. | VH CDR1 | artificial | aa | SYPIN |
| | CDH3 G8A 6-B12 | | | |
| 116. | VH CDR2 | artificial | aa | VIWTGGGTNYASSVKG |
| | CDH3 G8A 6-B12 | | | |
| 117. | VH CDR3 | artificial | aa | SRGVYDFDGRGAMDY |
| | CDH3 G8A 6-B12 | | | |
| 118. | VL CDR1 | artificial | aa | KSSQSLLYSSNQKNYFA |
| | CDH3 G8A 6-B12 | | | |
| 119 | VL CDR2 | artificial | aa | WASTRES |
| | CDH3 G8A 6-B12 | | | |
| 120. | VL CDR3 | artificial | aa | QQYYSYPYT |
| | CDH3 G8A 6-B12 | | | |
| 121. | VH CDH3 G8A | artificial | aa | EVQLLESGGGLVQPGGSLRLSCAASGFSFSSYPINWV |
| | 6-B12 | | | RQAPGKGLEWVGVIWTGGGTNYASSVKGRFTISRDN |
| | | | | SKNTVYLQMNSLRAEDTAVYYCAKSRGVYDFDGRGA |
| | | | | MDYWGQGTLVTVSS |
| 122. | VL CDH3 G8A | artificial | aa | DIVMTQSPDSLAVSLGERATINCKSSQSLLYSSNQKNY |
| | 6-B12 | | | FAWYQQKPGQPPKLLIYWASTRESGVPDRFSGSGSG |
| | | | | TDFTLTISSLQAEDVAVYYCQQYYSYPYTFGQGTKLEI |
| | | | | K |
| 123. | CDH3 G8A 6- | artificial | aa | EVQLLESGGGLVQPGGSLRLSCAASGFSFSSYPINWV |
| | B12 scFv | | | RQAPGKGLEWVGVIWTGGGTNYASSVKGRFTISRDN |
| | | | | SKNTVYLQMNSLRAEDTAVYYCAKSRGVYDFDGRGA |
| | | | | MDYWGQGTLVTVSSGGGGSGGGGSGGGGSDIVMTQ |
| | | | | SPDSLAVSLGERATINCKSSQSLLYSSNQKNYFAWYQ |
| | | | | QKPGQPPKLLIYWASTRESGVPDRFSGSGSGTDFTLTI |
| | | | | SSLQAEDVAVYYCQQYYSYPYTFGQGTKLEIK |
| 124. | CDH3 G8A 6- | artificial | aa | EVQLLESGGGLVQPGGSLRLSCAASGFSFSSYPINWV |
| | B12 x I2C0 | | | RQAPGKGLEWVGVIWTGGGTNYASSVKGRFTISRDN |
| | bispecific | | | SKNTVYLQMNSLRAEDTAVYYCAKSRGVYDFDGRGA |
| | molecule | | | MDYWGQGTLVTVSSGGGGSGGGGSGGGGSDIVMTQ |
| | | | | SPDSLAVSLGERATINCKSSQSLLYSSNQKNYFAWYQ |
| | | | | QKPGQPPKLLIYWASTRESGVPDRFSGSGSGTDFTLTI |
| | | | | SSLQAEDVAVYYCQQYYSYPYTFGQGTKLEIKSGGGG |
| | | | | SEVQLVESGGGLVQPGGSLKLSCAASGFTFNKYAMN |
| | | | | WVRQAPGKGLEWVARIRSKYNNYATYYADSVKDRFTI |
| | | | | SRDDSKNTAYLQMNNLKTEDTAVYYCVRHGNFGNSYI |
| | | | | SYWAYWGQGTLVTVSSGGGGSGGGGSGGGGSQTV |
| | | | | VTQEPSLTVSPGGTVTLTCGSSTGAVTSGNYPNWVQ |
| | | | | QKPGQAPRGLIGGTKFLAPGTPARFSGSLLGGKAALT |
| | | | | LSGVQPEDEAEYYCVLWYSNRWVFGGGTKLTVL |
| 125. | CDH3 G8A 6- | artificial | aa | EVQLLESGGGLVQPGGSLRLSCAASGFSFSSYPINWV |
| | B12 x I2C0 | | | RQAPGKGLEWVGVIWTGGGTNYASSVKGRFTISRDN |
| | bispecific | | | SKNTVYLQMNSLRAEDTAVYYCAKSRGVYDFDGRGA |
| | molecule HLE | | | MDYWGQGTLVTVSSGGGGSGGGGSGGGGSDIVMTQ |
| | | | | SPDSLAVSLGERATINCKSSQSLLYSSNQKNYFAWYQ |
| | | | | QKPGQPPKLLIYWASTRESGVPDRFSGSGSGTDFTLTI |
| | | | | SSLQAEDVAVYYCQQYYSYPYTFGQGTKLEIKSGGGG |
| | | | | SEVQLVESGGGLVQPGGSLKLSCAASGFTFNKYAMN |
| | | | | WVRQAPGKGLEWVARIRSKYNNYATYYADSVKDRFTI |
| | | | | SRDDSKNTAYLQMNNLKTEDTAVYYCVRHGNFGNSYI |
| | | | | SYWAYWGQGTLVTVSSGGGGSGGGGSGGGGSQTV |
| | | | | VTQEPSLTVSPGGTVTLTCGSSTGAVTSGNYPNWVQ |
| | | | | QKPGQAPRGLIGGTKFLAPGTPARFSGSLLGGKAALT |
| | | | | LSGVQPEDEAEYYCVLWYSNRWVFGGGTKLTVLGGG |
| | | | | GDKTHTCPPCPAPELLGGPSVFLFPPKPKDTLMISRTP |
| | | | | EVTCVWVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLTC |
| | | | | LVKGFYPSDIAVEWESNGQPENNYKTTPPVLDSDGSF |
| | | | | FLYSKLTVDKSRWQQGNVFSCSVMHEALHNHYTQKS |
| | | | | LSLSPGKGGGGSGGGGSGGGGSGGGGSGGGGSGG |
| | | | | GGSDKTHTCPPCPAPELLGGPSVFLFPPKPKDTLMISR |
| | | | | TPEVTCVVVDVSHEDPEVKFNWYVDGVEVHNAKTKP |
| | | | | CEEQYGSTYRCVSVLTVLHQDWLNGKEYKCKVSNKA |
| | | | | LPAPIEKTISKAKGQPREPQVYTLPPSREEMTKNQVSL |
| | | | | TCLVKGFYPSDIAVEWESNGQPENNYKTTPPVLDSDG |
| | | | | SFFLYSKLTVDKSRWQQGNVFSCSVMHEALHNHYTQ |
| | | | | KSLSLSPGK |
| 126. | BCMA A7 27- | artificial | aa | NHIIH |
| | C4-G7 CDR1 | | | |
| | VH | | | |
| 127. | BCMA A7 27- | artificial | aa | YINPYPGYHAYNEKFQG |
| | C4-G7 CDR2 | | | |
| | VH | | | |
| 128. | BCMA A7 27- | artificial | aa | DGYYRDTDVLDY |
| | C4-G7 CDR3 | | | |
| | VH | | | |
| 129. | BCMA A7 27- | artificial | aa | QASQDISNYLN |
| | C4-G7 CDR1 | | | |
| | VL | | | |
| 130. | BCMA A7 27- | artificial | aa | YTSRLHT |
| | C4-G7 CDR2 | | | |
| | VL | | | |
| 131. | BCMA A7 27- | artificial | aa | QQGNTLPWT |
| | C4-G7 CDR3 | | | |
| | VL | | | |
| 132. | BCMA A7 27- | artificial | aa | QVQLVQSGAEVKKPGASVKVSCKASGYTFTNHIIHWV |
| | C4-G7 CC | | | RQAPGQCLEWMGYINPYPGYHAYNEKFQGRATMTSD |
| | (44/100) VH | | | TSTSTVYMELSSLRSEDTAVYYCARDGYYRDTDVLDY |
| | | | | WGQGTLVTVSS |
| 133. | BCMA A7 27- | artificial | aa | DIQMTQSPSSLSASVGDRVTITCQASQDISNYLNWYQ |
| | C4-G7 CC | | | QKPGKAPKLLIYYTSRLHTGVPSRFSGSGSGTDFTFTI |
| | (44/100) VL | | | SSLEPEDIATYYCQQGNTLPWTFGCGTKLEIK |
| 134. | BCMA A7 27- | artificial | aa | QVQLVQSGAEVKKPGASVKVSCKASGYTFTNHIIHWV |
| | C4-G7 CC | | | RQAPGQCLEWMGYINPYPGYHAYNEKFQGRATMTSD |
| | (44/100) scFv | | | TSTSTVYMELSSLRSEDTAVYYCARDGYYRDTDVLDY |
| | | | | WGQGTLVTVSSGGGGSGGGGSGGGGSDIQMTQSPS |
| | | | | SLSASVGDRVTITCQASQDISNYLNWYQQKPGKAPKL |
| | | | | LIYYTSRLHTGVPSRFSGSGSGTDFTFTISSLEPEDIAT |
| | | | | YYCQQGNTLPWTFGCGTKLEIK |
| 135. | BCMA A7 27- | artificial | aa | QVQLVQSGAEVKKPGASVKVSCKASGYTFTNHIIHWV |
| | C4-G7 CC | | | RQAPGQCLEWMGYINPYPGYHAYNEKFQGRATMTSD |
| | (44/100) x | | | TSTSTVYMELSSLRSEDTAVYYCARDGYYRDTDVLDY |
| | I2C0 bispecific | | | WGQGTLVTVSSGGGGSGGGGSGGGGSDIQMTQSPS |
| | molecule | | | SLSASVGDRVTITCQASQDISNYLNWYQQKPGKAPKL |
| | | | | LIYYTSRLHTGVPSRFSGSGSGTDFTFTISSLEPEDIAT |
| | | | | YYCQQGNTLPWTFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYWGQG |
| | | | | TLVTVSSGGGGSGGGGSGGGGSQTWTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVL |
| 136. | BCMA A7 27- | artificial | aa | QVQLVQSGAEVKKPGASVKVSCKASGYTFTNHIIHWV |
| | C4-G7 CC | | | RQAPGQCLEWMGYINPYPGYHAYNEKFQGRATMTSD |
| | (44/100) x | | | TSTSTVYMELSSLRSEDTAVYYCARDGYYRDTDVLDY |
| | I2C0-scFc | | | WGQGTLVTVSSGGGGSGGGGSGGGGSDIQMTQSPS |
| | bispecific | | | SLSASVGDRVTITCQASQDISNYLNWYQQKPGKAPKL |
| | molecule HLE | | | LIYYTSRLHTGVPSRFSGSGSGTDFTFTISSLEPEDIAT |
| | | | | YYCQQGNTLPWTFGCGTKVEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYWGQG |
| | | | | TLVTVSSGGGGSGGGGSGGGGSQTVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 137. | PM 76-B10.17 | artificial | aa | DYYMY |
| | CC VH CDR1 | | | |
| 138. | PM 76-B10.17 | artificial | aa | IISDAGYYTYYSDIIKG |
| | CC VH CDR2 | | | |
| 139. | PM 76-B10.17 | artificial | aa | GFPLLRHGAMDY |
| | CC VH CDR3 | | | |
| 140. | PM 76-B10.17 | artificial | aa | KASQNVDANVA |
| | CC VL CDR1 | | | |
| 141. | PM 76-B10.17 | artificial | aa | SASYVYW |
| | CC VL CDR2 | | | |
| 142. | PM 76-B10.17 | artificial | aa | QQYDQQLIT |
| | CC VL CDR3 | | | |
| 143. | PM 76-B10.17 | artificial | aa | QVQLVESGGGLVKPGESLRLSCAASGFTFSDYYMYW |
| | CC VH | | | VRQAPGKCLEWVAIISDAGYYTYYSDIIKGRFTISRDNA |
| | | | | KNSLYLQMNSLKAEDTAVYYCARGFPLLRHGAMDYW |
| | | | | GQGTLVTVSS |
| 144. | PM 76-B10.17 | artificial | aa | DIQMTQSPSSLSASVGDRVTITCKASQNVDANVAWYQ |
| | CC VL | | | QKPGQAPKSLIYSASYVYWDVPSRFSGSASGTDFTLTI |
| | | | | SSVQSEDFATYYCQQYDQQLITFGCGTKLEIK |
| 145. | PM 76-B10.17 | artificial | aa | QVQLVESGGGLVKPGESLRLSCAASGFTFSDYYMYW |
| | CC scFv | | | VRQAPGKCLEWVAIISDAGYYTYYSDIIKGRFTISRDNA |
| | | | | KNSLYLQMNSLKAEDTAVYYCARGFPLLRHGAMDYW |
| | | | | GQGTLVTVSSGGGGSGGGGSGGGGSDIQMTQSPSS |
| | | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIK |
| 146. | PM 76-B10.17 | artificial | aa | QVQLVESGGGLVKPGESLRLSCAASGFTFSDYYMYW |
| | CC x I2C0 | | | VRQAPGKCLEWVAIISDAGYYTYYSDIIKGRFTISRDNA |
| | bispecific | | | KNSLYLQMNSLKAEDTAVYYCARGFPLLRHGAMDYW |
| | molecule | | | GQGTLVTVSSGGGGSGGGGSGGGGSDIQMTQSPSS |
| | | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYWGQG |
| | | | | TLVTVSSGGGGSGGGGSGGGGSQTWTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVL |
| 147. | PM 76-B10.17 | artificial | aa | QVQLVESGGGLVKPGESLRLSCAASGFTFSDYYMYW |
| | CC x I2C0- | | | VRQAPGKCLEWVAIISDAGYYTYYSDIIKGRFTISRDNA |
| | scFc bispecific | | | KNSLYLQMNSLKAEDTAVYYCARGFPLLRHGAMDYW |
| | HLE molecule | | | GQGTLVTVSSGGGGSGGGGSGGGGSDIQMTQSPSS |
| | | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYWGQG |
| | | | | TLVTVSSGGGGSGGGGSGGGGSQTWTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 148. | PM 76-B10.17 | artificial | aa | QVQLVESGGGLVKPGESLRLSCAASGFTFSDYYMYW |
| | CC x I2C0- | | | VRQAPGKCLEWVAIISDAGYYTYYSDIIKGRFTISRDNA |
| | scFc_delGK | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYWGQG |
| | | | | TLVTVSSGGGGSGGGGSGGGGSQTWTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 149. | PM 76-B10.17 | artificial | aa | QVQLVESGGGLVKPGESLRLSCAASGFTFSDYYMYW |
| | CC x I2C0 CC | | | VRQAPGKCLEWVAIISDAGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | molecule | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYCGQG |
| | | | | TLVTVSSGGGGSGGGGSGGGGSQTWTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVL |
| 150. | PM 76-B10.17 | artificial | aa | QVQLVESGGGLVKPGESLRLSCAASGFTFSDYYMYW |
| | CC x I2C0 CC | | | VRQAPGKCLEWVAIISDAGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDANVAWYQQKPGQAPKS |
| | | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYCGQG |
| | | | | TLVTVSSGGGGSGGGGSGGGGSQTWTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 151. | PM 76-B10.17 | artificial | aa | QVQLVESGGGLVKPGESLRLSCAASGFTFSDYYMYW |
| | CC x I2C0 CC | | | VRQAPGKCLEWVAIISDAGYYTYYSDIIKGRFTISRDNA |
| | (103/43)- | | | KNSLYLQMNSLKAEDTAVYYCARGFPLLRHGAMDYW |
| | scFc_delGK | | | GQGTLVTVSSGGGGGGGGSGGGGSDIQMTQSPSS |
| | bispecific | | | LSASVGDRVTITCKASQNVDANVAWYQQKPGQAPKS |
| | HLE molecule | | | LIYSASYVYWDVPSRFSGSASGTDFTLTISSVQSEDFA |
| | | | | TYYCQQYDQQLITFGCGTKLEIKSGGGGSEVQLVESG |
| | | | | GGLVQPGGSLKLSCAASGFTFNKYAMNWVRQAPGKG |
| | | | | LEWVARIRSKYNNYATYYADSVKDRFTISRDDSKNTAY |
| | | | | LQMNNLKTEDTAVYYCVRHGNFGNSYISYWAYCGQG |
| | | | | TLVTVSSGGGGSGGGGSGGGGSQTWTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 152. | PM 76-B10.11 | artificial | aa | DYYMY |
| | CC VH CDR1 | | | |
| 153. | PM 76-B10.11 | artificial | aa | IISDGGYYTYYSDIIKG |
| | CC VH CDR2 | | | |
| 154. | PM 76-B10.11 | artificial | aa | GFPLLRHGAMDY |
| | CC VH CDR3 | | | |
| 155. | PM 76-B10.11 | artificial | aa | KASQNVDTNVA |
| | CC VL CDR1 | | | |
| 156. | PM 76-B10.11 | artificial | aa | SASYVYW |
| | CC VL CDR2 | | | |
| 157. | PM 76-B10.11 | artificial | aa | QQYDQQLIT |
| | CC VL CDR3 | | | |
| 158. | PM 76-B10.11 | artificial | aa | QVQLVESGGGLVKPGESLRLSCAASGFTFSDYYMYW |
| | CC VH | | | VRQAPGKGLEWVAIISDGGYYTYYSDIIKGRFTISRDNA |
| | | | | KNSLYLQMNSLKAEDTAVYYCARGFPLLRHGAMDYW |
| | | | | GQGTLVTVSS |
| 159. | PM 76-B10.11 | artificial | aa | DIQMTQSPSSLSASVGDRVTITCKASQNVDTNVAWYQ |
| | CC VL | | | QKPGQAPKSLIYSASYVYWDVPSRFSGSASGTDFTLTI |
| | | | | SSVQSEDFATYYCQQYDQQLITFGGGTKLEIK |
| 160. | PM 76-B10.11 | artificial | aa | QVQLVESGGGLVKPGESLRLSCAASGFTFSDYYMYW |
| | CC scFv | | | VRQAPGKGLEWVAIISDGGYYTYYSDIIKGRFTISRDNA |
| | | | | KNSLYLQMNSLKAEDTAVYYCARGFPLLRHGAMDYW |
| | | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIK |
| 161. | PM 76-B10.11 | artificial | aa | QVQLVESGGGLVKPGESLRLSCAASGFTFSDYYMYW |
| | CC x I2C0 | | | VRQAPGKGLEWVAIISDGGYYTYYSDIIKGRFTISRDNA |
| | bispecific | | | KNSLYLQMNSLKAEDTAVYYCARGFPLLRHGAMDYW |
| | molecule | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGSGGGGSGGGGSQTVVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVL |
| 162. | PM 76-B10.11 | artificial | aa | QVQLVESGGGLVKPGESLRLSCAASGFTFSDYYMYW |
| | CC x I2C0- | | | VRQAPGKGLEWVAIISDGGYYTYYSDIIKGRFTISRDNA |
| | scFc bispecific | | | KNSLYLQMNSLKAEDTAVYYCARGFPLLRHGAMDYW |
| | HLE molecule | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGSGGGGSGGGGSQTVVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 163. | PM 76-B10.11 | artificial | aa | QVQLVESGGGLVKPGESLRLSCAASGFTFSDYYMYW |
| | CC x I2C0- | | | VRQAPGKGLEWVAIISDGGYYTYYSDIIKGRFTISRDNA |
| | scFc_delGK | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGSGGGGSGGGGSQTWTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 164. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTVVTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVL |
| 165. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTVVTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 166. | PM 76-B10.11 | artificial | aa | QVQLVESGGGLVKPGESLRLSCAASGFTFSDYYMYW |
| | CC x I2C0 CC | | | VRQAPGKGLEWVAIISDGGYYTYYSDIIKGRFTISRDNA |
| | (103/43)- | | | KNSLYLQMNSLKAEDTAVYYCARGFPLLRHGAMDYW |
| | scFc_delGK | | | GQGTLVTVSSGGGGSGGGGSGGGGSDIQMTQSPSS |
| | bispecific | | | LSASVGDRVTITCKASQNVDTNVAWYQQKPGQAPKSL |
| | HLE molecule | | | IYSASYVYWDVPSRFSGSASGTDFTLTISSVQSEDFAT |
| | | | | YYCQQYDQQLITFGGGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTVVTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 167. | PM 76-B10.11 | artificial | aa | DYYMY |
| | CC x I2C0- | | | |
| | scFc VH CDR1 | | | |
| 168. | PM 76-B10.11 | artificial | aa | IISDGGYYTYYSDIIKG |
| | CC x I2C0- | | | |
| | scFc VH CDR2 | | | |
| 169. | PM 76-B10.11 | artificial | aa | GFPLLRHGAMDY |
| | CC x I2C0- | | | |
| | scFc VH CDR3 | | | |
| 170 | PM 76-B10.11 | artificial | aa | KASQNVDTNVA |
| | CC x I2C0- | | | |
| | scFc VL CDR1 | | | |
| 171 | PM 76-B10.11 | artificial | aa | SASYVYW |
| | CC x I2C0- | | | |
| | scFc VL CDR2 | | | |
| 172. | PM 76-B10.11 | artificial | aa | QQYDQQLIT |
| | CC x I2C0- | | | |
| | scFc VL CDR3 | | | |
| 173. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDNA |
| | scFc VH | | | KNSLYLQMNSLKAEDTAVYYCARGFPLLRHGAMDYW |
| | | | | GQGTLVTVSS |
| 174. | PM 76-B10.11 | artificial | aa | DIQMTQSPSSLSASVGDRVTITCKASQNVDTNVAWYQ |
| | CC x I2C0- | | | QKPGQAPKSLIYSASYVYWDVPSRFSGSASGTDFTLTI |
| | scFc VL | | | SSVQSEDFATYYCQQYDQQLITFGCGTKLEIK |
| 175. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDNA |
| | scFc scFv | | | KNSLYLQMNSLKAEDTAVYYCARGFPLLRHGAMDYW |
| | | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIK |
| 176. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDNA |
| | scFc bispecific | | | KNSLYLQMNSLKAEDTAVYYCARGFPLLRHGAMDYW |
| | molecule | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGSGGGGSGGGGSQTVVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVL |
| 177. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDNA |
| | scFc bispecific | | | KNSLYLQMNSLKAEDTAVYYCARGFPLLRHGAMDYW |
| | HLE molecule | | | GQGTLVTVSSGGGGSGGGGSGGGGSDIQMTQSPSS |
| | | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGSGGGGSGGGGSQTVVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 178. | PM 76-B10.11 | artificial | aa | QVQLVESGGGLVKPGESLRLSCAASGFTFSDYYMYW |
| | CC x I2C0- | | | VRQAPGKCLEWVAIISDGGYYTYYSDIIKGRFTISRDNA |
| | scFc_delGK | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYWGQGT |
| | | | | LVTVSSGGGGGGGGSGGGGSQTVVTQEPSLTVSP |
| | | | | GGTVTLTCGSSTGAVTSGNYPNWVQQKPGQAPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 179. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTVVTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVL |
| 180. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDNA |
| | (103/43)-scFc | | | KNSLYLQMNSLKAEDTAVYYCARGFPLLRHGAMDYW |
| | bispecific | | | GQGTLVTVSSGGGGSGGGGSGGGGSDIQMTQSPSS |
| | HLE molecule | | | LSASVGDRVTITCKASQNVDTNVAWYQQKPGQAPKSL |
| | | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTVVTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGKGGGG |
| | | | | SGGGGSGGGGSGGGGSGGGGSGGGGSDKTHTCPP |
| | | | | CPAPELLGGPSVFLFPPKPKDTLMISRTPEVTCVVVDV |
| | | | | SHEDPEVKFNWYVDGVEVHNAKTKPCEEQYGSTYRC |
| | | | | VSVLTVLHQDWLNGKEYKCKVSNKALPAPIEKTISKAK |
| | | | | GQPREPQVYTLPPSREEMTKNQVSLTCLVKGFYPSDI |
| | | | | AVEWESNGQPENNYKTTPPVLDSDGSFFLYSKLTVDK |
| | | | | SRWQQGNVFSCSVMHEALHNHYTQKSLSLSPGK |
| 181. | PM 76-B10.11 | artificial | aa | QVQLVESGGGLVKPGESLRLSCAASGFTFSDYYMYW |
| | CC x I2C0 CC | | | VRQAPGKCLEWVAIISDGGYYTYYSDIIKGRFTISRDNA |
| | (103/43)- | | | KNSLYLQMNSLKAEDTAVYYCARGFPLLRHGAMDYW |
| | scFc_delGK | | | GQGTLVTVSSGGGGSGGGGSGGGGSDIQMTQSPSS |
| | bispecific | | | LSASVGDRVTITCKASQNVDTNVAWYQQKPGQAPKSL |
| | HLE molecule | | | IYSASYVYWDVPSRFSGSASGTDFTLTISSVQSEDFAT |
| | | | | YYCQQYDQQLITFGCGTKLEIKSGGGGSEVQLVESGG |
| | | | | GLVQPGGSLKLSCAASGFTFNKYAMNWVRQAPGKGL |
| | | | | EWVARIRSKYNNYATYYADSVKDRFTISRDDSKNTAYL |
| | | | | QMNNLKTEDTAVYYCVRHGNFGNSYISYWAYCGQGT |
| | | | | LVTVSSGGGGSGGGGSGGGGSQTWTQEPSLTVSP |
| | | | | GGTVTLTCGSSTGAVTSGNYPNWVQQKPGQCPRGLI |
| | | | | GGTKFLAPGTPARFSGSLLGGKAALTLSGVQPEDEAE |
| | | | | YYCVLWYSNRWVFGGGTKLTVLGGGGDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGGGGSG |
| | | | | GGGSGGGGSGGGGSGGGGSGGGGSDKTHTCPPCP |
| | | | | APELLGGPSVFLFPPKPKDTLMISRTPEVTCVVVDVSH |
| | | | | EDPEVKFNWYVDGVEVHNAKTKPCEEQYGSTYRCVS |
| | | | | VLTVLHQDWLNGKEYKCKVSNKALPAPIEKTISKAKGQ |
| | | | | PREPQVYTLPPSREEMTKNQVSLTCLVKGFYPSDIAVE |
| | | | | WESNGQPENNYKTTPPVLDSDGSFFLYSKLTVDKSR |
| | | | | WQQGNVFSCSVMHEALHNHYTQKSLSLSPGK |
| 182. | IgG1 hinge | artificial | aa | DKTHTCPPCP |
| 183. | IgG2 subtype | artificial | aa | ERKCCVECPPCP |
| | hinge | | | |
| 184. | IgG3 subtype | artificial | aa | ELKTPLDTTHTCPRCP |
| | hinge | | | |
| 185. | IgG3 subtype | artificial | aa | ELKTPLGDTTHTCPRCP |
| | hinge | | | |
| 186. | IgG4 subtype | artificial | aa | ESKYGPPCPSCP |
| | hinge | | | |
| 187. | G4S linker | artificial | aa | GGGGS |
| 188. | (G4S)2 linker | artificial | aa | GGGGSGGGGS |
| 189. | (G4S)3 linker | artificial | aa | GGGGSGGGGSGGGGS |
| 190. | (G4S)4 linker | artificial | aa | GGGGSGGGGSGGGGSGGGGS |
| 191. | (G4S)5 linker | artificial | aa | GGGGSGGGGSGGGGSGGGGSGGGGS |
| 192. | (G4S)6 linker | artificial | aa | GGGGSGGGGSGGGGSGGGGSGGGGSGGGGS |
| 193. | (G4S)7 linker | artificial | aa | GGGGSGGGGSGGGGSGGGGSGGGGSGGGGSGGGGS |
| 194. | (G4S)8 linker | artificial | aa | GGGGSGGGGSGGGGSGGGGSGGGGSGGGGSGGGGSGGGGS |
| 195. | Peptide linker | artificial | aa | PGGGGS |
| 196. | Peptide linker | artificial | aa | PGGDGS |
| 197. | Peptide linker | artificial | aa | SGGGGS |
| 198. | Peptide linker | artificial | aa | GGGG |
| 199. | hexa-histidine | artificial | aa | HHHHHH |
| | tag | | | |
| 200. | CD3e binder | artificial | aa | QTVVTQEPSLTVSPGGTVTLTCGSSTGAVTSGNYPN |
| | VL | | | WVQQKPGQAPRGLIGGTKFLAPGTPARFSGSLLGGK |
| | | | | AALTLSGVQPEDEAEYYCVLWYSNRWVFGGGTKLTV |
| | | | | L |
| 201. | CD3e binder | artificial | aa | EVQLVESGGGLVQPGGSLRLSCAASGFTFNSYAMNW |
| | VH | | | VRQAPGKGLEWVARIRSKYNNYATYYADSVKGRFTIS |
| | | | | RDDSKNTAYLQMNSLKTEDTAVYYCVRHGNFGNSYV |
| | | | | SWWAYWGQGTLVTVSS |
| 202. | CD3e binder | artificial | aa | EVQLVESGGGLVQPGGSLRLSCAASGFTFNSYAMNW |
| | scFv | | | VRQAPGKGLEWVARIRSKYNNYATYYADSVKGRFTIS |
| | | | | RDDSKNTAYLQMNSLKTEDTAVYYCVRHGNFGNSYV |
| | | | | SWWAYWGQGTLVTVSSGGGGSGGGGSGGGGSQTV |
| | | | | VTQEPSLTVSPGGTVTLTCGSSTGAVTSGNYPNWVQ |
| | | | | QKPGQAPRGLIGGTKFLAPGTPARFSGSLLGGKAALT |
| | | | | LSGVQPEDEAEYYCVLWYSNRWVFGGGTKLTVL |
| 203. | MU 8-B7 CC x | artificial | aa | GYYWS |
| | I2C0-scFc VH | | | |
| | CDR1 | | | |
| 204. | MU 8-B7 CC x | artificial | aa | DIDASGSTKYNPSLKS |
| | I2C0-scFc VH | | | |
| | CDR2 | | | |
| 205. | MU 8-B7 CC x | artificial | aa | KKYSTVWSYFDN |
| | I2C0-scFc VH | | | |
| | CDR3 | | | |
| 206. | MU 8-B7 CC x | artificial | aa | SGDKLGDKYAS |
| | I2C0-scFc VL | | | |
| | CDR1 | | | |
| 207 | MU 8-B7 CC x | artificial | aa | QDRKRPS |
| | I2C0-scFc VL | | | |
| | CDR2 | | | |
| 208. | MU 8-B7 CC x | artificial | aa | QAWGSSTAV |
| | I2C0-scFc VL | | | |
| | CDR3 | | | |
| 209 | MU 8-B7 CC x | artificial | aa | QVQLQQWGAGLLKPSETLSLTCAVYGGSFSGYYWSWIRQ |
| | I2C0-scFc VH | | | PPGKCLEWIGDIDASGSTKYNPSLKSRVTISLDTSKNQFSLK |
| | | | | LNSVTAADTAVYFCARKKYSTVWSYFDNWGQGTLVTVSS |
| 210. | MU 8-B7 CC x | artificial | aa | SYELTQPSSVSVPPGQTASITCSGDKLGDKYASWYQQKPG |
| | I2C0-scFc VL | | | QSPVLVIYQDRKRPSGVPERFSGSNSGNTATLTISGTQAMD |
| | | | | EADYYCQAWGSSTAVFGCGTKLTVL |
| 211. | MU 8-B7 CC x | artificial | aa | QVQLQQWGAGLLKPSETLSLTCAVYGGSFSGYYWSWIRQ |
| | I2C0-scFc scFv | | | PPGKCLEWIGDIDASGSTKYNPSLKSRVTISLDTSKNQFSLK |
| | | | | LNSVTAADTAVYFCARKKYSTVWSYFDNWGQGTLVTVSSG |
| | | | | GGGSGGGGSGGGGSSYELTQPSSVSVPPGQTASITCSGD |
| | | | | KLGDKYASWYQQKPGQSPVLVIYQDRKRPSGVPERFSGSN |
| | | | | SGNTATLTISGTQAMDEADYYCQAWGSSTAVFGCGTKLTV |
| | | | | L |
| 212. | MU 8-B7 CC x | artificial | aa | QVQLQQWGAGLLKPSETLSLTCAVYGGSFSGYYWSWIRQ |
| | I2C0-scFc | | | PPGKCLEWIGDIDASGSTKYNPSLKSRVTISLDTSKNQFSLK |
| | Bispecific | | | LNSVTAADTAVYFCARKKYSTVWSYFDNWGQGTLVTVSSG |
| | molecule | | | GGGSGGGGSGGGGSSYELTQPSSVSVPPGQTASITCSGD |
| | | | | KLGDKYASWYQQKPGQSPVLVIYQDRKRPSGVPERFSGSN |
| | | | | SGNTATLTISGTQAMDEADYYCQAWGSSTAVFGCGTKLTV |
| | | | | LSGGGGSEVQLVESGGGLVQPGGSLKLSCAASGFTFNKYA |
| | | | | MNWVRQAPGKGLEWVARIRSKYNNYATYYADSVKDRFTIS |
| | | | | RDDSKNTAYLQMNNLKTEDTAVYYCVRHGNFGNSYISYWA |
| | | | | YWGQGTLVTVSSGGGGSGGGGSGGGGSQTVVTQEPSLT |
| | | | | VSPGGTVTLTCGSSTGAVTSGNYPNWVQQKPGQAPRGLIG |
| | | | | GTKFLAPGTPARFSGSLLGGKAALTLSGVQPEDEAEYYCVL |
| | | | | WYSNRWVFGGGTKLTVL |
| 213. | MU 8-B7 CC x | Artificial | aa | QVQLQQWGAGLLKPSETLSLTCAVYGGSFSGYYWSWIRQ |
| | I2C0-scFc | | | PPGKCLEWIGDIDASGSTKYNPSLKSRVTISLDTSKNQFSLK |
| | Bispecific HLE | | | LNSVTAADTAVYFCARKKYSTVWSYFDNWGQGTLVTVSSG |
| | molecule | | | GGGSGGGGSGGGGSSYELTQPSSVSVPPGQTASITCSGD |
| | | | | KLGDKYASWYQQKPGQSPVLVIYQDRKRPSGVPERFSGSN |
| | | | | SGNTATLTISGTQAMDEADYYCQAWGSSTAVFGCGTKLTV |
| | | | | LSGGGGSEVQLVESGGGLVQPGGSLKLSCAASGFTFNKYA |
| | | | | MNWVRQAPGKGLEWVARIRSKYNNYATYYADSVKDRFTIS |
| | | | | RDDSKNTAYLQMNNLKTEDTAVYYCVRHGNFGNSYISYWA |
| | | | | YWGQGTLVTVSSGGGGSGGGGSGGGGSQTVVTQEPSLT |
| | | | | VSPGGTVTLTCGSSTGAVTSGNYPNWVQQKPGQAPRGLIG |
| | | | | GTKFLAPGTPARFSGSLLGGKAALTLSGVQPEDEAEYYCVL |
| | | | | WYSNRWVFGGGTKLTVLGGGGDKTHTCPPCPAPELLGGP |
| | | | | SVFLFPPKPKDTLMISRTPEVTCVVVDVSHEDPEVKFNWYV |
| | | | | DGVEVHNAKTKPCEEQYGSTYRCVSVLTVLHQDWLNGKEY |
| | | | | KCKVSNKALPAPIEKTISKAKGQPREPQVYTLPPSREEMTK |
| | | | | NQVSLTCLVKGFYPSDIAVEWESNGQPENNYKTTPPVLDS |
| | | | | DGSFFLYSKLTVDKSRWQQGNVFSCSVMHEALHNHYTQK |
| | | | | SLSLSPGKGGGGSGGGGSGGGGSGGGGSGGGGSGGGG |
| | | | | SDKTHTCPPCPAPELLGGPSVFLFPPKPKDTLMISRTPEVT |
| | | | | CVVVDVSHEDPEVKFNWYVDGVEVHNAKTKPCEEQYGST |
| | | | | YRCVSVLTVLHQDWLNGKEYKCKVSNKALPAPIEKTISKAK |
| | | | | GQPREPQVYTLPPSREEMTKNQVSLTCLVKGFYPSDIAVE |
| | | | | WESNGQPENNYKTTPPVLDSDGSFFLYSKLTVDKSRWQQ |
| | | | | GNVFSCSVMHEALHNHYTQKSLSLSPGK |
| 214. | CL-1 VH CDR1 | Artificial | aa | GYYMH |
| 215. | CL-1 VH CDR2 | Artificia | aa | WINPNSGGTKYAQKFQG |
| 216. | CL-1 VH CDR3 | Artificial | aa | DRITVAGTYYYYGMDV |
| 217. | CL-1 VL CDR1 | Artificial | aa | RASQGVNNWLA |
| 218. | CL-1 VL CDR2 | Artificial | aa | TASSLQS |
| 219. | CL-1 VL CDR3 | Artificial | aa | QQANSFPIT |
| 220. | CL-1 VH | Artificial | aa | QVQLVQSGAEVKKPGASVKVSCKASGYTFTGYYMHWVRQ |
| | | | | APGQCLEWMGWINPNSGGTKYAQKFQGRVTMTRDTSIST |
| | | | | AYMELSRLRSDDTAVYYCARDRITVAGTYYYYGMDVWGQ |
| | | | | GTTVTVSS |
| 221. | CL-1 VL | Artificia | aa | DIQMTQSPSSVSASVGDRVTITCRASQGVNNWLAWYQQKP |
| | | | | GKAPKLLIYTASSLQSGVPSRFSGSGSGTDFTLTIRSLQPED |
| | | | | FATYYCQQANSFPITFGCGTRLEIK |
| 222. | CL-1 scFv | Artificial | aa | QVQLVQSGAEVKKPGASVKVSCKASGYTFTGYYMHWVRQ |
| | | | | APGQCLEWMGWINPNSGGTKYAQKFQGRVTMTRDTSIST |
| | | | | AYMELSRLRSDDTAVYYCARDRITVAGTYYYYGMDVWGQ |
| | | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIK |
| 223. | CL-1 x I2C | | | |
| | bispecific | | | |
| 224 | molecule | Artificial | aa | QVQLVQSGAEVKKPGASVKVSCKASGYTFTGYYMHWVRQ |
| | | | | APGQCLEWMGWINPNSGGTKYAQKFQGRVTMTRDTSIST |
| | | | | AYMELSRLRSDDTAVYYCARDRITVAGTYYYYGMDVWGQ |
| | | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVL |
| 225. | CL-1 x I2C-6His | Artificial | aa | QVQLVQSGAEVKKPGASVKVSCKASGYTFTGYYMHWVRQ |
| | bispecific | | | APGQCLEWMGWINPNSGGTKYAQKFQGRVTMTRDTSIST |
| | molecule-his | | | AYMELSRLRSDDTAVYYCARDRITVAGTYYYYGMDVWGQ |
| | tag | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGSGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVLHHHHHH |
| 126. | CL-1 x I2C-scFc | artificial | aa | QVQLVQSGAEVKKPGASVKVSCKASGYTFTGYYMHWVRQ |
| | bispecific scFc | | | APGQCLEWMGWINPNSGGTKYAQKFQGRVTMTRDTSIST |
| | molecule | | | AYMELSRLRSDDTAVYYCARDRITVAGTYYYYGMDVWGQ |
| | | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGSGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVLGGGGDKTHTCPP |
| | | | | CPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGKGGGGSGGGGSGGGGSGGGGSG |
| | | | | GGGSGGGGSDKTHTCPPCPAPELLGGPSVFLFPPKPKDTL |
| | | | | MISRTPEVTCVVVDVSHEDPEVKFNWYVDGVEVHNAKTKP |
| | | | | CEEQYGSTYRCVSVLTVLHQDWLNGKEYKCKVSNKALPAP |
| | | | | IEKTISKAKGQPREPQVYTLPPSREEMTKNQVSLTCLVKGF |
| | | | | YPSDIAVEWESNGQPENNYKTTPPVLDSDGSFFLYSKLTVD |
| | | | | KSRWQQGNVFSCSVMHEALHNHYTQKSLSLSPGK |
| 227. | CL-2 VH CDR1 | Artificial | aa | GYYMH |
| 228. | CL-2 VH CDR2 | Artificial | aa | WINPNSGGTKYAQKFQG |
| 229. | CL-2 VH CDR3 | Artificial | aa | DRITVAGTYYYYGMDV |
| 230. | CL-2 VL CDR1 | Artificial | aa | RASQGVNNWLA |
| 231. | CL-2VL CDR2 | Artificial | aa | TASSLQS |
| 232. | CL-2 VL CDR3 | Artificial | aa | QQANSFPIT |
| 233. | CL-2 VH | Artificial | aa | QVQMVQSGAEVKKHGASVKVSCKASGYTFTGYYMHWVR |
| | | | | QAPGQCLEWMGWINPNSGGTKYAQKFQGRVTMTRDTSIS |
| | | | | TAYMELSRLRSDDTAVYYCARDRITVAGTYYYYGMDVWGQ |
| | | | | GTTVTVSS |
| 234. | CL-2 VL | Artificial | aa | DIQMTQSPSSVSASVGDRVTITCRASQGVNNWLAWYQQKP |
| | | | | GKAPKLLIYTASSLQSGVPSRFSGSGSGTDFTLTIRSLQPED |
| | | | | FATYYCQQANSFPITFGCGTRLEIK |
| 235. | CL-2 scFv | Artificial | aa | QVQMVQSGAEVKKHGASVKVSCKASGYTFTGYYMHWVR |
| | | | | QAPGQCLEWMGWINPNSGGTKYAQKFQGRVTMTRDTSIS |
| | | | | TAYMELSRLRSDDTAVYYCARDRITVAGTYYYYGMDVWGQ |
| | | | | GTTVTVSSGGGGGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIK |
| 236. | CL-2 x I2C | Artificial | aa | QVQMVQSGAEVKKHGASVKVSCKASGYTFTGYYMHWVR |
| | bispecific | | | QAPGQCLEWMGWINPNSGGTKYAQKFQGRVTMTRDTSIS |
| | molecule | | | TAYMELSRLRSDDTAVYYCARDRITVAGTYYYYGMDVWGQ |
| | | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGSGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVL |
| 237. | CL-2 x I2C-6His | Artificial | aa | QVQMVQSGAEVKKHGASVKVSCKASGYTFTGYYMHWVR |
| | bispecific | | | QAPGQCLEWMGWINPNSGGTKYAQKFQGRVTMTRDTSIS |
| | molecule- his | | | TAYMELSRLRSDDTAVYYCARDRITVAGTYYYYGMDVWGQ |
| | tag | | | GTTVTVSSGGGGSGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGSGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVLHHHHHH |
| 238. | CL-2 x I2C-scFc | Artificial | aa | QVQMVQSGAEVKKHGASVKVSCKASGYTFTGYYMHWVR |
| | bispecific scFc | | | QAPGQCLEWMGWINPNSGGTKYAQKFQGRVTMTRDTSIS |
| | molecule | | | TAYMELSRLRSDDTAVYYCARDRITVAGTYYYYGMDVWGQ |
| | | | | GTTVTVSSGGGGGGGGSGGGGSDIQMTQSPSSVSASVG |
| | | | | DRVTITCRASQGVNNWLAWYQQKPGKAPKLLIYTASSLQS |
| | | | | GVPSRFSGSGSGTDFTLTIRSLQPEDFATYYCQQANSFPIT |
| | | | | FGCGTRLEIKSGGGGSEVQLVESGGGLVQPGGSLKLSCAA |
| | | | | SGFTFNKYAMNWVRQAPGKGLEWVARIRSKYNNYATYYA |
| | | | | DSVKDRFTISRDDSKNTAYLQMNNLKTEDTAVYYCVRHGN |
| | | | | FGNSYISYWAYWGQGTLVTVSSGGGGSGGGGSGGGGSQ |
| | | | | TVVTQEPSLTVSPGGTVTLTCGSSTGAVTSGNYPNWVQQK |
| | | | | PGQAPRGLIGGTKFLAPGTPARFSGSLLGGKAALTLSGVQP |
| | | | | EDEAEYYCVLWYSNRWVFGGGTKLTVLGGGGDKTHTCPP |
| | | | | CPAPELLGGPSVFLFPPKPKDTLMISRTPEVTCVVVDVSHE |
| | | | | DPEVKFNWYVDGVEVHNAKTKPCEEQYGSTYRCVSVLTVL |
| | | | | HQDWLNGKEYKCKVSNKALPAPIEKTISKAKGQPREPQVYT |
| | | | | LPPSREEMTKNQVSLTCLVKGFYPSDIAVEWESNGQPENN |
| | | | | YKTTPPVLDSDGSFFLYSKLTVDKSRWQQGNVFSCSVMHE |
| | | | | ALHNHYTQKSLSLSPGKGGGGSGGGGSGGGGSGGGGSG |
| | | | | GGGSGGGGSDKTHTCPPCPAPELLGGPSVFLFPPKPKDTL |
| | | | | MISRTPEVTCVVVDVSHEDPEVKFNWYVDGVEVHNAKTKP |
| | | | | CEEQYGSTYRCVSVLTVLHQDWLNGKEYKCKVSNKALPAP |
| | | | | IEKTISKAKGQPREPQVYTLPPSREEMTKNQVSLTCLVKGF |
| | | | | YPSDIAVEWESNGQPENNYKTTPPVLDSDGSFFLYSKLTVD |
| | | | | KSRWQQGNVFSCSVMHEALHNHYTQKSLSLSPGK |
| 239. | CD70 HCDR1 | Artificial | aa | TYAMS |
| 240. | CD70 HCDR2 | Artificial | aa | AISGSGGRTFYAESVEG |
| 241. | CD70 HCDR3 | Artificial | aa | HDYSNYPYFDY |
| 242. | CD70 LCDR1 | Artificial | aa | RASQSVRSTYLA |
| 243. | CD70 LCDR2 | Artificial | aa | GASSRAT |
| 244. | CD70 LCDR3 | Artificial | aa | QQYGDLPFT |
| 245. | CD70 VH | Artificial | aa | EVQLLESGGGMVQPGGSLRLSCAASGFTFSTYAMSWVRQ |
| | | | | APGKCLEWVSAISGSGGRTFYAESVEGRFTISRDNSKNTLY |
| | | | | LQMNSLRAEDTAVYYCAKHDYSNYPYFDYWGQGTLVTVSS |
| 246. | CD70 VL | Artificial | aa | EIVLTQSPGTLSLSPGERATLSCRASQSVRSTYLAWYQQKP |
| | | | | GQAPRLLIYGASSRATGIPDRFSGSGSGTDFTLTISRLEPED |
| | | | | FAVYSCQQYGDLPFTFGCGTKLEIK |
| 247. | CD70 VHVL | Artificial | aa | EVQLLESGGGMVQPGGSLRLSCAASGFTFSTYAMSWVRQ |
| | | | | APGKCLEWVSAISGSGGRTFYAESVEGRFTISRDNSKNTLY |
| | | | | LQMNSLRAEDTAVYYCAKHDYSNYPYFDYWGQGTLVTVSS |
| | | | | GGGGSGGGGSGGGGSEIVLTQSPGTLSLSPGERATLSCRA |
| | | | | SQSVRSTYLAWYQQKPGQAPRLLIYGASSRATGIPDRFSGS |
| | | | | GSGTDFTLTISRLEPEDFAVYSCQQYGDLPFTFGCGTKLEI |
| 248. | CD70 x I2C | Artificial | aa | EVQLLESGGGMVQPGGSLRLSCAASGFTFSTYAMSWVRQ |
| | | | | APGKCLEWVSAISGSGGRTFYAESVEGRFTISRDNSKNTLY |
| | | | | LQMNSLRAEDTAVYYCAKHDYSNYPYFDYWGQGTLVTVSS |
| | | | | GGGGSGGGGSGGGGSEIVLTQSPGTLSLSPGERATLSCRA |
| | | | | SQSVRSTYLAWYQQKPGQAPRLLIYGASSRATGIPDRFSGS |
| | | | | GSGTDFTLTISRLEPEDFAVYSCQQYGDLPFTFGCGTKLEIK |
| | | | | SGGGGSEVQLVESGGGLVQPGGSLKLSCAASGFTFNKYA |
| | | | | MNWVRQAPGKGLEWVARIRSKYNNYATYYADSVKDRFTIS |
| | | | | RDDSKNTAYLQMNNLKTEDTAVYYCVRHGNFGNSYISYWA |
| | | | | YWGQGTLVTVSSGGGGSGGGGSGGGGSQTVVTQEPSLT |
| | | | | VSPGGTVTLTCGSSTGAVTSGNYPNWVQQKPGQAPRGLIG |
| | | | | GTKFLAPGTPARFSGSLLGGKAALTLSGVQPEDEAEYYCVL |
| | | | | WYSNRWVFGGGTKLTV |
| 249. | Fc monomer-1 | Artificial | aa | DKTHTCPPCPAPELLGGPSVFLFPPKPKDTLMISRTPE |
| | +c/−g | | | VTCVVVDVSHEDPEVKFNWYVDGVEVHNAKTKPCEE |
| | | | | QYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGK |
| 250. | Fc monomer-2 | Artificial | aa | DKTHTCPPCPAPELLGGPSVFLFPPKPKDTLMISRTPE |
| | +c/−g/delGK | | | VTCVVVDVSHEDPEVKFNWYVDGVEVHNAKTKPCEE |
| | | | | QYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSP |
| 251. | Fc monomer-3 | Artificial | aa | DKTHTCPPCPAPELLGGPSVFLFPPKPKDTLMISRTPE |
| | −c/+g | | | VTCVVVDVSHEDPEVKFNWYVDGVEVHNAKTKPREE |
| | | | | QYNSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGK |
| 252. | Fc monomer-4 | Artificial | aa | DKTHTCPPCPAPELLGGPSVFLFPPKPKDTLMISRTPE |
| | −c/+g/delGK | | | VTCVVVDVSHEDPEVKFNWYVDGVEVHNAKTKPREE |
| | | | | QYNSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSP |
| 253. | Fc monomer-5 | Artificial | aa | DKTHTCPPCPAPELLGGPSVFLFPPKPKDTLMISRTPE |
| | −c/−g | | | VTCVVVDVSHEDPEVKFNWYVDGVEVHNAKTKPREE |
| | | | | QYGSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGK |
| 254. | Fc monomer-6 | Artificial | aa | DKTHTCPPCPAPELLGGPSVFLFPPKPKDTLMISRTPE |
| | −c/−g/deIGK | | | VTCVVVDVSHEDPEVKFNWYVDGVEVHNAKTKPREE |
| | | | | QYGSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSP |
| 255. | Fc monomer-7 | Artificial | aa | DKTHTCPPCPAPELLGGPSVFLFPPKPKDTLMISRTPE |
| | +c/+g | | | VTCVVVDVSHEDPEVKFNWYVDGVEVHNAKTKPCEE |
| | | | | QYNSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGK |
| 256. | Fc monomer-8 | Artificial | aa | DKTHTCPPCPAPELLGGPSVFLFPPKPKDTLMISRTPE |
| | +c/+g/delGK | | | VTCVVVDVSHEDPEVKFNWYVDGVEVHNAKTKPCEE |
| | | | | QYNSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSP |
| 257. | scFc-1 | Artificial | aa | DKTHTCPPCPAPELLGGPSVFLFPPKPKDTLMISRTPE |
| | | | | VTCVVVDVSHEDPEVKFNWYVDGVEVHNAKTKPCEE |
| | | | | QYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGKGGGGSGGGGSGGGGSGGGGSGGGGSGG |
| | | | | GGSDKTHTCPPCPAPELLGGPSVFLFPPKPKDTLMIS |
| | | | | RTPEVTCVVVDVSHEDPEVKFNWYVDGVEVHNAKTK |
| | | | | PCEEQYGSTYRCVSVLTVLHQDWLNGKEYKCKVSNK |
| | | | | ALPAPIEKTISKAKGQPREPQVYTLPPSREEMTKNQVS |
| | | | | LTCLVKGFYPSDIAVEWESNGQPENNYKTTPPVLDSD |
| | | | | GSFFLYSKLTVDKSRWQQGNVFSCSVMHEALHNHYT |
| | | | | QKSLSLSPGK |
| 258. | scFc-2 | Artificial | aa | DKTHTCPPCPAPELLGGPSVFLFPPKPKDTLMISRTPE |
| | | | | VTCVVVDVSHEDPEVKFNWYVDGVEVHNAKTKPCEE |
| | | | | QYGSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGGGGGGGGSGGGGSGGGGSGGGGSGGGG |
| | | | | SDKTHTCPPCPAPELLGGPSVFLFPPKPKDTLMISRTP |
| | | | | EVTCVWVDVSHEDPEVKFNWYVDGVEVHNAKTKPCE |
| | | | | EQYGSTYRCVSVLTVLHQDWLNGKEYKCKVSNKALP |
| | | | | APIEKTISKAKGQPREPQVYTLPPSREEMTKNQVSLTC |
| | | | | LVKGFYPSDIAVEWESNGQPENNYKTTPPVLDSDGSF |
| | | | | FLYSKLTVDKSRWQQGNVFSCSVMHEALHNHYTQKS |
| | | | | LSLSP |
| 259. | scFc-3 | Artificial | aa | DKTHTCPPCPAPELLGGPSVFLFPPKPKDTLMISRTPE |
| | | | | VTCVVVDVSHEDPEVKFNWYVDGVEVHNAKTKPREE |
| | | | | QYNSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGKGGGGSGGGGSGGGGSGGGGSGGGGSGG |
| | | | | GGSDKTHTCPPCPAPELLGGPSVFLFPPKPKDTLMIS |
| | | | | RTPEVTCVVVDVSHEDPEVKFNWYVDGVEVHNAKTK |
| | | | | PREEQYNSTYRVVSVLTVLHQDWLNGKEYKCKVSNK |
| | | | | ALPAPIEKTISKAKGQPREPQVYTLPPSREEMTKNQVS |
| | | | | LTCLVKGFYPSDIAVEWESNGQPENNYKTTPPVLDSD |
| | | | | GSFFLYSKLTVDKSRWQQGNVFSCSVMHEALHNHYT |
| | | | | QKSLSLSPGK |
| 260. | scFc-4 | Artificial | aa | DKTHTCPPCPAPELLGGPSVFLFPPKPKDTLMISRTPE |
| | | | | VTCVVVDVSHEDPEVKFNWYVDGVEVHNAKTKPREE |
| | | | | QYNSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGGGGSGGGGSGGGGSGGGGSGGGGSGGGG |
| | | | | SDKTHTCPPCPAPELLGGPSVFLFPPKPKDTLMISRTP |
| | | | | EVTCVWVDVSHEDPEVKFNWYVDGVEVHNAKTKPRE |
| | | | | EQYNSTYRVVSVLTVLHQDWLNGKEYKCKVSNKALP |
| | | | | APIEKTISKAKGQPREPQVYTLPPSREEMTKNQVSLTC |
| | | | | LVKGFYPSDIAVEWESNGQPENNYKTTPPVLDSDGSF |
| | | | | FLYSKLTVDKSRWQQGNVFSCSVMHEALHNHYTQKS |
| | | | | LSLSP |
| 261. | scFc-5 | Artificial | aa | DKTHTCPPCPAPELLGGPSVFLFPPKPKDTLMISRTPE |
| | | | | VTCVVVDVSHEDPEVKFNWYVDGVEVHNAKTKPREE |
| | | | | QYGSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGKGGGGSGGGGSGGGGSGGGGSGGGGSGG |
| | | | | GGSDKTHTCPPCPAPELLGGPSVFLFPPKPKDTLMIS |
| | | | | RTPEVTCVVVDVSHEDPEVKFNWYVDGVEVHNAKTK |
| | | | | PREEQYGSTYRVVSVLTVLHQDWLNGKEYKCKVSNK |
| | | | | ALPAPIEKTISKAKGQPREPQVYTLPPSREEMTKNQVS |
| | | | | LTCLVKGFYPSDIAVEWESNGQPENNYKTTPPVLDSD |
| | | | | GSFFLYSKLTVDKSRWQQGNVFSCSVMHEALHNHYT |
| | | | | QKSLSLSPGK |
| 262. | scFc-6 | Artificial | aa | DKTHTCPPCPAPELLGGPSVFLFPPKPKDTLMISRTPE |
| | | | | VTCVVVDVSHEDPEVKFNWYVDGVEVHNAKTKPREE |
| | | | | QYGSTYRVVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGGGGSGGGGSGGGGSGGGGSGGGGSGGGG |
| | | | | SDKTHTCPPCPAPELLGGPSVFLFPPKPKDTLMISRTP |
| | | | | EVTCVWVDVSHEDPEVKFNWYVDGVEVHNAKTKPRE |
| | | | | EQYGSTYRVVSVLTVLHQDWLNGKEYKCKVSNKALP |
| | | | | APIEKTISKAKGQPREPQVYTLPPSREEMTKNQVSLTC |
| | | | | LVKGFYPSDIAVEWESNGQPENNYKTTPPVLDSDGSF |
| | | | | FLYSKLTVDKSRWQQGNVFSCSVMHEALHNHYTQKS |
| | | | | LSLSP |
| 263. | scFc-7 | Artificial | aa | DKTHTCPPCPAPELLGGPSVFLFPPKPKDTLMISRTPE |
| | | | | VTCVVVDVSHEDPEVKFNWYVDGVEVHNAKTKPCEE |
| | | | | QYNSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGKGGGGSGGGGSGGGGSGGGGSGGGGSGG |
| | | | | GGSDKTHTCPPCPAPELLGGPSVFLFPPKPKDTLMIS |
| | | | | RTPEVTCVVVDVSHEDPEVKFNWYVDGVEVHNAKTK |
| | | | | PCEEQYNSTYRCVSVLTVLHQDWLNGKEYKCKVSNK |
| | | | | ALPAPIEKTISKAKGQPREPQVYTLPPSREEMTKNQVS |
| | | | | LTCLVKGFYPSDIAVEWESNGQPENNYKTTPPVLDSD |
| | | | | GSFFLYSKLTVDKSRWQQGNVFSCSVMHEALHNHYT |
| | | | | QKSLSLSPGK |
| 264. | scFc-8 | Artificial | Aa | DKTHTCPPCPAPELLGGPSVFLFPPKPKDTLMISRTPE |
| | | | | VTCVVVDVSHEDPEVKFNWYVDGVEVHNAKTKPCEE |
| | | | | QYNSTYRCVSVLTVLHQDWLNGKEYKCKVSNKALPA |
| | | | | PIEKTISKAKGQPREPQVYTLPPSREEMTKNQVSLTCL |
| | | | | VKGFYPSDIAVEWESNGQPENNYKTTPPVLDSDGSFF |
| | | | | LYSKLTVDKSRWQQGNVFSCSVMHEALHNHYTQKSL |
| | | | | SLSPGGGGSGGGGSGGGGSGGGGSGGGGGGGG |
| | | | | SDKTHTCPPCPAPELLGGPSVFLFPPKPKDTLMISRTP |
| | | | | EVTCVWVDVSHEDPEVKFNWYVDGVEVHNAKTKPCE |
| | | | | EQYNSTYRCVSVLTVLHQDWLNGKEYKCKVSNKALP |
| | | | | APIEKTISKAKGQPREPQVYTLPPSREEMTKNQVSLTC |
| | | | | LVKGFYPSDIAVEWESNGQPENNYKTTPPVLDSDGSF |
| | | | | FLYSKLTVDKSRWQQGNVFSCSVMHEALHNHYTQKS |
| | | | | LSLSP |

What is claimed is:

1. A method of preparing a lyophilized bispecific molecule composition, the method comprising (a) inducing ice nucleation in a liquid bispecific molecule composition having a concentration of a bispecific molecule of at least about 10 mg/ml in a vial exposed to a first temperature of about −18° C. to about −10° C. for a first period of time of about 60 minutes to about 270 minutes (b) exposing the vial of step (a) to a second temperature of about −25° C. to −50° C. for a second period of time of about 1 hour to about 5 hours;
(c) drying the composition of step (b) at a third temperature of about −5° C. to about −25° C. for a third period of time of about 25 hours to about 70 hours; and
(d) further drying the composition of step (c) at a fourth temperature of about 25° C. to about 50° C. for a fourth period of time of about 4 hours to about 12 hours, resulting in a vial comprising a lyophilized bispecific molecule composition having a percental content of high molecular weight species (HMWS) of less or equal 1.5% (m/V), wherein the bispecific molecule comprises at least three domains, wherein:
the first domain binds to a tumor antigen on a target cell;
the second domain binds to an extracellular epitope of the human and/or the Macaca CD3ε chain; and
the third domain is fused to the second domain by a peptide linker, which third domain comprises two polypeptide monomers, each comprising a hinge, a CH2 and a CH3 domain, wherein said two polypeptide monomers are fused to each other via a peptide linker.

2. The method of claim 1, wherein step (a) comprises exposing the vial to the first temperature for about 90 minutes to about three hours.

3. The method of claim 2, wherein step (a) comprises exposing the vial to the first temperature for about 90 minutes to about two hours, preferably 110 minutes.

4. The method of any one of claims 1-3, wherein the first temperature in step (a) is about −12° C. to about −17° C.

5. The method of claim 4, wherein the first temperature in step (a) is about −15° C.

6. The method of any one of claims 1-5, wherein step (a) further comprises holding the ice-nucleated protein composition at the first temperature for a post-nucleation period of time of up to 90 minutes.

7. The method of claim 6, wherein the post-nucleation period of time is about 20 minutes to about 90 minutes.

8. The method of claim 7, wherein the post-nucleation period of time is about 30 minutes.

9. The method of any one of claims 1-8, where step (b) comprises exposing the vial to a second temperature of about −45° C.

10. The method of any one of claims 1-9, wherein the second period of time is about two hours to about four hours.

11. The method of claim 10, wherein the second period of time is about three hours.

12. The method of any one of claims 1-11, wherein the first temperature is transitioned to the second temperature at a rate of about 0.01° C. per minute to about 0.5° C. per minute.

13. The method of claim 12, wherein the first temperature is transitioned to the second temperature at a rate of about 0.2° C. per minute.

14. The method of any one of claims 1-13, wherein the third temperature of step (c) is about 0° C. to about −20° C.

15. The method of claim 14, wherein the third temperature of step (c) is about −5° C. to about −10° C.

16. The method of claim 15, wherein the third temperature of step (c) is about −8° C.

17. The method of any one of claims 1-9, wherein step (c) comprises exposing the vial comprising the bispecific molecule composition to an increase in temperature at rate of about 0.01° C. per minute to about 0.5° C. per minute.

18. The method of any one of claims 1-17, wherein the transition of step (b) to step (c) comprises increasing the temperature at rate of about 0.2° C. per minute to about 0.7° C. per minute and holding the vial at a temperature of about −40° C. to about −30° C. for about 15 minutes to about an hour.

19. The method of any one of claims 1-18, wherein the liquid bispecific molecule composition is exposed in step (a) to a first temperature of about −15° C. for about 110 minutes plus about 30 minutes post nucleation, is exposed in step (b) to a second temperature of about −45° C. for about 3 hours; the composition of step (b) is dried in step (c) at a third temperature of about −8° C. for about 50 hours; and the composition of (c) is further dried at a fourth temperature of about 40° C. for about 8 hours.

20. The method of any one of claims 1-18, wherein the bispecific molecule is a single chain molecule.

21. The method of any of claims 1-18, wherein the bispecific molecule is present in the composition at a concentration of at about 10 mg/ml to about 30 mg/ml or of about 20 mg/ml to about 30 mg/ml, preferably about 15 mg/ml to about 25 mg/ml.

22. The method of any one of claims 1-18, wherein the method results in a vial comprising a lyophilized bispecific molecule composition showing aggregation in terms of high molecular weight species (HMSW) formation lower than or equal to about 1.5% (m/V) or preferably lower than or equal of about 11% (m/V).

23. The method of any one of claims 1-22, wherein said third domain comprises in an amino to carboxyl order:
hinge-CH2-CH3-linker-hinge-CH2-CH3.

24. The method of any one of claims 1-23, wherein each of said polypeptide monomers of the third domain has an amino acid sequence that is at least 90% identical to a sequence selected from the group from the group consisting of: SEQ ID NO: 249-256 or identical to a sequence selected from the group from the group consisting of: SEQ ID NO: 249-256.

25. The method of any one of claims 1-24, wherein the first domain binds to CD33, CDH19, MSLN, FLT3, BCMA, CD19, MUC17, CDH3, CLDN18.2, CD70, EGFRviii, EpCAM, DLL3 and/or PSMA.

26. The method of any one of claims 1-25, wherein the first binding domain of the bispecific molecule comprises a VH region comprising CDR-H1, CDR-H2 and CDR-H3 and a VL region comprising CDR-L1, CDR-L2 and CDR-L3 selected from the group consisting of:
(a) CDR-H1 as depicted in SEQ ID NO: 4, CDR-H2 as depicted in SEQ ID NO: 5, CDR-H3 as depicted in SEQ ID NO: 6, CDR-L1 as depicted in SEQ ID NO: 1, CDR-L2 as depicted in SEQ ID NO: 2 and CDR-L3 as depicted in SEQ ID NO: 3,
(b) CDR-H1 as depicted in SEQ ID NO: 29, CDR-H2 as depicted in SEQ ID NO: 30, CDR-H3 as depicted in SEQ ID NO: 31, CDR-L1 as depicted in SEQ ID NO: 34, CDR-L2 as depicted in SEQ ID NO: 35 and CDR-L3 as depicted in SEQ ID NO: 36,
(c) CDR-H1 as depicted in SEQ ID NO: 42, CDR-H2 as depicted in SEQ ID NO: 43, CDR-H3 as depicted in SEQ ID NO: 44, CDR-L1 as depicted in SEQ ID NO: 45, CDR-L2 as depicted in SEQ ID NO: 46 and CDR-L3 as depicted in SEQ ID NO: 47,
(d) CDR-H1 as depicted in SEQ ID NO: 53, CDR-H2 as depicted in SEQ ID NO: 54, CDR-H3 as depicted in SEQ ID NO: 55, CDR-L1 as depicted in SEQ ID NO: 56, CDR-L2 as depicted in SEQ ID NO: 57 and CDR-L3 as depicted in SEQ ID NO: 58,
(e) CDR-H1 as depicted in SEQ ID NO: 65, CDR-H2 as depicted in SEQ ID NO: 66, CDR-H3 as depicted in SEQ ID NO: 67, CDR-L1 as depicted in SEQ ID NO: 68, CDR-L2 as depicted in SEQ ID NO: 69 and CDR-L3 as depicted in SEQ ID NO: 70,
(f) CDR-H1 as depicted in SEQ ID NO: 83, CDR-H2 as depicted in SEQ ID NO: 84, CDR-H3 as depicted in SEQ ID NO: 85, CDR-L1 as depicted in SEQ ID NO: 86, CDR-L2 as depicted in SEQ ID NO: 87 and CDR-L3 as depicted in SEQ ID NO: 88,
(g) CDR-H1 as depicted in SEQ ID NO: 94, CDR-H2 as depicted in SEQ ID NO: 95, CDR-H3 as depicted in SEQ ID NO: 96, CDR-L1 as depicted in SEQ ID NO: 97, CDR-L2 as depicted in SEQ ID NO: 98 and CDR-L3 as depicted in SEQ ID NO: 99,
(h) CDR-H1 as depicted in SEQ ID NO: 105, CDR-H2 as depicted in SEQ ID NO: 106, CDR-H3 as depicted in SEQ ID NO: 107, CDR-L1 as depicted in SEQ ID NO: 109, CDR-L2 as depicted in SEQ ID NO: 110 and CDR-L3 as depicted in SEQ ID NO: 111,
(i) CDR-H1 as depicted in SEQ ID NO: 115, CDR-H2 as depicted in SEQ ID NO: 116, CDR-H3 as depicted in SEQ ID NO: 117, CDR-L1 as depicted in SEQ ID NO: 118, CDR-L2 as depicted in SEQ ID NO: 119 and CDR-L3 as depicted in SEQ ID NO: 120,
(j) CDR-H1 as depicted in SEQ ID NO: 126, CDR-H2 as depicted in SEQ ID NO: 127, CDR-H3 as depicted in SEQ ID NO: 128, CDR-L1 as depicted in SEQ ID NO: 129, CDR-L2 as depicted in SEQ ID NO: 130 and CDR-L3 as depicted in SEQ ID NO: 131,
(k) CDR-H1 as depicted in SEQ ID NO: 137, CDR-H2 as depicted in SEQ ID NO: 138, CDR-H3 as depicted in SEQ ID NO: 139, CDR-L1 as depicted in SEQ ID NO: 140, CDR-L2 as depicted in SEQ ID NO: 141 and CDR-L3 as depicted in SEQ ID NO: 142,
(l) CDR-H1 as depicted in SEQ ID NO: 152, CDR-H2 as depicted in SEQ ID NO: 153, CDR-H3 as depicted in SEQ ID NO: 154, CDR-L1 as depicted in SEQ ID NO: 155, CDR-L2 as depicted in SEQ ID NO: 156 and CDR-L3 as depicted in SEQ ID NO: 157,
(m) CDR-H1 as depicted in SEQ ID NO: 167, CDR-H2 as depicted in SEQ ID NO: 168, CDR-H3 as depicted in SEQ ID NO: 169, CDR-L1 as depicted in SEQ ID NO: 170, CDR-L2 as depicted in SEQ ID NO: 171 and CDR-L3 as depicted in SEQ ID NO: 172,
(n) CDR-H1 as depicted in SEQ ID NO: 203, CDR-H2 as depicted in SEQ ID NO: 204, CDR-H3 as depicted in SEQ ID NO: 205, CDR-L1 as depicted in SEQ ID NO: 206, CDR-L2 as depicted in SEQ ID NO: 207 and CDR-L3 as depicted in SEQ ID NO: 208;
(o) CDR-H1 as depicted in SEQ ID NO: 214, CDR-H2 as depicted in SEQ ID NO: 215, CDR-H3 as depicted in SEQ ID NO: 216, CDR-L1 as depicted in SEQ ID NO: 217, CDR-L2 as depicted in SEQ ID NO: 218 and CDR-L3 as depicted in SEQ ID NO: 219;
(p) CDR-H1 as depicted in SEQ ID NO: 226, CDR-H2 as depicted in SEQ ID NO: 227, CDR-H3 as depicted in SEQ ID NO: 228, CDR-L1 as depicted in SEQ ID NO: 229, CDR-L2 as depicted in SEQ ID NO: 230 and CDR-L3 as depicted in SEQ ID NO: 231; and
(q) CDR-H1 as depicted in SEQ ID NO: 238, CDR-H2 as depicted in SEQ ID NO: 239, CDR-H3 as depicted in SEQ ID NO: 240, CDR-L1 as depicted in SEQ ID NO: 241, CDR-L2 as depicted in SEQ ID NO: 242 and CDR-L3 as depicted in SEQ ID NO: 243.

27. The method of any one of claims 1-26, wherein the first domain has an amino acid sequence selected from the group consisting of SEQ ID NOs: 7, 8, 17, 27, 28, 37, 38, 39, 40, 41, 48, 49, 50, 51, 52, 59, 60, 61, 62, 63, 64, 71, 72, 73, 74, 75. 76, 77, 78, 79, 80, 81, 89, 90, 91, 92, 93, 100, 101, 102, 103, 104, 113, 114, 121, 122,123, 124, 125, 131,132, 133, 134, 135, 136, 143,144, 145, 146, 147, 148, 149, 150, 151, 158,159, 160, 161, 162, 163, 164, 165, 166, 173, 174, 175, 176, 177, 178, 179, 180, 181, 223, 235 and 246, preferably 100 to 104.

28. The method of any one of claims 1-27, wherein ice nucleation is induced via ice-fog or depressurization.

29. A lyophilized bispecific molecule composition prepared via the method of any one of claims 1-28.
